(19) United States
(12) Patent Application Publication
(10) Pub. No.: US 2015/0174209 A1
(43) Pub. Date: Jun. 25, 2015

(54) INSULIN-PRAMLINTIDE COMPOSITIONS AND METHODS FOR MAKING AND USING THEM

(71) Applicants: AMYLIN PHARMACEUTICALS. LLC, San Diego, CA (US); UVA PATENT FOUNDATION d/b/a THE UVA LICENSING AND VENTURES GROUP, Charlottesville, VA (US); GRUPPO BIOMED MOD, Padova (IT)

(72) Inventors: Elaine Chiquette, San Antonio, TX (US); Kathrin Herrmann, San Diego, CA (US); David G. Maggs, San Diego, CA (US); Orville G. Kolterman, Poway, CA (US); Jui-Chen Lin, San Diego, CA (US); Steven Shijun Ren, San Diego, CA (US); Li Jin, San Diego, CA (US); Claudio Cobelli, Padova (IT); Boris Kovatchev, Charlottesville, VA (US)

(73) Assignees: AMYLIN PHARMACEUTICALS. LLC, San Diego, CA (US); UVA PATENT FOUNDATION d/b/a THE UVA LICENSING AND VENTURES GROUP, Charlottesville, VA (US); GRUPPO BIOMED MOD, Padova, PA (IT)

(21) Appl. No.: 14/403,177

(22) PCT Filed: May 24, 2013
(86) PCT No.: PCT/US2013/042745

§ 371 Date: Nov. 22, 2014

Related U.S. Application Data
(60) Provisional application No. 61/651,945, filed on May 25, 2012.
(60) Provisional application No. 61/773,737, filed on Mar. 6, 2013.

(51) Int. Cl. A61K 38/28; A61K 9/00; G06F 19/00; A61M 5/14; A61M 5/315; A61K 38/22; A61K 9/19

(52) U.S. Cl. CPC A61K 38/28; A61K 38/22; A61K 9/0019; A61K 9/19; A61M 5/1407; A61M 5/31596; G06F 19/3468

(57) ABSTRACT

In alternative embodiments, the invention provides formulations, pharmaceutical compositions, devices and other products of manufacture comprising a therapeutically effective mixture of an insulin and a pramlintide, and methods for making and using them. For example, methods and compositions of the invention are used in the treatment or amelioration of a diabetes, a dementia or Alzheimer's disease, any abnormality of blood glucose control, an inability to control blood glucose, an elevation of fasting glucose or Impaired Fasting Glucose (IFG), an abnormality of tolerance to a glucose load or Impaired Glucose Tolerance (IGT), a hyperglycemia induced by an illness, a trauma, a medication administration or a form of metabolic, psychological or physical stress, or a hyperglycemia induced by steroids (steroid-induced diabetes), a latent autoimmune diabetes in adults (LADA), a postprandial or reactive Hypoglycemia or an insulin resistance, a PolyCystic Ovary Syndrome (PCOS), a ketoacidosis, a gestational diabetes, a hyperkalemia, a cancer or cachexia, a beta blocker overdose, or a jaundice. In alternative embodiments, the invention provides insulin pumps, devices, subcutaneous insulin infusion therapy devices, continuous subcutaneous insulin infusion therapy devices, infusion therapy devices, reservoirs, ampoules, vials, syringes, cartridges, disposable pen or jet injectors, prefilled pens or syringes or cartridges, cartridge or disposable pen or jet injectors, two chambered or multi-chambered pumps, syringes, cartridges or pens or jet injectors, or an artificial pancreas, comprising a formulation having an insulin:pramlintide ratio of the invention.

### RELATED APPLICATIONS

This Patent Convention Treaty (PCT) International Application claims the benefit of priority under 35 U.S.C. §119(e) of U.S. Provisional Application Ser. No. 61/651,945, filed May 25, 2012; and U.S. Ser. No. 61/773,737, filed Mar. 6, 2013. The aforementioned applications are expressly incorporated herein by reference in its entirety and for all purposes.

### TECHNICAL FIELD

This invention generally relates to medicine and medical devices. In alternative embodiments, the invention provides formulations, pharmaceutical compositions, devices and other products of manufacture comprising therapeutically effective mixtures of insulin and pramlintide at specific ratios, and methods for making and using them. For example, methods and compositions of the invention are used in the treatment or amelioration of a diabetes, a dementia or Alzheimer's disease, any abnormality of blood glucose control, an inability to control blood glucose, an elevation of fasting glucose or Impaired Fasting Glucose (IFG), an abnormality of tolerance to a glucose load or Impaired Glucose Tolerance (IGT), a hyperglycemia induced by an illness, a trauma, a medication administration or a form of metabolic, psychological or physical stress, or a hyperglycemia induced by steroids (steroid-induced diabetes), latent autoimmune diabetes in adults (LADA), a postprandial or reactive hypoglycemia or an insulin resistance, a PolyCystic Ovary Syndrome (PCOS), a ketoacidosis, a gestational diabetes, a hyperkalemia, a cancer or cachexia, a beta blocker overdose, or a jaundice. In alternative embodiments, the invention provides insulin pumps, devices, subcutaneous insulin infusion therapy devices, continuous subcutaneous insulin infusion therapy devices, infusion therapy devices, reservoirs, ampoules, vials, syringes, cartridges, disposable pen or jet injectors, prefilled pens or syringes or cartridges, cartridge or disposable pen or jet injectors, two chambered or multi-chambered pumps, syringes, cartridges or pens or jet injectors comprising an insulin:pramlintide formulation of the invention.

### BACKGROUND

Pramlintide is an analogue of amylin, a small peptide hormone that is released into the bloodstream by the β-cells of the pancreas along with insulin, after a meal. Like insulin, amylin is deficient in individuals with diabetes. By augmenting endogenous amylin, pramlintide aids in the absorption of glucose by slowing gastric emptying, promoting satiety via hypothalamic receptors, and inhibiting inappropriate secretion of glucagon, a catabolic hormone that opposes the effects of insulin and amylin.

Pramlintide has been approved by the FDA, for use by patients with Type 1 and Type 2 diabetes who use insulin. Pramlintide allows patients to use less insulin, lowers average blood sugar levels, and substantially reduces what otherwise would be a large unhealthy rise in blood sugar that occurs in diabetics right after eating.

SYMLIN® is an injectable composition of the acetate salt form of pramlintide, that is formulated as a clear, isotonic, sterile solution for subcutaneous (SC) administration, with the pramlintide acetate in. Pramlintide acetate in SYMLIN® is present at either 1000 mcg/L (ug/mL) or 600 mcg/mL; a disposable multidose SYMLINPEN® pen-injector contains 1000 mcg/mL of pramlintide (as acetate); and SYMLIN® vials contain 600 mcg/mL of pramlintide (as acetate). Both formulations comprise: 2.25 mg/mL of metacresol as a preservative, D-mannitol as a tonicity modifier (at 4.3% (wt/vol), and acetic acid and sodium acetate as pH modifiers (30 mM acetate at pH of approximately 4.0).

The pharmacokinetics, pharmacodynamics, and safety of pramlintide and various insulin formulations in patients with type 1 diabetes mellitus (DM) when given as separate injections or mixed in the same syringe before injection has been studied. In two randomized, open-label studies, patients with type 1 DM received preprandial injections of pramlintide, short-acting insulin, and long-acting insulin administered either by separate injections or after mixing in various combinations. Serum free insulin and plasma glucose concentrations were measured for 10 hours and plasma pramlintide concentrations for 5 hours after injection. It was reported that mixing pramlintide with short- or long-acting insulin in the same syringe before subcutaneous injection did not affect the pharmacodynamics of glucose or the pharmacokinetics of insulin or pramlintide in a clinically significant manner.

Typical patient instructions warn not to mix pramlintide and insulin, indicating that they are not compatible, and they must be given as separate injections; or, to never mix pramlintide and insulin, and that one must use different syringes for pramlintide and insulin because insulin can affect pramlintide when the two are mixed together, see, e.g., SYMLIN Prescribing Information, PrescriptonDrugs.com, Cerner Multum, Inc. Version: 2.01. Revision date: Jun. 20, 2005, because there were some minor differences in the AUC and Cmax of Pramlintide, see e.g., Weyer et al., Am J Health-Syst Pharm, Vol 62 Apr. 15, 2005.

### SUMMARY

In alternative embodiments, the invention provides liquid pharmaceutical compositions or formulations, or reconstitutable dried pharmaceutical compositions or formulations, comprising:
(a) (i) a pramlintide or a pramlintide peptide, or a physiologically acceptable salt thereof; and
   (ii) a human insulin or a human insulin peptide (HIP) or an analog thereof, or a physiologically acceptable salt thereof,
   and optionally the human insulin, human insulin peptide (HIP), or analog thereof is or comprises: an aspart, a NOVOLOG™ or a NOVORAPID™ (Novo Nordisk, Bagsvaerd, Denmark); a glulisine or an APIDRA™ (Sanofi S.A., Paris, France); a lispro, an insulin lispro protamine or a HUMALOG™ (Eli Lilly and Company, Indianapolis, Ind.); a HUMULIN R™, a HUMULIN N™, a HUMULIN 70/30™ or a HUMULIN 70/30™ (Eli Lilly and Company, Indianapolis, Ind.);
   or a regular (wild type) isolated or a recombinant human insulin, or a fast-acting human insulin analog or variant thereof,
   and optionally the pramlintide peptide comprises or consists of a C-terminal amide form of a peptide

(SEQ ID NO: 1)
KCNTATCATQRLANFLVHSSNNFGPILPPTNVGSNTY;

wherein the ratio of the pramlintide or pramlintide peptide to the human insulin, human insulin peptide (HIP) in the liquid, reconstitutable dried pharmaceutical composition or formulation is:
4 μgm:1 U; or 5.92 mole insulin to 1 mole pramlintide;
4.5 μgm:1 U; or 5.26 mole insulin to 1 mole pramlintide;
5 μgm:1 U; or 4.74 mole insulin to 1 mole pramlintide;
5.5 μgm:1 U; or 4.31 mole insulin to 1 mole pramlintide;
6 μgm or 1.52 nmoles pramlintide: 1 U (international unit) or 6.0 nmoles human insulin or equivalent nmoles of human insulin peptide providing 1 U of insulin activity, or 0.25 mole pramlintide to 1 mole human insulin (6 μgm:1 U) or moles of human insulin peptide providing the same unit of activity as 0.25 mole human insulin, or 3.98 mole human insulin or equivalent moles of human insulin peptide to 1 mole pramlintide;
6.5 μgm:1 U; or 3.63 mole insulin to 1 mole pramlintide;
7 μgm:1 U; or 3.38 mole insulin to 1 mole pramlintide;
8 μgm:1 U; or 2.96 mole insulin to 1 mole pramlintide;
8.5 μgm:1 U; or 2.79 mole insulin to 1 mole pramlintide;
9 μgm:1 U; or 2.63 mole insulin to 1 mole pramlintide;
9.5 μgm:1 U; or 2.49 mole insulin to 1 mole pramlintide;
10 μgm:1 U; or 2.37 mole insulin to 1 mole pramlintide;
11 μgm:1 U; or 2.15 mole insulin to 1 mole pramlintide;
12 μgm:1 U; or 1.97 mole insulin to 1 mole pramlintide;
13 μgm:1 U; or 1.82 mole insulin to 1 mole pramlintide;
14 μgm:1 U; or 1.69 mole insulin to 1 mole pramlintide;
15 μgm:1 U; or 1.58 mole insulin to 1 mole pramlintide;
16 μgm:1 U; or 1.48 mole insulin to 1 mole pramlintide;
17 μgm:1 U; or 1.39 mole insulin to 1 mole pramlintide;
18 μgm:1 U; or 1.31 mole insulin to 1 mole pramlintide;
19 μgm:1 U; or 1.25 mole insulin to 1 mole pramlintide;
20 μgm:1 U; or 1.18 mole insulin to 1 mole pramlintide;
21 μgm:1 U; or 1.13 mole insulin to 1 mole pramlintide;
22 μgm:1 U; or 1.08 mole insulin to 1 mole pramlintide;
23 μgm:1 U; or 1.03 mole insulin to 1 mole pramlintide;
24 μgm:1 U, or 0.99 mole insulin to 1 mole pramlintide; or
between about 4 or 5 μgm:1 U to about 24 μgm:1 U,
between about 5.5 μgm:1 U to about 16 μgm:1 U,
between about 6 μgm:1 U to about 12 μgm:1 U,
between about 7 μgm:1 U to about 24 μgm:1 U,
between about 7.5 μgm:1 U to about 16 μgm:1 U,
between about 8 μgm:1 U to about 9, 10, 11 or 12 μgm:1 U,
and the liquid pharmaceutical composition or formulation has a pH of between about 3.3 to 4.3, about 3.0 and 5.5, about 3.5 to 4.5, about 3.7 to about 4.35, about 4.0, or a pH of about 3.0, 3.1, 3.2, 3.3, 3.4, 3.5, 3.6, 3.7, 3.8, 3.9, 4.0, 4.05, 4.1, 4.15, 4.2, 4.25, 4.3, 4.35 or 4.4,
or optionally, when the reconstitutable dried pharmaceutical composition or formulation is reconstituted, it has a pH of between about 3.3 to 4.3, about 3.0 and 5.5, about 3.5 to 4.5, about 3.7 to about 4.35, about 4.0, or a pH of about 3.0, 3.1, 3.2, 3.3, 3.4, 3.5, 3.6, 3.7, 3.8, 3.9, 4.0, 4.05, 4.1, 4.15, 4.2, 4.25, 4.3, 4.35 or 4.4;
or optionally, the pH range is more than 3.5 to less than 4.4, or 3.8 to less than 4.4; or pramlintide peptide formulations and final co-formulation pH include about 3.8, 3.85, 3.9, 3.95, 4.0, 4.05, 4.1, 4.15, 4.2, 4.25, 4.3, and 4.35; or the pH range is each range that is selected from the group of ranges where the pH values 3.8, 3.85, 3.9, 3.95, 4.0, 4.05, 4.1, 4.15, 4.2, 4.25, 4.3, and 4.35 are selected as a lower and an upper end of the range, including for example 3.8 to 4.35, 3.85 to 4.35, 3.85 to 4.2, 3.85 to 4.15, 3.8 to 4.2, 3.9 to 4.1, 3.9 to 4.0, and 4.0 to 4.1; or the pH range is about pH 3.9, 4.0 and 4.1, and ranges 3.9 to 4.1, 3.9 to 4.0, and 4.0 to 4.1,
wherein when calculating the ratios, a weight of the pramlintide or pramlintide peptide is based on the weight of pramlintide acetate, and an International Unit (U) of human insulin is based on U of human insulin as formulated using a HUMULIN R™ at pH 7.4;
(b) the liquid or reconstitutable dried pharmaceutical composition or formulation of (a), wherein the pramlintide or pramlintide peptide is or comprises a salt form, and optionally the pramlintide or pramlintide peptide is an acetate salt, or a trifluoroacetate (TFA) salt, or a chloride salt, or a mixture thereof;
(c) the liquid or reconstitutable dried pharmaceutical composition or formulation of (a) or (b), wherein the human insulin or human insulin peptide (HIP) is complexed with a metal ion;
(d) the liquid or reconstitutable dried pharmaceutical composition or formulation of (c), wherein the human insulin or human insulin peptide (HIP) is complexed with a zinc or a Zn⁺²;
(e) the liquid or reconstitutable dried pharmaceutical composition or formulation of (d), wherein the human insulin or human insulin peptide (HIP) is complexed with the zinc in a ratio of molar ratio of at least 6:2;
(f) the liquid or reconstitutable dried pharmaceutical composition or formulation of (d) or (e), wherein the human insulin or human insulin peptide (HIP) is complexed with the zinc and is substantially hexameric;
(g) the liquid or reconstitutable dried pharmaceutical composition or formulation of (f), wherein the human insulin or human insulin peptide (HIP) is complexed with the zinc and the insulin is greater than about 95%, 96%, 97%, 98%, 99% or more hexameric, or is between about 90% and 100% hexameric;
(h) the liquid or reconstitutable dried pharmaceutical composition or formulation of any of (a) to (g), wherein the pramlintide or pramlintide peptide or the insulin or human insulin peptide (HIP) is a recombinant peptide,
and optionally the recombinant peptide is produced in a prokaryote or a eukaryote, and optionally the prokaryote is an E. coli, and optionally the eukaryote is a yeast; and optionally the yeast is a Saccharomyces or a Pichia,
and optionally where the human insulin or HIP are comprised of an A chain and a B chain, the A chain and B chain are separately synthesized or recombinantly produced, and optionally the recombinant A chain and B chain are synthesized in the same cell;
(i) the liquid pharmaceutical composition or formulation of any of (a) to (h), comprising: a liquid vehicle comprising a water, or an aqueous or an organic solvent mixture, or an substantially isotonic aqueous or organic solvent mixture;
(j) the liquid or reconstitutable dried pharmaceutical composition or formulation of the invention, further comprising a pharmaceutically acceptable excipient;
(k) the liquid or reconstitutable dried pharmaceutical composition or formulation of any of (a) to (i), further comprising a buffer,
and optionally the buffer comprises an acetate, a phosphate, a citrate, a tartrate, or a glutamate buffer, or a mixture or a combination thereof,
and optionally the buffer is between about 0.02 to 0.5% (w/v) of an acetate, phosphate, citrate, tromethamine or glutamate buffer, or the buffer has an acetate concentration of between about 3.4 and 84.7 mM, a phosphate concentration of between about 2.1 and 52.6 mM, a citrate concentration of between about 1.1 and 26.4 mM, or a glutamate concentration of between about 1.4 and 34.2 mM;
(l) the liquid or reconstitutable dried pharmaceutical composition or formulation of (k), wherein the buffer is an acetate buffer, and optionally the acetate is formulated at between about 15 to 20 mM, 17 to 25 mM, 25 to 65 mM, or about 25 to 80 mM or is formulated at about 15 mM, 16 mM, 17 mM, 20 mM, 25 mM, 30 mM, 40 mM, 50 mM, 60 mM, 70 mM, 80 mM, 90 mM, 100 mM, 110 mM or 120 mM,
and optionally the buffer does not or substantially does not chelate a zinc, and optionally for the reconstitutable dried pharmaceutical composition or formulation, the buffer is a non-volatile buffer;
(m) the liquid or reconstitutable dried pharmaceutical composition or formulation of any of (k) to (l), wherein the buffer is present at a concentration providing a buffer capacity equivalent to the buffer capacity of sodium acetate buffer formulated at between about 15 to 20 mM, 17 to 25 mM, 25 to 65 mM, 25 to 80 mM, or at about 15 mM, 16 mM, 17 mM, 20 mM, 25 mM, 30 mM, 40 mM, 50 mM, 60 mM, 70 mM, 80 mM, 90 mM, 100 mM, 110 mM or 120 mM,
(n) the liquid or reconstitutable dried pharmaceutical composition or formulation of any of (a) to (m), further comprising an isotonicity agent or a bulking agent, wherein optionally the isotonicity agent or bulking agent is or comprises a sodium chloride, a carbohydrate, a polyol, or a polyhydric alcohol or a combination or mixture thereof;
(o) the liquid or reconstitutable dried pharmaceutical composition or formulation of (n), wherein the isotonicity agent carbohydrate or polyhydric alcohol or amino acid is formulated as a substantially isotonic formulation, optionally at about 1.0 to 10% (w/v) of the carbohydrate or the polyhydric alcohol or amino acid;
(p) the liquid or reconstitutable dried pharmaceutical composition or formulation of any of (n) to (o), wherein the polyhydric alcohol comprises a mannitol (D-mannitol), a sorbitol, an inositol, a glycerol, a xylitol, an ethylene glycol, a propylene/ethylene glycol copolymer, a PEG 8000, a PEG 400, a PEG 4000, a PEG 200, a PEG 1450 or a PEG 3350, or a combination thereof;
(q) the liquid or reconstitutable dried pharmaceutical composition or formulation of any of (n) to (p), wherein the carbohydrate comprises a mannitol, a mannose, a ribose, a trehalose, a maltose, a glycerol, a inositol, a lactose, a sucrose, a fructose, a galactose, or an arabinose, or a mixture or a combination thereof;
(r) the liquid or reconstitutable dried pharmaceutical composition or formulation of any of (a) to (q), wherein further comprising a glycerol, a glycerin, a mannitol, glycine or a mixture or a combination thereof,
and optionally the glycerol, when present, is between about 12 to 20 mg/ml, or about 16 mg/ml, and the mannitol, when present, is between about 3% to 6%, or about 4.3% (w/v);
(s) the liquid or reconstitutable dried pharmaceutical composition or formulation of any of (a) to (r), further comprising an isotonicity agent comprising a mixture of a glycerol and a mannitol;
(t) the liquid or reconstitutable dried pharmaceutical composition or formulation of any of (a) to (s), further comprising a preservative, wherein optionally the buffer is a meta-cresol (or m-cresol, m-methylphenol, or m-methylphenylol) or a phenol,
and optionally the m-cresol is formulated at between about 2 and 4 mg/mL, or at about 3 mg/mL, 2.25 mg/mL, 2.5 mg/mL or 2.0 mg/mL, wherein optionally the m-cresol is formulated at one-half of between about 2 and 4 mg/mL, or at about 3 mg/mL, 2.25 mg/mL, 2.5 mg/mL or 2.0 mg/mL, due to dilution;
(u) the liquid or reconstitutable dried pharmaceutical composition or formulation of any of (a) to (t), further comprising a metal ion, and optionally the metal ion is or comprises: a salt of a metal ion, a zinc or a Zn⁺²,
wherein optionally the metal salt is a zinc chloride, a zinc acetate, a zinc oxide and optionally the zinc chloride is formulated at about 7 micrograms/mL (mgm/mL), and optionally the Zn⁺2 is formulated at an amount equivalent to a zinc in a zinc chloride at about 7 mcg/mL, wherein optionally the zinc is formulated at about 0.015 mg/100 units;
(v) the liquid or reconstitutable dried pharmaceutical composition or formulation of any of (a) to (u), further comprising a surfactant,
and optionally the surfactant comprises a polyoxyethylene (20) sorbitan monolaurate, a polyoxyethylene (20) sorbitan monooleate, a 3-[(3-cholamidopropyl) dimethylammonio]1-propanol sulfonate, a polyoxyethylene (23) lauryl ether, a poloxamer or a non-ionic surfactant or a mixture or combination thereof;
(w) the liquid or reconstitutable dried pharmaceutical composition or formulation of any of (a) to (v), wherein the dried pharmaceutical composition or formulation is prepared by spray drying, rotary evaporation, freeze-drying or lyophilization;
(x) the liquid or reconstitutable dried pharmaceutical composition or formulation of any of (a) to (w), comprising or formulated as: an aqueous solution, an injectable solution, an aqueous or an organic solvent mixture, a suspension, a lozenge, a capsule, a gel, a geltab, a nanosuspension, a nanoparticle, a microgel and/or a spray or an aerosol;
(y) the liquid or reconstitutable dried pharmaceutical composition or formulation of any of (a) to (x), comprising or packaged in: a continuous subcutaneous insulin infusion therapy device; an insulin pump device; an ampoule; a vial; a cartridge; a syringe, cartridge or disposable pen or jet injector; a needleless injector or a needle free injector; a prefilled pen or syringe or cartridge, or a disposable syringe or pen or jet injector; an AUTOPEN™; a two chambered syringe, cartridge or disposable pen or jet injector; a multi-chambered syringe, cartridge or disposable pen or jet injector;
(z) the liquid or reconstitutable dried pharmaceutical composition or formulation of any of (a) to (y), further comprising instructions for using the liquid pharmaceutical composition or formulation to treat a patient, wherein optionally the patient is being treated for, and the instructions are for use of the liquid or reconstitutable dried pharmaceutical composition or formulation for treating:
   a diabetes mellitus (diabetes), wherein optionally the diabetes mellitus is Type 1 diabetes or Type 2 diabetes, or a prediabetic condition (prediabetes),
   a dementia or Alzheimer's disease,
   an abnormality of blood glucose control, or inability to control blood glucose
   an elevation of fasting glucose or Impaired Fasting Glucose (IFG),
   an abnormality of tolerance to a glucose load or Impaired Glucose Tolerance (IGT),
   a hyperglycemia induced by an illness, a trauma, a medication administration or a form of metabolic, psychological or physical stress, or a hyperglycemia induced by steroids (steroid-induced diabetes),
   a latent autoimmune diabetes in adults (LADA),
   a postprandial or reactive Hypoglycemia or an insulin resistance,
   a PolyCystic Ovary Syndrome (PCOS),
   a ketoacidosis,
   a gestational diabetes,
   a hyperkalemia,
   a cancer or cachexia,
   a beta blocker overdose, or
   a jaundice,
and optionally the patient is being treated with a basal insulin, or the insulin is administered to maintain a basal insulin level,
and optionally the patient is treated with an oral or injectable anti-diabetic medicine, or one or more other medications, or the patients can be those naïve to insulin other anti-diabetes medicines, and whether naïve or not, the formulations of the invention can be the patients only anti-diabetes medication;
(aa) the liquid or reconstitutable dried pharmaceutical composition or formulation of any of (a) to (z), wherein the liquid insulin concentration is at about 100 Units/mL, 200 Units/mL, 300 Units/mL, 400 Units/mL, 500 Units/mL or 600 Units/mL, or is between about 100 Units/mL to about 600 Units/mL,
or the reconstitutable dried pharmaceutical composition or formulation is formulated such that upon reconstitution the liquid insulin concentration will be at about 100 Units/mL, 200 Units/mL, 300 Units/mL, 400 Units/mL, 500 Units/mL or 600 Units/mL, or will be between about 100 Units/mL to about 600 Units/mL,
and optionally the reconstitutable dried pharmaceutical composition or formulation is reconstituted by a health practitioner or by a pharmacist, or is reconstituted by a patient;
(bb) the liquid or reconstitutable dried pharmaceutical composition or formulation of any of (a) to (aa), wherein the liquid formulation or the reconstituted pharmaceutical composition or formulation is usable by a patient for 1 day to 1 month, or for 1 day to 7 days, or for 1 day to 3 days, or for 1 day, 3 days, 1 week, 2 weeks or 1 month; or
(cc) the liquid or reconstitutable dried pharmaceutical composition or formulation of any of (a) to (bb), comprising or consisting of:
a formulation as set forth in FIGS. 8 to 11, 34A, 34B, 34C, 34D or 34E, or FIG. 35; or
an insulin 100 U/mL, pramlintide 600 microgram/mL, in 30 mM acetate buffer pH 4, 0.225% metra-cresol, 4.3% mannitol; or
   an insulin 100 U/mL, pramlintide 900 microgram/mL, in 30 mM acetate buffer pH 4, 0.225% metra-cresol, 4.3% mannitol; or
   a lyophilized insulin 1000 U powder with bulking agent+10 mL of Pramlintide solution where Pramlintide 600 microgram/mL in 30 mM acetate buffer pH 4, 0.225% metra-cresol, 4.3% mannitol; or
   a lyophilized insulin 1000 U powder with bulking agent+10 mL of Pramlintide solution where Pramlintide 900 microgram/mL in 30 mM acetate buffer pH 4, 0.225% metra-cresol, 4.3% mannitol.

In alternative embodiments, the invention provides devices or products of manufacture, subcutaneous insulin infusion therapy devices; continuous subcutaneous insulin infusion therapy device; an insulin pump device; an ampoule; a vial; a cartridge; a syringe, cartridge or disposable pen or jet injector; a needleless injector or a needle free injector; a prefilled syringe or pen or cartridge, or a disposable pen or syringe or jet injector; a two chambered syringe, cartridge or disposable pen or jet injector; a multi-chambered syringe, cartridge or disposable pen or jet injector; or a kit, comprising:
(a) the liquid or reconstitutable dried pharmaceutical composition or formulation of the invention,
wherein optionally the human insulin or human insulin peptide (HIP) and the pramlintide or pramlintide peptide are co-formulated together as a liquid, or
optionally the human insulin or human insulin peptide (HIP) and the pramlintide or pramlintide peptide are co-formulated together as a reconstitutable dried pharmaceutical composition or formulation, or
optionally the human insulin or human insulin peptide (HIP) and the pramlintide or pramlintide peptide are separately formulated and are stored or self-contained separately before mixing to comprise a liquid pharmaceutical composition or formulation of the invention;
(b) the device or product of manufacture of (a) or (b), further comprising an actuator, a valve, a shunt, a directional channel or equivalent thereof, or an apparatus capable of delivering or administrating to a patient or an individual an therapeutically effective dosage equivalent to a dosage of the liquid or reconstitutable dried pharmaceutical composition or formulation of the invention;
(c) the device or product of manufacture of (a) or (b), wherein the human insulin or human insulin peptide (HIP) and the pramlintide or pramlintide peptide are separately formulated and are stored separately,
and optionally the human insulin or human insulin peptide (HIP) and the pramlintide or pramlintide peptide are stored separately in separate, different or multicompartment ampoules, capsules, compartments, vials, sections, cartridges, or equivalents thereof, or in separate sections or areas of a multi-compartment cartridge, ampoule, vial or capsule or equivalents thereof,
and optionally the device or product of manufacture can deliver, or is configured to deliver, the pramlintide or pramlintide peptide and the human insulin or human insulin peptide (HIP) at a ratio as set forth in the invention,
and optionally the actuator, a valve, a shunt, a directional channel or equivalent thereof are manufactured or configured to deliver, the pramlintide or pramlintide peptide and the human insulin or human insulin peptide (HIP) at a ratio as set forth in the invention,
and optionally the actuator, a valve, a shunt, a directional channel or equivalent thereof are operably linked to a computer system, a non-transitory memory medium, a computer-readable storage medium or a computer program storage device, or an equivalent thereof, to deliver the pramlintide or pramlintide peptide and the human insulin or human insulin peptide (HIP) at a ratio as set forth in the invention;
and optionally computer system, non-transitory memory medium, computer-readable storage medium or computer program storage device, or equivalent thereof are operably linked to or are built into or are part of the device or product of manufacture;
(d) the device or product of manufacture of any of (a) to (c), wherein:
the pramlintide or pramlintide peptide is in a liquid formulation and the human insulin or human insulin peptide (HIP) is formulated in a dried formulation, such that when the pramlintide or pramlintide peptide and insulin or insulin peptide are mixed the mixture comprises a liquid formulation as set forth in the invention,
the human insulin or human insulin peptide (HIP) is in a liquid formulation and the pramlintide or pramlintide peptide is formulated in a dried formulation, such that when the pramlintide or pramlintide peptide and human insulin or human insulin peptide (HIP) are mixed the mixture comprises a liquid formulation as set forth in the invention, or
the insulin or insulin peptide and the pramlintide or pramlintide peptide are both formulated in a dried formulation, such that when the pramlintide or pramlintide peptide and human insulin or human insulin peptide (HIP) are mixed the mixture comprises a liquid formulation as set forth in the invention,
and optionally the liquid for reconstituting the dried formulation or formulations are contained in or stored in or within the device or product of manufacture, or, the device or product of manufacture is configured or manufactured to receive input of a liquid to reconstitute the dried formulation;
(e) the device or product of manufacture of any of (a) to (d), wherein the human insulin or human insulin peptide (HIP) in formulated as a liquid formulation and the insulin is a HUMULIN R™ or a NOVOLIN R™ formulation and the HIP is a HUMALOG™, NOVALOG™, or APIDRA™ formulation;
(f) the device or product of manufacture of any of (a) to (e), wherein the pramlintide or pramlintide peptide is formulated as a liquid formulation having a buffer capacity equivalent to that of at least 30 mM sodium acetate, or that of at least greater than 30 mM acetate, or at least greater than 30 mM to 80 mM sodium acetate;
(g) the device or product of manufacture of any of (a) to (f), wherein the pramlintide or pramlintide peptide is formulated in a liquid SYMLIN™ formulation;
(h) the device or product of manufacture of any of (a) to (g), wherein the pramlintide or pramlintide peptide is formulated in a liquid SYMLIN™ formulation that optionally further comprises a buffer capacity equivalent to that of at least 30 mM sodium acetate, or that of at least greater than 30 mM acetate, or at least greater than 30 mM to 80 mM sodium acetate; or
optionally the buffer comprises an acetate, a phosphate, a citrate, a tartrate, or a glutamate buffer, or a mixture or a combination thereof,
and optionally the buffer is between about 0.02 to 0.5% (w/v) of an acetate, phosphate, citrate or glutamate buffer, or the buffer has an acetate concentration of between about 3.4 and 84.7 mM, a phosphate concentration of between about 2.1 and 52.6 mM, a citrate concentration of between about 1.1 and 26.4 mM, or a glutamate concentration of between about 1.4 and 34.2 mM;
and optionally the buffer is an acetate buffer, and optionally the acetate is formulated at between about 15 to 20 mM, 17 to 25 mM, 25 to 65 mM, or about 25 to 80 mM or is formulated at about 15 mM, 16 mM, 17 mM, 20 mM, 25 mM, 30 mM, 40 mM, 50 mM, 60 mM, 70 mM, 80 mM, 90 mM, 100 mM, 110 mM or 120 mM, and optionally the buffer is present at a concentration providing a buffer capacity equivalent to the buffer capacity of sodium acetate buffer formulated at between about 15 to 20 mM, 17 to 25 mM, 25 to 65 mM, 25 to 80 mM, or at about 15 mM, 16 mM, 17 mM, 20 mM, 25 mM, 30 mM, 40 mM, 50 mM, 60 mM, 70 mM, 80 mM, 90 mM, 100 mM, 110 mM or 120 mM,
and optionally the buffer does not or substantially does not chelate a zinc, and optionally for the reconstitutable dried pharmaceutical composition or formulation, the buffer is a non-volatile buffer;
(i) the device of product wherein the liquid or reconstitutable formulation of the invention is delivered as a bolus prior to a meal to reduce the meal-associated blood glucose rise, and optionally the human insulin or HIP or the liquid or reconstitutable formulation of the invention is delivered in sufficient amounts at sufficient time intervals to maintain a basal level of insulin activity, and optionally wherein the basal level is different during waking versus sleeping.

In alternative embodiments, the invention provides methods for treating or ameliorating:
a diabetes mellitus (diabetes), wherein optionally the diabetes mellitus is Type 1 diabetes or Type 2 diabetes, or a prediabetic condition (prediabetes);
a dementia or Alzheimer's disease;
an abnormality of blood glucose control, or inability to control blood glucose,
an elevation of fasting glucose or Impaired Fasting Glucose (IFG),
an abnormality of tolerance to a glucose load or Impaired Glucose Tolerance (IGT),
a hyperglycemia induced by an illness, a trauma, a medication administration or a form of metabolic, psychological or physical stress, or a hyperglycemia induced by steroids (steroid-induced diabetes),
a latent autoimmune diabetes in adults (LADA),
a postprandial or reactive Hypoglycemia or an insulin resistance,
a PolyCystic Ovary Syndrome (PCOS),
a ketoacidosis,
a gestational diabetes,
a hyperkalemia,
a cancer or cachexia,
a beta blocker overdose, or
a jaundice,
in an individual or a patient in need of such treatment comprising:
administering a therapeutically effective amount of the liquid, reconstitutable dried pharmaceutical composition or formulation of the invention to the individual or patient in need of such treatment, or
delivering to the individual or patient in need of such treatment a pramlintide or pramlintide peptide and human insulin or human insulin peptide (HIP) formulation at a ratio as set forth in the invention using a device, a product of manufacture, an insulin pump; a subcutaneous insulin infusion therapy device, a continuous subcutaneous insulin infusion therapy device; an insulin pump device; an ampoule; a vial; a cartridge; a syringe, cartridge or disposable pen or jet injector; a needleless injector or a needle free injector; a prefilled syringe, cartridge or disposable pen or jet injector; a two chambered syringe, cartridge or disposable pen or jet injector; a multi-chambered syringe, cartridge or disposable pen or jet injector; of the invention,
and optionally a reconstitutable dried pharmaceutical composition or formulation is reconstituted by a health practitioner or by a pharmacist, or is reconstituted by a patient.
and optionally the diabetes is a Type 1 or a Type 2 diabetes; and optionally the patient is taking an additional basal insulin supplement,
and optionally the patient is treated with one or more oral or injectable anti-diabetic medicine, or one or more other medications.

In alternative embodiments, the invention provides methods for treating or ameliorating:
a diabetes mellitus (diabetes), wherein optionally the diabetes mellitus is Type 1 diabetes or Type 2 diabetes, or a prediabetic condition (prediabetes);
a dementia or Alzheimer's disease;
an abnormality of blood glucose control, or inability to control blood glucose,
an elevation of fasting glucose or Impaired Fasting Glucose (IFG),
an abnormality of tolerance to a glucose load or Impaired Glucose Tolerance (IGT),
a hyperglycemia induced by an illness, a trauma, a medication administration or a form of metabolic, psychological or physical stress, or a hyperglycemia induced by steroids (steroid-induced diabetes),
a latent autoimmune diabetes in adults (LADA),
a postprandial or reactive Hypoglycemia or an insulin resistance,
a PolyCystic Ovary Syndrome (PCOS),
a ketoacidosis,
a gestational diabetes,
a hyperkalemia,
a cancer or cachexia,
a beta blocker overdose, or
a jaundice,
in an individual or a patient in need of such treatment comprising administering a therapeutically effective amount of:
(a) (i) a pramlintide or pramlintide peptide, or a physiologically acceptable salt thereof; and
   (ii) a human insulin or a human insulin peptide (HIP) or an analog thereof, or a physiologically acceptable salt thereof,
wherein the ratio of the pramlintide or pramlintide peptide to the human insulin or human insulin peptide (HIP) is administered to the individual or patient is:
   4 μgm:1 U; or 5.92 mole insulin to 1 mole pramlintide;
   4.5 μgm:1 U; or 5.26 mole insulin to 1 mole pramlintide;
   5 μgm:1 U; or 4.74 mole insulin to 1 mole pramlintide;
   5.5 μgm:1 U; or 4.31 mole insulin to 1 mole pramlintide;
   6 μgm or 1.52 nmoles pramlintide: 1 U (international unit) or 6.0 nmoles human insulin or equivalent nmoles of human insulin peptide providing 1 U of insulin activity, or 0.25 mole pramlintide to 1 mole human insulin (6 μgm:1 U) or moles of human insulin peptide providing the same unit of activity as 0.25 mole human insulin, or 3.98 mole human insulin or equivalent moles of human insulin peptide to 1 mole pramlintide;
   6.5 μgm:1 U; or 3.63 mole insulin to 1 mole pramlintide;
   7 μgm:1 U; or 3.38 mole insulin to 1 mole pramlintide;
   8 μgm:1 U; or 2.96 mole insulin to 1 mole pramlintide;
   8.5 μgm:1 U; or 2.79 mole insulin to 1 mole pramlintide;
   9 μgm:1 U; or 2.63 mole insulin to 1 mole pramlintide;
   9.5 μgm:1 U; or 2.49 mole insulin to 1 mole pramlintide;
   10 μgm:1 U; or 2.37 mole insulin to 1 mole pramlintide;
   11 μgm:1 U; or 2.15 mole insulin to 1 mole pramlintide;
   12 μgm:1 U; or 1.97 mole insulin to 1 mole pramlintide;
   13 μgm:1 U; or 1.82 mole insulin to 1 mole pramlintide;
   14 μgm:1 U; or 1.69 mole insulin to 1 mole pramlintide;
   15 μgm:1 U; or 1.58 mole insulin to 1 mole pramlintide;
   16 μgm:1 U; or 1.48 mole insulin to 1 mole pramlintide;
   17 μgm:1 U; or 1.39 mole insulin to 1 mole pramlintide;
   18 μgm:1 U; or 1.31 mole insulin to 1 mole pramlintide;
   19 μgm:1 U; or 1.25 mole insulin to 1 mole pramlintide;
   20 μgm:1 U; or 1.18 mole insulin to 1 mole pramlintide;
   21 μgm:1 U; or 1.13 mole insulin to 1 mole pramlintide;
   22 μgm:1 U; or 1.08 mole insulin to 1 mole pramlintide;
   23 μgm:1 U; or 1.03 mole insulin to 1 mole pramlintide;
   24 μgm:1 U, or 0.99 mole insulin to 1 mole pramlintide; or
   between about 4 μgm:1 U to about 24 μgm:1 U,
   between about 5 μgm:1 U to about 24 μgm:1 U,
   between about 5.5 μgm:1 U to about 16 μgm:1 U,
   between about 6 μgm:1 U to about 12 μgm:1 U,
   between about 7 μgm:1 U to about 24 μgm:1 U,
   between about 7.5 μgm:1 U to about 16 μgm:1 U, or
   between about 8 μgm:1 U to about 9, 10, 11 or 12 μgm:1 U,
   wherein when calculating the ratios, a weight of the pramlintide or pramlintide peptide is based on the weight of pramlintide acetate, and an International Unit (U) of human insulin is based on U of human insulin as formulated using a HUMULIN R™ at pH 7.4;
(b) the method of (a), wherein the pramlintide or pramlintide peptide is or comprises a SYMLIN™;
(c) the method of (a) or (b), wherein the human insulin or human insulin peptide (HIP) is or comprises a recombinant peptide, a NOVOLIN R™, or a HUMULIN® R U-100™;
(d) the method of any of (a) to (c), wherein the human insulin or human insulin peptide (HIP) is admixed with the pramlintide or pramlintide peptide prior to administration, or simultaneously at delivery (administration) to the individual or patient, or, the admixing step is simultaneous, or concerted and sequential with administration; or
(e) the method of any of (a) to (c), wherein a reconstitutable dried pharmaceutical composition or formulation is reconstituted by a health practitioner or by a pharmacist, or is reconstituted by a patient.

In alternative embodiments, before admixing the human insulin or human insulin peptide (HIP) and the pramlintide or pramlintide peptide are stored separately, or stored separately in separate or different: insulin pumps; devices, subcutaneous insulin infusion therapy devices, continuous subcutaneous insulin infusion therapy devices, infusion therapy devices, reservoirs, ampoules, vials, syringes, cartridges, disposable pen or jet injectors, needleless injectors, needle free injectors, prefilled pens or syringes or cartridges, cartridge or disposable pen or jet injectors; or are stored in separate reservoirs or chambers in a subcutaneous insulin infusion therapy device, a continuous subcutaneous insulin infusion therapy device, a two chambered or multi-chambered pump, syringe, cartridge or pen or jet injector,
wherein optionally the separate or different insulin pumps; devices, subcutaneous insulin infusion therapy devices, continuous subcutaneous insulin infusion therapy devices, infusion therapy devices, reservoirs, ampoules, vials, cartridges, syringes, cartridges, disposable pen or jet injectors, prefilled pens or syringes or cartridges, or disposable pen or jet injectors, or needleless injectors or needle free injectors, comprise separate or at least two or more pramlintide or pramlintide peptides and insulin formulations and deliver a final pramlintide or pramlintide peptide and human insulin or human insulin peptide (HIP) formulation dosage, or a pramlintide and human insulin or human insulin peptide (HIP) effective dosage, at a P:I ratio of the invention;
an optionally the pramlintide or pramlintide peptide and human insulin or human insulin peptide (HIP) are delivered or administered a pre-meal bolus, and optionally where the human insulin or HIP or the pramlintide or pramlintide peptide and human insulin or human insulin peptide (HIP) are delivered or administered to provide a basal level of insulin activity.

In alternative embodiments, the insulin and the pramlintide or pramlintide peptide are delivered to the patient or individual using a device or product of manufacture of the invention.

In alternative embodiments, the invention provides liquids or reconstitutable dried pharmaceutical compositions or formulations for use in treating or ameliorating:
a diabetes mellitus (diabetes), wherein optionally the diabetes mellitus is Type 1 diabetes or Type 2 diabetes, or a prediabetic condition (prediabetes);
a dementia or Alzheimer's disease;
an abnormality of blood glucose control, or inability to control blood glucose,
an elevation of fasting glucose or Impaired Fasting Glucose (IFG),
an abnormality of tolerance to a glucose load or Impaired Glucose Tolerance (IGT),
a hyperglycemia induced by an illness, a trauma, a medication administration or a form of metabolic, psychological or physical stress, or a hyperglycemia induced by steroids (steroid-induced diabetes),
a latent autoimmune diabetes in adults (LADA),
a postprandial or reactive Hypoglycemia or an insulin resistance,
a PolyCystic Ovary Syndrome (PCOS),
a ketoacidosis,
a gestational diabetes,
a hyperkalemia,
a cancer or cachexia,
a beta blocker overdose, or
a jaundice,
comprising the liquid or reconstitutable dried pharmaceutical composition or formulation of the invention.

In alternative embodiments, the invention provides uses of a liquid, reconstitutable dried or lyophilized pharmaceutical composition or formulation of the invention, in the manufacture of a medicament.

In alternative embodiments, the invention provides uses of a liquid, reconstitutable dried or lyophilized pharmaceutical composition or formulation the invention, in the manufacture of a medicament for ameliorating, diminishing, treating, blocking or preventing:
a diabetes mellitus (diabetes), wherein optionally the diabetes mellitus is Type 1 diabetes or Type 2 diabetes, or a prediabetic condition (prediabetes);
a dementia or Alzheimer's disease;
an abnormality of blood glucose control, or inability to control blood glucose,
an elevation of fasting glucose or Impaired Fasting or Impaired Glucose Tolerance (IGT),
a hyperglycemia induced by an illness, a trauma, a medication administration or a form of metabolic, psychological or physical stress, or a hyperglycemia induced by steroids (steroid-induced diabetes),
a latent autoimmune diabetes in adults (LADA),
a postprandial or reactive Hypoglycemia or an insulin resistance,
a PolyCystic Ovary Syndrome (PCOS),
a ketoacidosis,
a gestational diabetes,
a hyperkalemia,
a cancer or cachexia,
a beta blocker overdose, or
a jaundice.

In alternative embodiments, the invention provides uses of the device or product of manufacture, subcutaneous insulin infusion therapy device; continuous subcutaneous insulin infusion therapy device; insulin pump device; ampoule; vial; cartridge; syringe, cartridge or disposable pen or jet injector; a needleless injector or a needle free injector; prefilled pens or syringes or cartridges, or disposable pen or jet injector; two chambered syringe, cartridge or disposable pen or jet injector; multi-chambered syringe, cartridge or disposable pen or jet injector; of the invention, in the manufacture of a medicament.

In alternative embodiments, the invention provides uses of the device or product of manufacture, subcutaneous insulin infusion therapy device; continuous subcutaneous insulin infusion therapy device; insulin pump device; ampoule; vial; cartridge; syringe, cartridge or disposable pen or jet injector; a needleless injector or a needle free injector; prefilled pens or syringes or cartridges, or disposable pen or jet injector; two chambered syringe, cartridge or disposable pen or jet injector; multi-chambered syringe, cartridge or disposable pen or jet injector; of the invention, in the manufacture of a medicament for ameliorating, diminishing, treating, blocking or preventing:
a diabetes mellitus (diabetes), wherein optionally the diabetes mellitus is Type 1 diabetes or Type 2 diabetes, or a prediabetic condition (prediabetes);
a dementia or Alzheimer's disease;
an abnormality of blood glucose control, or inability to control blood glucose,
an elevation of fasting glucose or Impaired Fasting Glucose (IFG),
an abnormality of tolerance to a glucose load or Impaired Glucose Tolerance (IGT),
a hyperglycemia induced by an illness, a trauma, a medication administration or a form of metabolic, psychological or physical stress, or a hyperglycemia induced by steroids (steroid-induced diabetes),
a latent autoimmune diabetes in adults (LADA),
a postprandial or reactive Hypoglycemia or an insulin resistance,
a PolyCystic Ovary Syndrome (PCOS),
a ketoacidosis,
a gestational diabetes,
a hyperkalemia,
a cancer or cachexia,
a beta blocker overdose, or
a jaundice.

In alternative embodiments, the invention provides therapeutic combinations of drugs comprising or consisting of a combination of at least two compounds: wherein the at least two compounds comprise or consist of:
(a) (i) a pramlintide or pramlintide peptide or a physiologically acceptable salt thereof; and
   (ii) a human insulin, or a Human Insulin Peptide (HIP), or an analog thereof, or a physiologically acceptable salt thereof,
and optionally the human insulin peptide (HIP) or analog thereof is or comprises: an aspart, a NOVOLOG™ or a NOVORAPID™ (Novo Nordisk, Bagsvaerd, Denmark); a glulisine or an APIDRA™ (Sanofi S.A., Paris, France); a lispro, an insulin lispro protamine or a HUMALOG™ (Eli Lilly and Company, Indianapolis, Ind.); a HUMULIN R™, a HUMULIN N™, a HUMULIN 70/30™ or a HUMULIN 70/30™ (Eli Lilly and Company, Indianapolis, Ind.);
or a regular (wild type) isolated or a recombinant human insulin, or a fast-acting human insulin analog or variant thereof,
and optionally the pramlintide peptide comprises or consists of a C-terminal amide form of KCNTATCATQRLANFLVHSSNNFGPILPPTNVGSNTY (SEQ ID NO:1);
wherein the ratio of the pramlintide or pramlintide peptide to the insulin or insulin peptide administered to an individual or patient is a P:I ratio of the invention;
(b) the therapeutic combination of drugs of (a), wherein the pramlintide or pramlintide peptide is or comprises pramlintide acetate; or
(c) the therapeutic combination of drugs of (a) or (b), wherein the insulin or the pramlintide or pramlintide peptide is or comprises a recombinant peptide.

In alternative embodiments, the invention provides combinations for ameliorating, diminishing, treating, blocking or preventing:
a diabetes mellitus (diabetes), wherein optionally the diabetes mellitus is Type 1 diabetes or Type 2 diabetes, or a prediabetic condition (prediabetes);
a dementia or Alzheimer's disease;
an abnormality of blood glucose control, or inability to control blood glucose,
an elevation of fasting glucose or Impaired Fasting Glucose (IFG),
an abnormality of tolerance to a glucose load or Impaired Glucose Tolerance (IGT),
a hyperglycemia induced by an illness, a trauma, a medication administration or a form of metabolic, psychological or physical stress, or a hyperglycemia induced by steroids (steroid-induced diabetes),
a latent autoimmune diabetes in adults (LADA),
a postprandial or reactive Hypoglycemia or an insulin resistance,
a PolyCystic Ovary Syndrome (PCOS),
a ketoacidosis,
a gestational diabetes,
a hyperkalemia,
a cancer or cachexia,
a beta blocker overdose, or
a jaundice,
comprising:
(a) (i) a pramlintide or pramlintide peptide or a physiologically acceptable salt thereof; and
(ii) a human insulin, or a Human Insulin Peptide (HIP), or an analog thereof, or a physiologically acceptable salt thereof,
   and optionally the human insulin peptide (HIP) or analog thereof is or comprises: an aspart, a NOVOLOG™ or a NOVORAPID™ (Novo Nordisk, Bagsværd, Denmark); a glulisine or an APIDRA™ (Sanofi S.A., Paris, France); a lispro, an insulin lispro protamine or a HUMALOG™ (Eli Lilly and Company, Indianapolis, Ind.); a HUMULIN R™, a HUMULIN N™, a HUMULIN 70/30™ or a HUMULIN 70/30™ (Eli Lilly and Company, Indianapolis, Ind.),
or a regular (wild type) isolated or a recombinant human insulin, or a fast-acting human insulin analog or variant thereof,
and optionally the pramlintide peptide comprises or consists of a C-terminal amide form of KCNTATCATQRLANFLVHSSNNFGPILPPTNVGSNTY (SEQ ID NO:1);
wherein the ratio of the pramlintide or pramlintide peptide to the insulin or insulin peptide administered to an individual or patient is a P:I ratio of the invention;
(b) the combination of (a), wherein the pramlintide or pramlintide peptide is or comprises a pramlintide acetate; or
(c) the combination of (a) or (b), wherein the insulin or the pramlintide or pramlintide peptide is or comprises a recombinant peptide.

In alternative embodiments, the invention provides computer-implemented methods capable of calculating a ratio of the amount of:
(a) a pramlintide or pramlintide peptide or a physiologically acceptable salt thereof; and
(b) a human insulin, or a Human Insulin Peptide (HIP), or an analog thereof, or a physiologically acceptable salt thereof,
to be delivered to a patient or an individual in need thereof, wherein the ratio of the pramlintide or pramlintide peptide to the insulin or insulin peptide administered to the individual or patient is a P:I ratio of the invention.

In alternative embodiments, the invention provides computer-implemented methods of processing data, wherein the method calculates a ratio of the amount of:
(a) a pramlintide or pramlintide peptide or a physiologically acceptable salt thereof; and
(b) a human insulin, or a Human Insulin Peptide (HIP), or an analog thereof, or a physiologically acceptable salt thereof,
to be delivered to a patient or individual in need thereof, comprising:
receiving data comprising the insulin level, or basal insulin level, in the patient or individual;
storing the data elements in a memory; and
calculating the ratio of the pramlintide or pramlintide peptide to the insulin or insulin peptide to be administered to the individual or patient, wherein the ratio is a P:I ratio of the invention,
and optionally the patient or individual in need thereof is under therapeutic or preventative treatment for:
a diabetes mellitus (diabetes), wherein optionally the diabetes mellitus is Type 1 diabetes or Type 2 diabetes, or a prediabetic condition (prediabetes);
a dementia or Alzheimer's disease;
an abnormality of blood glucose control, or inability to control blood glucose,
an elevation of fasting glucose or Impaired Fasting Glucose (IFG),
an abnormality of tolerance to a glucose load or Impaired Glucose Tolerance (IGT),
a hyperglycemia induced by an illness, a trauma, a medication administration or a form of metabolic, psychological or physical stress, or a hyperglycemia induced by steroids (steroid-induced diabetes),
a latent autoimmune diabetes in adults (LADA),
a postprandial or reactive Hypoglycemia or an insulin resistance,
a PolyCystic Ovary Syndrome (PCOS),
a ketoacidosis,
a gestational diabetes,
a hyperkalemia,
a cancer or cachexia,
a beta blocker overdose, or
a jaundice.

In alternative embodiments, the invention provides computer-implemented methods, further comprising being operably connected to and communicating to one or separate or different: devices, insulin pumps, subcutaneous insulin infusion therapy devices, continuous subcutaneous insulin infusion therapy devices, infusion therapy devices, reservoirs, ampoules, vials, cartridges, syringes, cartridges, disposable pen or jet injectors, prefilled pens or syringes or cartridges, or disposable pen or jet injectors, or needleless injectors or needle free injectors, wherein the pramlintide or pramlintide peptide and insulin are stored in separate reservoirs or chambers therein, and actuating or causing the insulin to be admixed with the pramlintide or pramlintide peptide prior to administration, or actuating or causing a simultaneously delivery (administration) to the individual or patient, or, actuating or causing an admixing step that is simultaneous, or concerted and sequential with administration.

In alternative embodiments, the invention provides computer program products for processing data, or a Graphical User Interface (GUI) computer program product, the computer program product comprising the method of the invention.

In alternative embodiments, the invention provides computer systems comprising a processor and a data storage device wherein said data storage device has stored thereon a computer program product for processing data, or a Graphical User Interface (GUI) computer program product, of the invention.

In alternative embodiments, the invention provides a non-transitory memory medium comprising program instructions for running, processing and/or implementing a computer program product for processing data, or a Graphical User Interface (GUI) computer program product, of the invention.

In alternative embodiments, the invention provides a computer-readable storage medium comprising a set of or a plurality of computer-readable instructions that, when executed by a processor of a computing device, cause the computing device to run, process and/or implement: a computer program product comprising the method of the invention.

In alternative embodiments, the invention provides computer program storage devices, embodied on a tangible computer readable medium, comprising: (a) a computer-implemented method of the invention; (b) a computer program product the invention; (c) a computer system of the invention; (d) a non-transitory memory medium of the invention; (e) a computer-readable storage medium of the invention; or (f) a combination thereof.

In alternative embodiments, the invention provides computers or equivalent electronic systems, comprising: a memory; and a processor operatively coupled to the memory, the processor adapted to execute program code stored in the memory to: run, process and/or implement: (a) a computer-implemented method of the invention; (b) a computer program product the invention; (c) a computer system of the invention; (d) a non-transitory memory medium of the invention; (e) a computer-readable storage medium of the invention; (f) a computer program storage device of the invention; or, (g) a combination thereof.

In alternative embodiments, the invention provides systems, comprising: a memory configured to: store values associated with a plurality of data points and/or a plurality of data elements, and a processor adapted to execute program code stored in the memory to: run, process and/or implement: (a) a computer-implemented method of the invention; (b) a computer program product the invention; (c) a computer system of the invention; (d) a non-transitory memory medium of the invention; (e) a computer-readable storage medium of the invention; (f) a computer program storage device of the invention; (g) a computer or equivalent electronic system of the invention; or, (g) a combination thereof.

In alternative embodiments, the invention provides computer-implemented systems for providing an application access to an external data source or an external server process via a connection server, and providing the ability to store values associated with the plurality of data points and/or the plurality of data elements, and an application for running, processing and/or implementing (a) a computer-implemented method of the invention; (b) a computer program product the invention; (c) a computer system of the invention; (d) a non-transitory memory medium of the invention; (e) a computer-readable storage medium of the invention; (f) a computer program storage device of the invention; (g) a computer or equivalent electronic system of the invention, or, (h) a combination thereof.

In alternative embodiments, the invention provides subcutaneous insulin infusion therapy devices; a continuous subcutaneous insulin infusion therapy device; an insulin pump device, multi-chambered syringe, cartridge or disposable pen or jet injector, comprising: (a) a computer-implemented method of the invention; (b) a computer program product the invention; (c) a computer system of the invention; (d) a non-transitory memory medium of the invention; (e) a computer-readable storage medium of the invention; (f) a computer program storage device of the invention; (g) a computer or equivalent electronic system of the invention, or, (h) a combination thereof,
and optionally comprising an actuator or apparatus capable of delivering or administrating an effective dosage to a patient or individual equivalent to a dosage of the liquid, reconstitutable dried or lyophilized pharmaceutical composition or formulation of the invention,
wherein the computer-implemented system determines or calculates and activates the delivering or administrating an effective dosage to a patient or individual equivalent,
and optionally the insulin pump device, subcutaneous insulin infusion therapy device, continuous subcutaneous insulin infusion therapy device, insulin pump, infusion therapy device, or multi-chambered syringe, cartridge or disposable pen or jet injector, or needleless injector or needle free injector, comprises separate formulations and delivers a pramlintide or pramlintide peptide and insulin formulation at a P:I ratio of the invention, as calculated and activated by: (a) a computer-implemented method of the invention; (b) a computer program product the invention; (c) a computer system of the invention; (d) a non-transitory memory medium of the invention; (e) a computer-readable storage medium of the invention; (f) a computer program storage device of the invention; (g) a computer or equivalent electronic system of the invention, or, (h) a combination thereof.

The details of one or more embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### BRIEF DESCRIPTION OF THE DRAWINGS

The drawings set forth herein are illustrative of embodiments of the invention and are not meant to limit the scope of the invention as encompassed by the claims.

FIG. 1A illustrates exemplary devices, including reusable cartridges, disposable devices, injection devices and pens, that can be used to practice this invention; and FIG. 1B illustrates an exemplary device of the invention in the form of a dual chamber injection device, e.g., a LYO-JECT™ (Vetter Pharma International USA Inc., Skokie, Ill.).

FIG. 2 illustrates exemplary human insulin peptides that can be used to practice this invention, including, e.g.: a “regular” human hormone having a 21 amino acid long “A” or alpha chain GIVEQCCTSICSLYQLENYCN (SEQ ID NO:2) and a 30 amino acid long “B” or beta chain FVNQHLCGSHLVEALYLVCGERGFFYTPKT (SEQ ID NO:3) linked by two disulfide bonds at positions; or, a lispro (e.g., can be HUMALOG® (Eli Lilly and Company, Indianapolis, Ind.)), which, as illustrated, comprises as an alpha chain SEQ ID NO:2, with the “B” or beta chain FVNQHLCGSHLVEALYLVCGERGFFYTKPT (SEQ ID NO:4); or an aspart (e.g., can be NOVOLOG® or NOVORAPID® (Novo Nordisk, Bagsværd, Denmark)), which, as illustrated, comprises as an alpha chain SEQ ID NO:2, with the “B” or beta chain FVNQHLCGSHLVEALYLVCGERGFFYTDKT (SEQ ID NO:5); or a glulisine (e.g., can be APIDRA™ (Sanofi S.A., Paris, France)), which, as illustrated, comprises as an alpha chain SEQ ID NO:2, with the “B” or beta chain FVKQHLCGSHLVEALYLVCGERGFFYTPET (SEQ ID NO:6).

FIG. 3A illustrates the pharmacokinetics of exemplary insulins that can be used to practice this invention: (a) a rapid-acting solution; (b) a short-acting solutions; (c) an intermediate-acting suspension; (d) an intermediate-acting suspension; (f) a long-acting suspension; and (e) a long-acting solution.

FIG. 3B illustrates a table summarizing the pharmacokinetics of exemplary insulins that can be used to practice this invention.

FIG. 4 illustrates alternative exemplary formulations of the invention, including their respective glycerin content, phenol content, m-cresol content, zinc content, zinc-insulin ratios, polysorbate 20 content, tromethamine content, salt content and pH.

FIG. 5 illustrates an exemplary “regular” human hormone that can be used to practice this invention having a 21 amino acid long “A” or alpha chain GIVEQCCTSICSLYQLENYCN (SEQ ID NO:2) and a 30 amino acid long “B” or beta chain FVNQHLCGSHLVEALYLVCGERGFFYTPKT (SEQ ID NO:3) linked by two disulfide bonds at positions.

FIG. 6 illustrates a table summarizing the different durations of action of exemplary insulins (including human insulin peptide (HIP) or non-HIP embodiments) that can be used to practice this invention.

FIG. 7 illustrates a table summarizing drug product profiles of an exemplary pramlintide-insulin (or human insulin peptide (HIP)) co-formulation of this invention.

FIG. 8 illustrates a table summarizing the stability results of an exemplary pramlintide-insulin co-formulation, or “mixed solution”, of this invention, noting that the physical stability of pramlintide/insulin improved with higher buffer capacity, and the pramlintide concentration was 300 mcg/ml, and insulin concentration was 100 U/ml before mixing.

FIG. 9 illustrates a table summarizing the contents of exemplary pramlintide:insulin co-formulations of the invention, using pramlintide at 300, 600 and 1200 mcg/mL (μg/mL) as “starting” or “premix” solutions, as discussed in further detail, below. In one embodiment, 0.2 ml pramlintide at 300 mcg/mL was mixed with the indicated volume of 100 U human insulin formulation of HUMULIN R™ (Eli Lilly and Company, Indianapolis, Ind.).

FIG. 10 illustrates a table summarizing the solubility and physical stability versus pH for pramlintide and human insulin, individually formulated, as discussed in further detail, below.

FIG. 11 illustrates a table summarizing alternative exemplary mixing volumes to achieve a 9:1 ratio in final formulation for a reusable cartridge or disposable device of the invention comprising pramlintide/insulin formulations; including the insulin and the pramlintide “premix” solutions (first two columns), the mixing volume in mL of the respective “premix” pramlintide and insulin formulations, and the total volume of liquid in each cartridge, as discussed in further detail, below.

FIG. 12 illustrates and summarizes an exemplary modeling effect of pramlintide using an experimental set-up from Woerle et al. (December, 2008) Diabetes Care 31(12): 2325-2331, as discussed in further detail, below.

FIG. 13 graphically illustrates the average model prediction versus (vs) Ra meal data placebo (PBO) or pramlintide (PRAM): FIG. 13A graphically illustrates the meal rate of appearance data; and FIG. 13B illustrates a table summarizing average and standard deviation (SD) values of model parameter estimates, as discussed in further detail, below.

FIG. 14A schematically illustrates a modeling scheme of pramlintide's effect on oral glucose adsorption; FIG. 14B graphically illustrates the kinetics of pramlintide's effect on oral glucose adsorption comparing pramlintide to placebo (PBO); FIG. 14C graphically illustrates the kinetics of pramlintide's effect on oral glucose adsorption by showing gastric retention comparing pramlintide to placebo (PBO); FIG. 14D graphically illustrates the kinetics of pramlintide's effect on oral glucose adsorption by comparing pramlintide to placebo (PBO) effect on glucose appearance rate due to meal ingestion (Ra meal); as discussed in further detail, below.

FIG. 15 illustrates and summarizes an exemplary method for quantifying pramlintide based on the identification of the oral glucose absorption model, where a model of the gastro-intestinal tract on Rₐ meal data of the Woerle T1D database was used (Woerle et al., December, 2008, Diabetes Care 31(12): 2325-2331), as discussed in further detail, below.

FIG. 16 graphically illustrates modeling of the pramlintide dose-response, where the dose response curve has been calculated by spanning several pramlintide-insulin ratios X on the average subject and plotting them against the percentage of glucose retained in the stomach at 120 minutes (min); the Y axis shows the percent gastric retention at 120 min post dose.

FIG. 17 graphically illustrates the results of the experiment illustrated in FIG. 26A, and FIG. 17 illustrates the average and individual glucose plasma concentration and Ra meal for the 100 virtual subjects in case pramlintide/insulin bolus ratio 9 with adjusted CR, as discussed in further detail, below.

FIG. 18 schematically illustrates a Type I diabetes simulator used to predict the effect of pramlintide on postprandial glucose in Type 1 Diabetes, as discussed in further detail, below.

FIG. 19A illustrates a table summarizing the inter-individual variability of results from the Type I diabetes simulator of FIG. 18; and FIG. 19B graphically illustrates the inter-individual variability of insulin absorption and insulin action from the Type I diabetes simulator of FIG. 18, as discussed in further detail, below.

FIG. 20 graphically illustrates generation of “virtual subjects” using the in silico model of FIG. 18 and FIG. 19, as discussed in further detail, below.

FIG. 21 and FIG. 22 illustrate and describe how to incorporate the effect of pramlintide in virtual subjects for use of the in silico model of FIG. 18 and FIG. 19, as discussed in further detail, below.

FIG. 23 illustrates and describes an exemplary in silico study design for use with the in silico model of FIG. 18 and FIG. 19, as discussed in further detail, below.

FIG. 24 graphically illustrates the result of an exemplary experiment evaluating different P-I ratios' efficacy in attenuating postprandial hyperglycemia and safety in terms of hypoglycemia, as evaluated by the Control Variability-Grid Analysis (CVGA) described below; the conclusion of this “Experiment 1” (without adjusting the subject's individual carbohydrate ratio), with data is illustrated in FIGS. 25 and 26; FIG. 24A graphically illustrates average glucose levels at different pramlintide:insulin (P:I) ratios; and FIG. 24B graphically illustrates average glucose rates of appearance at different P:I ratios, as discussed in further detail, below.

FIGS. 25A, 25B, 26A and 26B graphically illustrate data from Experiment 1 (data graphically presented in FIG. 24), where different P-I ratios' efficacy in attenuating postprandial hyperglycemia and safety in terms of hypoglycemia were evaluated by a Control Variability-Grid Analysis (CVGA), and where the in silico trial identified three optimal ratios that should result in the best glucose (BG) regulation, or maximum time spent within therapeutic range with minimum risk for hypoglycemia quantified by the Low BG Index and visualized by the Control Variability Grid Analysis; as discussed in further detail, below.

FIG. 27 graphically illustrates the result of an exemplary experiment evaluating different P-I ratios' efficacy in attenuating postprandial hyperglycemia and safety in terms of hypoglycemia, as evaluated by the Control Variability-Grid Analysis (CVGA) described below; the conclusion of this “Experiment 2”; with results graphically illustrates in FIGS. 28A, 28B, 29A and 29B, and FIGS. 41 A and 41B. In FIG. 27, P:I ratios of 3, 6, 8, 9, 10, 12 and 18 were tested adjusting the subjects' individual CR for pramlintide use to minimize hypoglycemia, FIG. 27A, the upper panel, in mg/dL; FIG. 27B, the lower panel, as mg/kg/min, as discussed in further detail, below.

FIG. 30 illustrates a table showing the average doses of pramlintide in g at the indicated P:I ratios administered with the meal of the so-called “Experiment 1” and “Experiment 2”, as discussed in further detail, below.

FIG. 31 illustrates a table showing the average percent reduction in insulin bolus needed to minimize hypoglycemia at the indicated P:I ratios, in “Experiment 1” and “Experiment 2”, as discussed in further detail, below.

FIG. 32 illustrates the Control Variability-Grid Analysis (CVGA) zone quotient used to quantify the best P-I ratio in terms of efficacy and hypoglycemia safety, as discussed in further detail, below.

FIG. 33 graphically illustrates a CVGA zone quotient plotted for placebo (no pramlintide) and P:I ratios of 3, 6, 8, 9, 10, 12 and 18; where the CVGA indicates that a P:I of 9 is optimal in terms of efficacy of post-prandial glucose control and hypoglycemia safety, as discussed in further detail, below.

FIGS. 34A and 34B illustrate a table listing exemplary final concentrations to be administered, where the concentrations of ingredients are after mixing of liquids or reconstitution of dried formulation (powder); all four exemplary formulations have a final (to be administered) pramlintide:insulin (P:I) ratio of 9:1; all four exemplary formulations have a final (to be administered) pH of 4.0; see FIG. 34F for further notes to Table 34A; and for FIG. 34F: all concentrations are the final concentrations after mixing or reconstitution unless otherwise indicated, e.g. in the “before mixing” column, as discussed in further detail, below.

FIGS. 34C and 34D illustrate a table listing the exemplary embodiment, the so-called “option 5”, that is a mix of diluent, commercially available 500 U/ml insulin and liquid commercially available SYMLIN®; option 5 can achieve final insulin concentrations less than 100 U/ml as indicated in the Figures, as discussed in further detail, below.

FIG. 34E illustrates exemplary co-formulations of the invention using varying amounts of commercially available 500 U/mL insulin and liquid pramlintide at 1000 mcg/mL and 600 mcg/mL, both of these two concentrations are commercially available in alternative commercially available SYMLIN® products, as discussed in further detail, below.

FIG. 34F provides details for the tables of FIGS. 34A, 34B, 34C, 34D and 34E, as discussed in further detail, below.

FIG. 35 illustrates a table summarizing chemical stability of exemplary pramlintide:insulin co-formulations of the invention, with the data normalized at % purity at 5° C.

FIG. 36 illustrates a table summarizing chemical stability study at 25° C. storage for 13 weeks for exemplary pramlintide:insulin co-formulations of the invention.

FIG. 37 graphically illustrates the individual calculated insulin bolus (U) in various subjects used in the Woerle protocol for quantifying pramlintide (ag)/insulin bolus (U) ratios, as discussed in further detail, below.

FIG. 38 graphically illustrates insulin bolus amounts calculated using the formula:

$$Bolus = \frac{Dose}{CR} ,$$

with CR extracted from CR distribution shown in FIG. 38 and equal to 10 g/U; the calculated bolus is then equal to 5 U, hence pramlintide/insulin bolus ratio is equal to 6; and it was concluded that pramlintide/insulin bolus ratio to be adopted in the Woerle protocol is equal to 6, as discussed in further detail, below.

FIG. 39 graphically illustrates several parameters variations distributions in the generation of virtual parameter variations for the gastro-intestinal tract model on Woerle Ra meal data, using the equations as described below; in the graphs, blue (the darker illustrated graphic) distribution are relative to parameters variations calculated from the database's estimated parameters; and the green (the lighter illustrated graphic) distribution are relative to virtual parameters variations, as discussed in further detail, below.

FIGS. 40A and 40B graphically illustrate the average and individual glucose plasma concentration and Ra meal for the 100 virtual subjects in case A, B, C and D, as discussed in further detail, below; where A is placebo, B is a P:I of 3, C is a P:I of 6, and D is a P:I of 12; FIG. 24 (Experiment 1) graphically illustrates CVGA for the 100 virtual subjects in case A, B, C and D, as discussed in further detail, below.

FIGS. 41 A and 41B, with FIG. 27, and FIGS. 28A, 28B, 29A and 29B, graphically illustrate the result of an exemplary experiment evaluating different P-I ratios' efficacy in attenuating postprandial hyperglycemia and safety in terms of hypoglycemia, as evaluated by the Control Variability-Grid Analysis (CVGA) described below; the conclusion of this “Experiment 2”, as discussed in further detail, below.

FIG. 42 graphically illustrates simulated Glucose plasma concentration and RA meal for X=[9] without CR adjustment; the data illustrates the average and individual glucose plasma concentration and Ra meal for the 100 virtual subjects in case pramlintide/insulin bolus ratio 9.

Like reference symbols in the various drawings indicate like elements, unless otherwise stated.

Reference will now be made in detail to various exemplary embodiments of the invention, examples of which are illustrated in the accompanying drawings. The following detailed description is provided to give the reader a better understanding of certain details of aspects and embodiments of the invention, and should not be interpreted as a limitation on the scope of the invention.

### DETAILED DESCRIPTION

In alternative embodiments, the invention provides formulations, pharmaceutical compositions, devices and other products of manufacture comprising or delivering an optimized therapeutically effective mixture, or ratio, of a human insulin or human insulin peptide (HIP) and a pramlintide or pramlintide peptide, and methods for making and using them.

The inventors have determined an optimized amount, or ratio, of a human insulin or human insulin peptide (HIP) and a pramlintide or pramlintide peptide (pramlintide:insulin ratio, or P:I ratio), and in alternative embodiments, the optimized P:I ratio of the invention is between about 4 mcg:1 U (μgm:1 U) and about 24 mcg:1 U, e.g., the optimized P:I amount, or P:I ratio is about 4:1, 5:1, 6:1, 7:1, 8:1, 9:1 or 10:1 to about 24:1; where previously lower or higher P:I ratios were used.

The hormone amylin is co-secreted with insulin from the pancreas in response to rising blood glucose, reportedly in approximately 1:100 amylin to insulin molar ratio, particularly during prolonged stimulation by elevated glucose. For therapeutic use, a near physiological 1:67 molar ratio of amylin to insulin has been proposed in the literature, as well as various extensive ranges of amylin to insulin ratios, including a range between 1:100 to 1:0.1 and a “most preferred” range from 1:40 to 1:1 (see for example U.S. Pat. No. 5,814,600). The inventors surprisingly found a ratio and a range of ratios of amylin agonist peptide to rapid- or regular-acting insulin different from and superior to those previously reported. Further, this invention has identified a formulation that comports with these superior ratios, allowing 1 day to multi-day use in a range of delivery devices.

In alternative embodiments, the invention provides an advantage over previously known insulin therapies by providing compositions (e.g., devices, insulin pumps, pens and the like) and methods that administer or deliver, or are capable of delivering or administering, an optimized amount, or ratio, of a human insulin or human insulin peptide (HIP) and a pramlintide or pramlintide peptide (P/I ratio), optionally as a multiple use composition, e.g., for a multi-use injection device, or a prolonged use composition, e.g., for a multi-day use pump device. In alternative embodiments, administering an optimized pramlintide or pramlintide peptide:human insulin or human insulin peptide (HIP) ratio (P/I ratio) of the invention, e.g., a ratio of a human insulin or human insulin peptide (HIP) and a pramlintide or pramlintide peptide as set forth in this invention, can reduce the amount of insulin required per gram of carbohydrate ingested per meal, or can enable a basal/bolus co-delivery, or can improve meal-associated glucose control, or can minimize undesirable or dangerous effects such as hypoglycemia, and/or improve levels of glucose; or can minimize weight gain as a potential benefit, or can improve glucose control, lessen hypoglycemia and/or weight gain, or can suppress glucagon. For example, in alternative embodiments, insulin boluses administered to a patient can be reduced by approximately 21% at a P/I ratio of about 9 μg/U (or 9 μg pramlintide or pramlintide peptide to 1 U human insulin or human insulin peptide (HIP)) to account for the in vivo effects of pramlintide and to avoid a postprandial hypoglycaemia. In alternative embodiments, the amount of insulin or insulin boluses or bolus administered to an individual can be reduced by an amount equivalent to: approximately 21% at a P/I ratio of about 9 μg/U (or 9 μg pramlintide or pramlintide peptide to 1 U human insulin or human insulin peptide (HIP)), for example, at a P/I ratio of between about 8 to 10 μg P/1 U insulin.

In alternative embodiments, the invention provides an advantage over previously known insulin therapies by providing a single formulation, e.g., a single pharmaceutical formulation, comprising an optimized amount, or ratio, of a human insulin or human insulin peptide (HIP) and a pramlintide or pramlintide peptide. The single formulation or a single pharmaceutical formulation can be a powder or a liquid. Optionally the formulation is a multiple use composition, e.g. for a multi-day injection device, or a prolonged use composition, e.g. for a multi-day use pump device.

In alternative embodiments the invention provides a single formulation, e.g., a storage-stable single formulation, of a human insulin or human insulin peptide (HIP) and a pramlintide or pramlintide peptide, and a method or composition (e.g., a device) of delivering the single formulation, at an optimized ratio of the invention, including a ratio that provides a desired activity of both human insulin or human insulin peptide (HIP) and pramlintide or pramlintide peptides. In alternative embodiments, the single formulation of the invention can reduce the number of injections required for co-therapy of insulin and pramlintide, or can reduce the amount of insulin required per gram of carbohydrate ingested per meal, or can enable a basal/bolus co-delivery, or can improve meal-associated glucose control, and/or improve basal levels of glucose. For example, in alternative embodiments, insulin or insulin boluses administered to an individual can be reduced by approximately 21% at a P/I ratio of about 9 μg/U (or 9 μg pramlintide or pramlintide peptide to 1 U human insulin or human insulin peptide (HIP)) to account for the in vivo effects of pramlintide and to avoid a postprandial hypoglycaemia.

In alternative embodiments, using a single chamber basal/bolus device of the invention to deliver an optimized ratio of the invention can increase the in-use period of the co-formulation, while minimizing undesirable or dangerous effects such as hypoglycemia. For example, in alternative embodiments, the therapeutically effective ratio of pramlintide to human insulin in a liquid or a reconstitutable dried pharmaceutical composition or formulation is 6 μgm or 1.52 nmoles pramlintide: 1 U (international unit) or 6.0 nmoles human insulin, or 0.25 mole pramlintide to 1 mole human insulin (6 μgm:1 U), or 3.95 mole human insulin to 1 mole pramlintide; or the ratio is between about 4 μgm:1 U to about 24 μgm:1 U, and intermediate ranges as provided in this invention. When human insulin is substituted by an HIP, the pramlintide to the insulin ratio expressed as weight pramlintide to units insulin activity is unchanged from that for human insulin although the molar or mass amount of the HIP per unit of insulin activity may be different than that for human insulin as would be known in the art or readily determined using known assay methods. The ratio can be provided in a co-formulation as described herein or can be provided as separate, co-administered formulations. The co-formulations enable a one-chamber device, such as a one chamber bolus/basal continuous subcutaneous injection infusion (CSII; pump) device, easing patient compliance as well as reducing device complexity and cost. In the figures and tables herein, unless otherwise noted, ratios are expressed as micrograms of pramlintide to U insulin; for example, “9:1” means 9 micrograms pramlintide to 1 U insulin activity.

### Pharmaceutical Compositions and Formulations

In alternative embodiments, the invention provides formulations and pharmaceutical compositions for practicing the methods of the invention, e.g., pharmaceutical compositions for treating or ameliorating a diabetes, a dementia, Alzheimer's disease, postprandial or reactive hypoglycemia or an insulin resistance, a PolyCystic Ovary Syndrome (PCOS), a ketoacidosis, a gestational diabetes, a hyperkalemia, a cancer or a cachexia, a beta blocker overdose, a jaundice, a cancer, septic shock, an infection, a fever, pain and related symptoms or conditions, and the like. In alternative embodiments, the invention provides compositions and methods for overcoming or diminishing or preventing a prediabetes, a gestational diabetes, a Type 1 or a Type 2 diabetes, or an abnormality of blood glucose control, or inability to control blood glucose, or an elevation of fasting glucose or Impaired Fasting Glucose (IFG), or an abnormality of tolerance to a glucose load or Impaired Glucose Tolerance (IGT), or a hyperglycemia induced by an illness, a trauma, a medication administration or a form of metabolic, psychological or physical stress, or a hyperglycemia induced by steroids (steroid-induced diabetes), latent autoimmune diabetes in adults (LADA), or a postprandial or reactive hypoglycemia or an insulin resistance.

In alternative embodiments, compositions used to practice the methods of the invention are formulated with a pharmaceutically acceptable carrier. In alternative embodiments, human insulin or human insulin peptide and pramlintide or pramlintide peptide are formulated together, or they can be formulated separately and stored separately in a device or product of manufacture of the invention; and optionally are mixed at a desired pramlintide:insulin ratio as provided for in this invention, or are injected or administered separately and substantially at the same time by a device or product of manufacture of the invention at a desired pramlintide:insulin ratio, as provided for in this invention.

The desired ratio of pramlintide:insulin can be determined manually by the subject or automatically, e.g., by use of a computer and a computer-implemented method of the invention, optionally taking into consideration basal insulin levels. In alternative embodiments, the desired ratio of pramlintide:insulin is injected or administered by bolus injection or administration of preset amounts of insulin and pramlintide in separate pens, syringes, containers, compartments and the like. In one embodiment, the pramlintide:insulin is administered intermittently an amount sufficient to maintain a basal level of insulin activity throughout a day and the pramlintide:insulin is administered at a therapeutically effective amount as a bolus prior to a meal. In one embodiment, the pramlintide:insulin is administered continuously an amount sufficient to maintain euglycemia throughout the day in a basal bolus administration.

Pramlintide Peptides and Human Insulin Peptides

In alternative embodiments, formulations of the invention comprise use of natural, synthetic, recombinant, analog or bioisostere forms of pramlintide and/or human insulin at the effective ratios of pramlintide:insulin of this invention. For example, in alternative embodiments, the final formulations of human insulin used with pramlintide or pramlintide peptide retain pharmacokinetic/pharmacodynamic (PK/PD) profiles similar to that of HUMULIN R™ and/or NOVOLIN R™ (these are formulations that contain recombinant human insulin as hexamer complexed with zinc) or for final formulations of a HIP, the PK/PD profile will be similar to that of the corresponding commercially available therapeutic HIP formulation. For example, when the HIP is lispro, the PK/PD will be similar to that of HUMALOG®; when insulin is aspart, then similar to that of NOVOLOG®; and when glulisine, similar to that of APIDRA®.

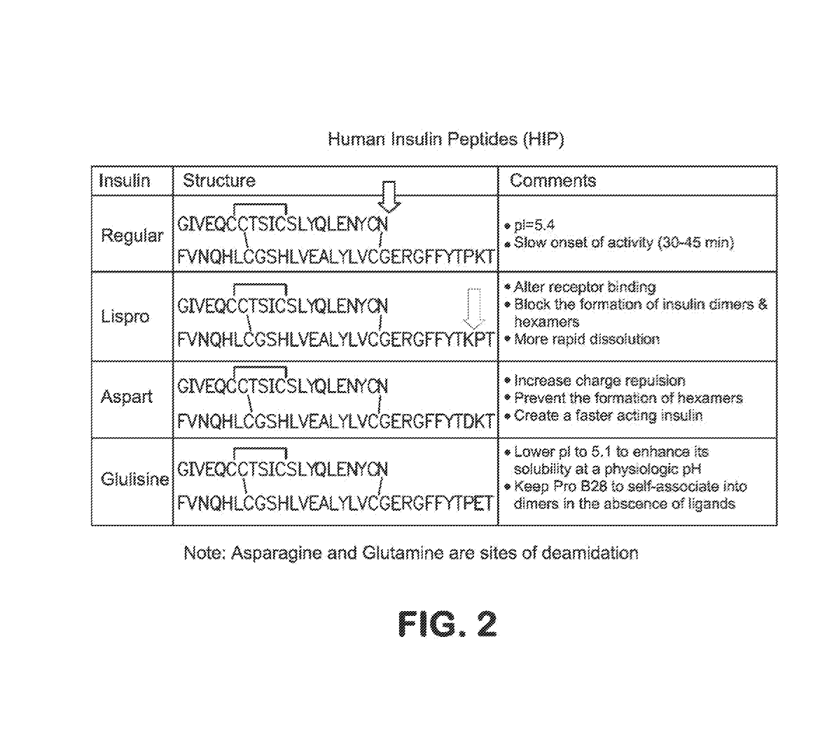

In alternative embodiments “human insulin”, or the human insulin used to practice this invention, can be the “regular” human hormone having a 21 amino acid long “A” or alpha chain GIVEQCCTSICSLYQLENYCN (SEQ ID NO:2) and a 30 amino acid long “B” or beta chain FVNQHLCGSHLVEALYLVCGERGFFYTPKT (SEQ ID NO:3) linked by two disulfide bonds at positions, as illustrated in FIG. 2. In alternative embodiments, an insulin used to practice the invention is lispro (e.g., can be HUMALOG® (Eli Lilly and Company, Indianapolis, Ind.)), which, as illustrated in FIG. 2, comprises as an alpha chain SEQ ID NO:2, with the “B” or beta chain FVNQHLCGSHLVEALYLVCGERGFFYTKPT (SEQ ID NO:4). In alternative embodiments, an insulin used to practice the invention is aspart (e.g., can be NOVOLOG® or NOVORAPID® (Novo Nordisk, Bagsværd, Denmark)), which, as illustrated in FIG. 2, comprises as an alpha chain SEQ ID NO:2, with the “B” or beta chain FVNQHLCGSHLVEALYLVCGERGFFYTDKT (SEQ ID NO:5). In alternative embodiments, an insulin used to practice the invention is glulisine (e.g., can be APIDRA™ (Sanofi S.A., Paris, France)), which, as illustrated in FIG. 2, comprises as an alpha chain SEQ ID NO:2, with the “B” or beta chain FVKQHLCGSHLVEALYLVCGERGFFYTPET (SEQ ID NO:6).

Human insulin or HIP peptides used to practice this invention can be natural sourced (e.g., isolated) or chemically or recombinantly made, e.g., as a recombinant human insulin.

In alternative embodiments, the term “human insulin peptide” (“HIP”) means a polypeptide comprising or consisting of human insulin or a human insulin analog, bioisostere, a human insulin derivative or a derivative of a human insulin analog that is rapid-acting. In alternative embodiments, a human “rapid-acting” insulin used to practice the invention is or comprises: an aspart, a NOVOLOG™ or a NOVORAPID™ (Novo Nordisk, Bagsværd, Denmark); a glulisine or an APIDRA™ (Sanofi S.A., Paris, France); a lispro, an insulin lispro protamine or a HUMALOG™ (Eli Lilly and Company, Indianapolis, Ind.); or, a human insulin, a HUMULIN R™, , a (Eli Lilly and Company, Indianapolis, Ind.) or NOVOLIN R™ (Novo Nordisk, Bagsværd, Denmark).

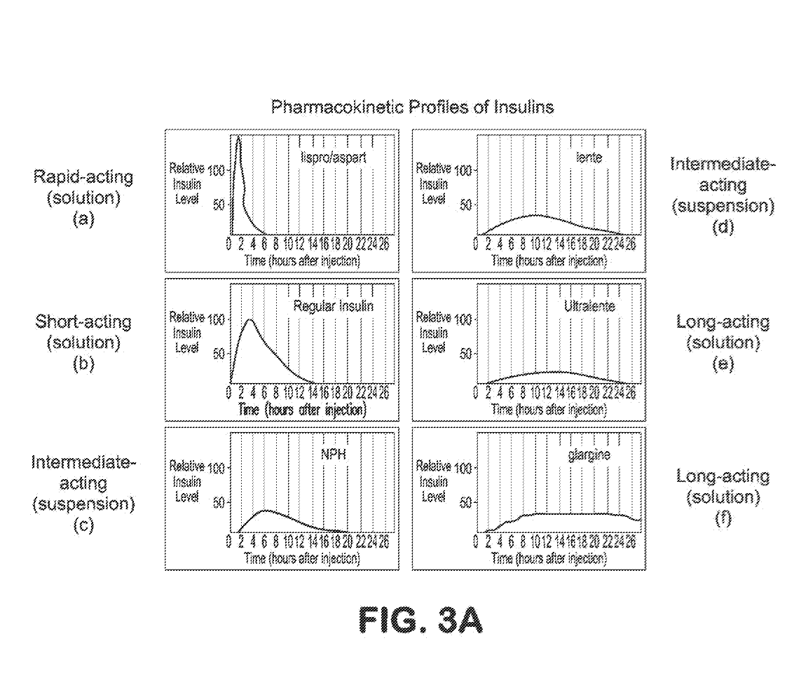

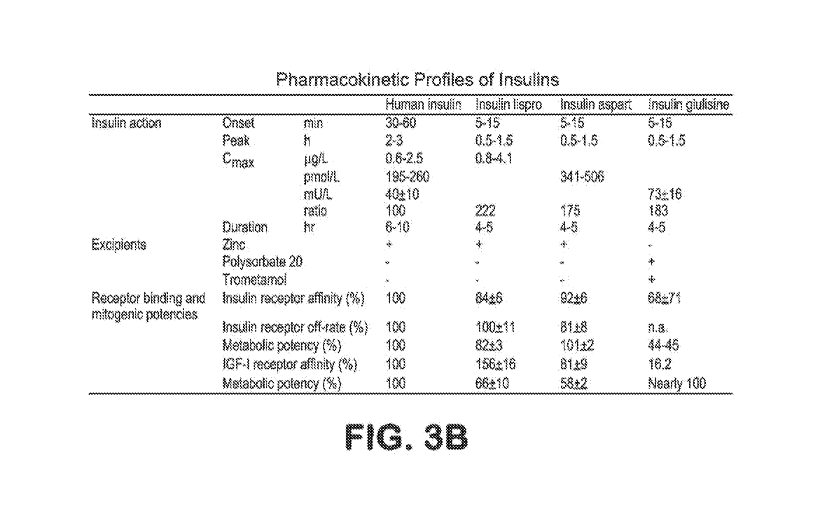

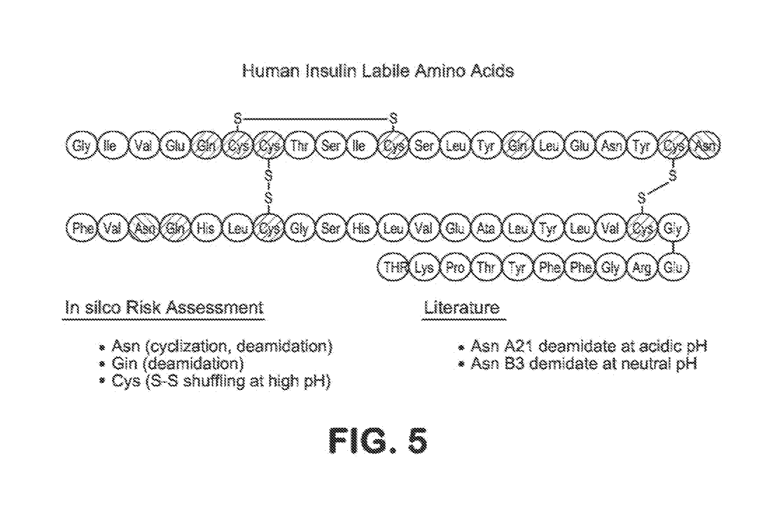

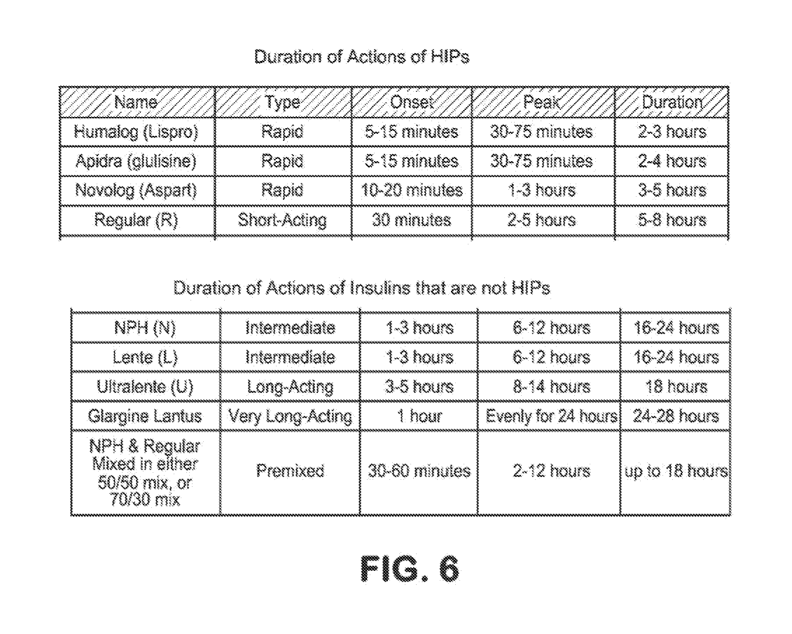

In alternative embodiments, a human “rapid-acting” insulin used to practice the invention is an insulin, insulin analog, bioisostere, or derivative having a PK profile comprising a six (6) hour maximum effective dose. In alternative embodiments, a human “rapid-acting” insulin used to practice the invention is an insulin, insulin analog, bioisostere, or derivative having a PK profile, including onset of action, duration and/or peak (e.g., as illustrated in FIG. 2), FIG. 3 (plotting duration of action against time), and/or FIG. 6, for the “rapid-acting” aspart, lispro or glulisine insulin forms. In alternative embodiments, a human “rapid-acting” insulin used to practice the invention is an insulin, insulin analog (see e.g., FIG. 5), bioisostere, or derivative having a PK profile that is substantially the same as an aspart, a NOVOLOG™ or a NOVORAPID™ (Novo Nordisk, Bagsværd, Denmark); a glulisine or an APIDRA™ (Sanofi S.A., Paris, France); a lispro, an insulin lispro protamine or a HUMALOG™ (Eli Lilly and Company, Indianapolis, Ind.); or a human insulin, HUMULIN R™, (Eli Lilly and Company, Indianapolis, Ind.) or NOVOLIN R™ (Novo Nordisk, Bagsværd, Denmark).

In alternative embodiments, a human “rapid-acting” insulin used to practice the invention is an insulin, insulin analog, bioisostere, or derivative as set forth in FIG. 3, 4 or 6. Regular insulin and all rapid-acting insulin analogs except for glulisine have labile amino acid residues Asn A21 (deamidated at acidic pH condition) and Asn B3 (deamidated at neutral pH). For glulisine, Asn B3 was replaced with Lys B3. Both lispro and aspart have same pI as regular insulin. The pI value of glulisine is only slightly lower. Thus, a diluent system (including preservative, buffer, tonicity, surfactant, and stabilizer) applicable for a regular insulin+pramlintide co-formulation can be suitable for a co-formulation of rapid-acting insulin analogs with pramlintide. In alternative embodiments, suitable excipients used in formulations of the invention are: Preservative: phenol, metacresol or combination of both; Tonicity: glycerin, mannitol, glucose or sodium chloride or any of their combinations; Surfactant: polysorbate 20 or polysorbate 80, which may be optionally absent. Exemplary formulations of the invention comprise ratios for a co-formulation of rapid-acting insulin analogs with pramlintide, and the ratios can be: Pramlintide/insulin: approximately (about) 4 mcg (μg)/1 unit (or 1 U) to approximately 24 mcg/1 unit, or any ratio between about 4 mcg (μg)/1 unit to approximately 24 mcg/1 unit.

Human insulin has “insulin activity” that can be expressed in units (U) of insulin activity, where one unit (U), or one international unit of insulin (U is equivalent to 1 U), is defined as the “biological equivalent” of 34.7 mcg (μg) pure crystalline insulin. By means of quantitative amino acid analysis of the human insulin standard (WHO, 1987), it has been found that 1 mole insulin corresponds to 1.668×10⁶ U, or that 1 U is 6 nmol (see Volume (1991) Diabetic Med 8:839).

A human insulin peptide has insulin activity. In alternative embodiments, a human insulin peptide used to practice this invention can be any rapid-acting insulin, such as a human insulin, an insulin lispro, an insulin aspart and an insulin glusiline.

In alternative embodiments, formulations of the invention comprise use of a pramlintide or a pramlintide peptide or derivatives or analogs thereof. By “pramlintide peptide” is meant any polypeptide or peptidomimetic comprising or consisting of a pramlintide, a pramlintide analog, a pramlintide derivative or a derivative of a pramlintide analog, an amino acid sequence KCNTATCATQRLANFLVHSSNNFGPILPPTNVGSNTY (SEQ ID NO:1), or physiologically acceptable salt thereof. The structural formula of pramlintide is (SEQ ID NO: 1): Lys-Cys-Asn-Thr-Ala-Thr-Cys-Ala-Thr-Gln-Arg-Leu-Ala-Asn-Phe-Leu-Val-His-Ser-Ser-Asn-Asn-Phe-Gly-Pro-Ile-Leu-Pro-Pro-Thr-Asn-Val-Gly-Ser-Asn-Thr-Tyr-NH2 with a disulfide bridge between the two Cys residues. The C-terminal carboxy group of pramlintide is amidated. In alternative embodiments, the C-terminal carboxy group of the pramlintide peptide is amidated as —NH2. In alternative embodiments, the pramlintide peptide, or a derivative or an analog thereof used to practice this invention, has at least the gastric emptying activity of a pramlintide, which can be measured in rats or humans, such as upon subcutaneous injection.

In alternative embodiments, formulations of the invention comprise use of a SYMLIN® (pramlintide acetate), or a pramlintide, which is a synthetic analog of human amylin, a naturally occurring neuroendocrine hormone synthesized by pancreatic beta cells that contributes to glucose control during the postprandial period. In alternative embodiments, formulations of the invention comprise use of a pramlintide as an acetate salt of a synthetic 37-amino acid polypeptide, which differs in amino acid sequence from human amylin by replacement with proline at positions 25 (alanine), 28 (serine), and 29 (serine).

In alternative embodiments, formulations of the invention comprise use of a pramlintide acetate as a white powder that has a molecular formula of C171H267N51O53S2.xC2H4O2 (3≦x≦8); the molecular weight is 3949.4. Pramlintide acetate is soluble in water. In alternative embodiments, the invention comprises use of a SYMLIN® formulated as a clear, isotonic, sterile solution for subcutaneous (SC) administration. In alternative embodiments, the invention comprises use of a disposable multidose SYMLINPEN® pen-injector containing 1000 mcg/mL (g/ml) of pramlintide (as an acetate), or equivalent; or the invention can use SYMLIN® vials can contain 600 mcg/mL of pramlintide (as acetate), or equivalents. Both formulations contain 2.25 mg/mL of metacresol as a preservative, D-mannitol as a tonicity modifier, and acetic acid and sodium acetate as pH modifiers. SYMLIN® has a pH of approximately 4.0.

In alternative embodiments, formulations of the invention comprise use of a human insulin or human insulin peptide, or peptidomimetic or bioisostere thereof, and optionally also comprising a physiologically acceptable salt thereof, and optionally the human insulin is or comprises: an aspart, a NOVOLOG™ or a NOVORAPID™ (Novo Nordisk, Bagsværd, Denmark); a glulisine or an APIDRA™ (Sanofi S.A., Paris, France); a lispro, an insulin lispro protamine or a HUMALOG™ (Eli Lilly and Company, Indianapolis, Ind.); or a regular (wild type) isolated or a recombinant human insulin, HUMULIN R™ (Eli Lilly and Company, Indianapolis, Ind.) or NOVOLIN R™ (Novo Nordisk, Bagsværd, Denmark).

In alternative embodiments, pramlintide and pramlintide peptide, and human insulin and human insulin peptides, used to practice this invention include any form of natural or synthetic peptide, or peptidomimetic, or biosimilar, or bioisostere, or any biologically active agonist analogue of pramlintide or human insulin having substantially the same pharmacodynamics and kinetics, e.g., substantially the same pharmacokinetic/pharmacodynamic (PK/PD) profiles, as pramlintide acetate or SYMLIN®, or a rapid-acting insulin profile, e.g. that of human insulin, lispro, aspart or glulisine, respectively. The amount of the natural or synthetic peptide, or biosimilar, or peptidomimetic, or bioisostere and the like used in a particular ratio would be the amount having the same effective in vivo activity (substantially the same pharmacokinetic/pharmacodynamic (PK/PD) profile) as the desired amount of pramlintide or human insulin. For example, if a 6 μgm or 1.52 nmoles pramlintide: 1 U (international unit) or 6.0 nmoles human insulin was desired, the amount of the natural or synthetic peptide, or peptidomimetic, or bioisostere and the like to be used in that particular ratio with 1 U (international unit) or 6.0 nmoles insulin would produce substantially the same or equivalent in vivo effect as 6 μgm or 1.52 nmoles pramlintide. The specific activity for pramlintide can be gastric emptying activity, optionally measured in a rat, or optionally in a human. The molar amount of human insulin can be replaced by the molar amount of human insulin peptide, e.g. lispro, aspart, glulisine, that provides the same units of insulin activity as would be known in the field. For example, for the ratio of 6 microgram or 1.52 nanomoles of pramlintide to one unit (1 U) (international unit) of human insulin, the units of the human insulin peptide would be identical to the stated units of human insulin. However, for the ratio expressed in moles (or nanomoles) of human insulin that provide the given units of insulin activity, the equivalent mole of human insulin peptide is that number of mole of HIP that provides that same number of units of insulin activity.

In alternative embodiments, a pramlintide used to practice this invention is as described in U.S. Pat. No. 7,312,196.

In alternative embodiments, a pramlintide polypeptide or peptide used to practice this invention includes polypeptides having at least about 85%, 86%, 87%, 88%, 89%, 90%, 91%, 92%, 93%, 94%, 95%, 96%, 97%, 98%, 99%, or more sequence identity to SEQ ID NO: 1, wherein this pramlintide polypeptide or peptide embodiment has substantially the same pharmacodynamics and kinetics, e.g., substantially the same pharmacokinetic/pharmacodynamic (PK/PD) profiles, as pramlintide acetate or SYMLIN®. In alternative embodiments, an insulin polypeptide or peptide used to practice this invention includes polypeptides having at least about 85%, 86%, 87%, 88%, 89%, 90%, 91%, 92%, 93%, 94%, 95%, 96%, 97%, 98%, 99%, or more sequence identity to SEQ ID NO:2 and/or SEQ ID NO:3 (the 21 amino acid long insulin “A” or alpha chain is SEQ ID NO:2) and the 30 amino acid long insulin “B” or beta chain is SEQ ID NO:3), wherein this insulin embodiment (the biologically active insulin as an A and B chain) has substantially the same pharmacodynamics and kinetics, e.g., substantially the same pharmacokinetic/pharmacodynamic (PK/PD) profiles, as that of human insulin, lispro, aspart or glulisine.

In alternative embodiments, formulations of the invention comprise use of a pramlintide salt, e.g., salts with various inorganic and organic acids and bases, for example, HCl, HBr, H₂SO₄, H₃PO₄, trifluoroacetic acid, acetic acid, formic acid, methanesulfonic acid, toluenesulfonic acid, maleic acid, fumaric acid and/or camphorsulfonic acid. Salts can prepared with bases, for example, ammonium salts, alkali metal salts (such as sodium and potassium salts) and alkali earth salts (such as calcium and magnesium salts). The salts may be formed by conventional means, as by reacting the free acid or base forms of the product with one or more equivalents of the appropriate base or acid in a solvent or medium in which the salt is insoluble, or in a solvent such as water which is then removed in vacuo or by freeze-drying or by exchanging the ions of an existing salt for another ion on a suitable ion exchange resin.

In alternative embodiments, formulations of the invention comprise use of various stereoisomers of pramlintide, e.g., where the chiral centers on the peptide backbone are all S.

Pramlintides used to practice the invention can be prepared by those of ordinary skill in the art, as described in “Amylin Agonist Peptides and Uses Therefor,” U.S. Pat. No. 5,686,411. In one embodiment, “human amylin” is meant the 37 amino acid amylin set forth in U.S. Pat. No. 5,357,052.

Pramlintides used to practice the invention can be formulated into stable and safe pharmaceutical compositions for administration. In various embodiments, pramlintides used to practice the invention are alternatively formulated as: (1) liquid pramlintide formulations for mixing, (2) dried (e.g., powder) pramlintide formulations for mixing, and (3) co-formulations comprising pramlintide and insulin as a product of mixing: two liquid “starting” or “remix” pramlintide and insulin formulations; a dried pramlintide formulation and a liquid insulin formulation; a liquid pramlintide formulation and a dried insulin formulation; or a liquid co-formulation made by adding a diluent to a dried pramlintide and a dried insulin formulation.

In various embodiments, pramlintides formulations comprise a buffer, preferably a non-volatile buffer for the dried form, and can be alternatively formulated at approximately 0.01 to 1.0% (w/v), or 0.05 to 1.0%, or approximately 0.02 to 0.5% (w/v) of an acetate, phosphate, citrate, tartrate or glutamate buffer allowing a pH of the final composition of from about 3.0 to about 7.0. In alternative embodiments, the stability of a peptide formulation can be enhanced by maintaining the pH of the formulation in the range of about 3.0 to about 7.0 when in liquid form. In alternative embodiments, the pH of the formulation is maintained in the range of about 3.5 to 5.0, or about 3.5 to 6.5, or from about 3.7 to 4.3 or 4.0, or about 3.8 to 4.2 or 4.0, or from about 3.7 to 4.3 or 4.35, or about 3.8 to 4.2 or 4.3, or the pH may be about 4.0, 4.1 or 4.2 or 4.3.

In alternative embodiments, a buffer used to practice the invention is an acetate or an equivalent buffer, e.g., at a final formulation concentration of from about 1 to 5 to about 60 mM; or a phosphate buffer, e.g., at a final formulation concentration of from about 1 to 5 to about to about 30 mM; or a glutamate buffer, e.g., at a final formulation concentration of from about 1 to 5 to about to about 60 mM, or, the buffer can be an acetate buffer, e.g., at a final formulation concentration of from about 5 to about 30 mM.

In alternative embodiments, a formulation of the invention can comprise approximately 1.0 to 10% (w/v) of a carbohydrate or a polyhydric alcohol tonicifier and, optionally, approximately 0.005 to 1.0% (w/v) of a preservative selected from the group consisting of m-cresol, benzyl alcohol, methyl, ethyl, propyl and butyl parabens and phenol.

Pramlintides used to practice the invention be formulated at a range of concentrations, e.g., between about 0.01% to about 98% w/w, or between about 1 to about 98% w/w, or between 80% and 90% w/w, or between about 0.01% to about 50% w/w, or between about 10% to about 25% w/w. A sufficient amount of water for injection may be used to obtain the desired concentration of solution.

Pramlintides used to practice the invention can be formulated with additional tonicifying agents such as sodium chloride, as well as other known excipients. In alternative embodiments, excipients can maintain the overall tonicity of the pramlintide. In alternative embodiments, an excipient is included at various concentrations, for example, at a concentration range from about between about 0.02% to about 20% w/w, between about 0.02% and 0.5% w/w, about 0.02% to about 10% w/w, or about 1% to about 20% w/w. In alternative embodiments, an excipient may be included in solid (including powdered), liquid, semi-solid or gel form.

In alternative embodiments a stabilizer is included in a formulation, but it is not necessarily needed. If included, however, exemplary stabilizers can be a carbohydrate or a polyhydric alcohol. In alternative embodiments stabilizers can be approximately 1.0 to 10% (w/v) of a carbohydrate or polyhydric alcohol. In alternative embodiments the polyhydric alcohols can include such compounds as sorbitol, mannitol, glycerol, and polyethylene glycols (PEGs); the carbohydrates can be mannose, ribose, sucrose, fructose, trehalose, maltose, inositol, and lactose, or any cyclic molecule that may contain a keto or aldehyde group; or, include carbohydrates such as galactose, arabinose, lactose or any other carbohydrate which does not have an adverse effect on a diabetic patient, i.e., the carbohydrate is not metabolized to form unacceptably large concentrations of glucose in the blood. Such carbohydrates are well known in the art as suitable for diabetics. Sucrose and fructose are suitable for use with amylins in non-diabetic applications, e.g. treating obesity. In alternative embodiments stabilizers can be 0.1 to 5% (w/v) of an amino acid. In alternative embodiments the amino acids can include glycine, methionine, histidine, and arginine.

In alternative embodiments, if a stabilizer is included, a peptide used to practice the invention is stabilized with a polyhydric alcohol such as sorbitol, mannitol, inositol, glycerol, xylitol, and polypropylene/ethylene glycol copolymer, as well as various polyethylene glycols (PEG) of molecular weight 200, 400, 1450, 3350, 4000, 6000, and 8000). In alternative embodiments lyophilized formulations of the present invention can maintain tonicity with the same formulation component that serves to maintain their stability, e.g., a mannitol as the polyhydric alcohol can be used for this purpose.

In alternative embodiments preservative are used, e.g., in a range from 0.005 to 1.0% (w/v); or, for each preservative, alone or in combination with others: benzyl alcohol (0.1-1.0%), or m-cresol (0.1-0.6%), or phenol (0.1-0.8%) or combination of methyl (0.05-0.25%) and ethyl or propyl or butyl (0.005%-0.03%) parabens. The parabens can be lower alkyl esters of para-hydroxybenzoic acid.

In alternative embodiments it may also be desirable to add sodium chloride or other salt to adjust the tonicity of the pharmaceutical formulation, depending on the tonicifier selected. In alternative embodiments, this depends on the particular formulation selected. In alternative embodiments parenteral formulations must be isotonic or substantially isotonic, otherwise significant irritation and pain may occur at the site of administration.

In alternative embodiments, for parenteral products, a vehicle is water, e.g., a water of suitable quality for a parenteral administration, e.g., prepared either by distillation or by reverse osmosis. Water for injection may be an aqueous vehicle for use in the pharmaceutical formulation of the present invention.

In alternative embodiments other ingredients are present in a pharmaceutical formulation of the present invention. Such additional ingredients may include, e.g., wetting agents, emulsifiers, oils, antioxidants, bulking agents, tonicity modifiers, chelating agents, metal ions, oleaginous vehicles, proteins (e.g., human serum albumin, gelatin or proteins) and a zwitterion (e.g., an amino acid such as betaine, taurine, arginine, glycine, lysine and histidine). In alternative embodiments, polymer solutions, or mixtures with polymers can provide an opportunity for controlled release of a peptide. Such additional ingredients should not adversely affect the overall stability of the pharmaceutical formulation of the present invention.

In alternative embodiments, containers are also an integral part of a formulation of an injection and may be considered a component, for there is no container that is totally inert, or does not in some way affect the liquid it contains, particularly if the liquid is aqueous. Therefore, the selection of a container for a particular injection must be based on a consideration of the composition of the container, as well as of the solution, and the treatment to which it will be subjected. Adsorption of the peptide to the glass surface of the vial can also be minimized, if necessary, by use of borosilicate glass, for example, Wheaton Type I borosilicate glass #33 (Wheaton Type 1-33) or its equivalent (Wheaton Glass Co.). Other vendors of similar borosilicate glass vials and cartridges acceptable for manufacture include Kimbel Glass Co., West Co., Bunder Glas GMBH and Forma Vitrum.

In order to permit introduction of a needle from a hypodermic syringe into a multiple-dose vial and provide for resealing as soon as the needle is withdrawn, the open end of each vial can be sealed with a rubber stopper closure held in place by an aluminum band. Stoppers for glass vials, such as, West 4416/50, 4416/50 (Teflon faced) and 4406/40, Abbott 5139 or any equivalent stopper can be used as the closure for pharmaceutical for injection. These stoppers are compatible with the peptide as well as the other components of the formulation. In alternative embodiments peptides can be lyophilized into vials, syringes or cartridges for subsequent reconstitution. Liquid formulations of the present invention can be filled into one or two chambered cartridges, or one or two chamber syringes.

Insulins used to practice the invention can be formulated into stable and safe pharmaceutical compositions for administration. In various embodiments, insulins used to practice the invention are alternatively formulated as: (1) liquid insulin formulations for mixing, including from commercially available sources (2) dried (e.g., powder) insulin formulations for mixing, and (3) co-formulations comprising pramlintide and insulin as a product of mixing: two liquid “starting” or “remix” pramlintide and insulin formulations; a dried pramlintide formulation and a liquid insulin formulation; a liquid pramlintide formulation and a dried insulin formulation; or a liquid co-formulation made by adding a diluent to a dried pramlintide and a dried insulin formulation.

In alternative embodiments, any human insulin can be used to practice this invention. For example, a crystalline insulin can be prepared by the precipitation of the hormone in the presence of zinc (as zinc chloride) in a suitable buffer medium. Crystalline insulin when dissolved in water is also known as regular insulin. Following subcutaneous injection it is rapidly absorbed (15-60 minutes). Its action is prompt in onset and relatively short in duration, i.e., it reaches its peak effect in about 1.5 to 4 hours, and lasts for about 5-9 hours.

In alternative embodiments, a liquid or reconstitutable dried pharmaceutical composition or formulation of the invention comprises a human insulin complexed with a zinc or a Zn⁺², e.g., where a human insulin is complexed with a zinc in a ratio of molar ratio of at least 6:2, or, the human insulin is complexed with the zinc and is substantially hexameric, or the human insulin is complexed with the zinc and the insulin is greater than about 95%, 96%, 97%, 98%, 99% or more hexameric, or is between about 90% and 100% hexameric. PK depends on insulin absorption rate, which depends on insulin structure when injected. In alternative embodiments, insulin structure is a hexamer held together by two zincs. A large hexamer, e.g., 32.5 KDa, absorbs more slowly than dissociated dimer or monomer forms; so, in alternative embodiments, you want to maintain a hexamer state of insulin in a formulation, thus, zinc is needed. As sample dilutes or zinc is chelated by fluids under the skin, the hexamer dissociates to dimer or monomer, which smaller forms absorb. The larger hexamer may absorb directly, but this is not the major path of absorption. A formulation with no zinc would be predominately dimer/monomer and have rapid absorption.

Any order of mixing or introduction of pramlintide and insulin into a device or product of manufacture, e.g., an insulin pump, pen or a syringe, can be used, such as simultaneous or concerted and sequential admixing. In one embodiment, the order is to place the insulin in the device or product of manufacture (insulin pump, pen or syringe) first, followed by pramlintide.

The amount of insulin and pramlintide formulation, e.g., the ratio of pramlintide to insulin used, depends on the individual needs of a particular patient. The pharmaceutical formulations of the present invention can be administered to any human or mammal in need of such treatment.

In alternative embodiments, regular insulin products may be mixed with pramlintide at a pH of approximately 4.0, 4.1, 4.2 or 4.3 with about 20 or 30 mM acetate buffer to maintain the solubility of the insulin. The pH of the mixture would then be less than 4.4. In one embodiment, a pramlintide formulation has a high buffer capacity (e.g., with 30 mM acetate) and at low label strength, e.g., 0.1 mg/ml, and forms a clear solution instantaneously (under a minute) when mixed with regular insulin products in the range of five to 20 units. This low label strength of pramlintide results in a high dose volume, 300 μL. This increased volume may be advantageous for bringing the pH down to less than 4.4 almost immediately by increasing insulin dilution factor and facilitating the transition of insulin from hexamer to monomer before injection. This modulation of insulin can be advantageous in permitting increased rate of absorption and causing rapid-time action without affecting bioavailability of insulin.

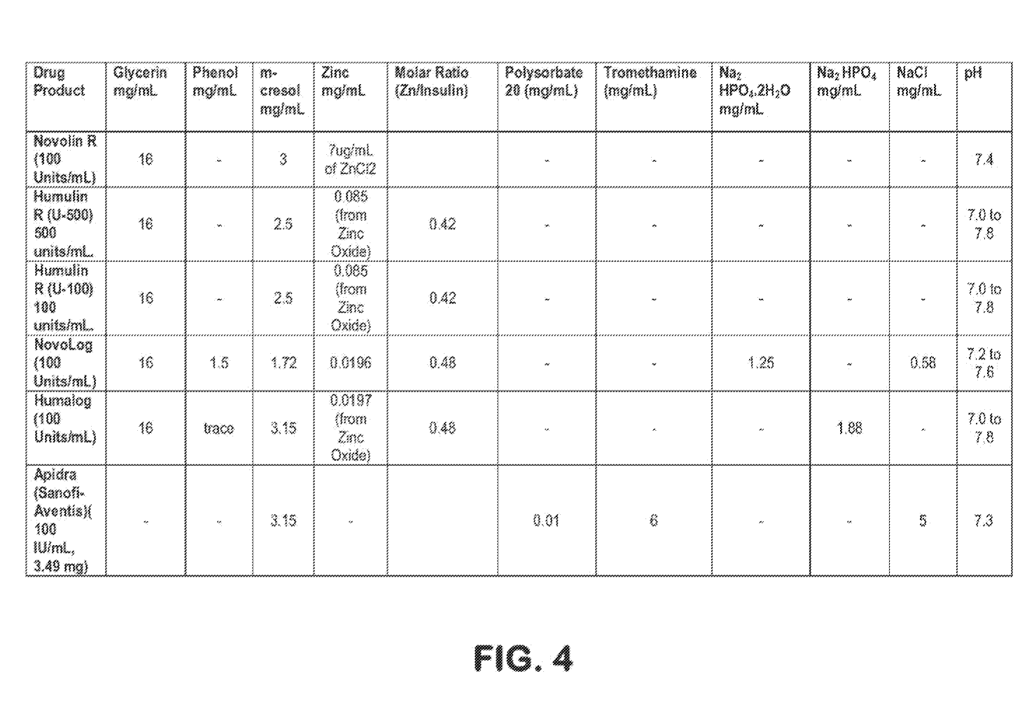

In one embodiment, peptide formulations of pramlintide are at a pH of approximately 4.0, 4.1, 4.2 or 4.3, or are at a pH from 3.7 to 4.4; and 2 to 30 mM buffer concentration and high potency. In alternative embodiments these are mixed with regular insulin products before injection to yield solutions with a pH greater than about 6.8 so that the properties of insulin are not affected. These mixtures of the invention will not affect the rate of absorption or bioavailability of insulin nor the bioavailability of the peptide. FIG. 4 describes alternative exemplary formulations of the invention.

In alternative embodiments, the pharmaceutical compositions used to practice the methods of the invention can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration are well described in the scientific and patent literature, see, e.g., the latest edition of Remington's Pharmaceutical Sciences, Maack Publishing Co, Easton Pa. (“Remington's”).

Therapeutic agents used to practice the methods of the invention can be administered alone or as a component of a pharmaceutical formulation (composition). The compounds may be formulated for administration in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

While pramlintide or insulin formulations and/or P:I co-formulations used to practice the invention are designed for subcutaneous (SC) or subdermal administration, in alternative embodiments, the invention can also comprise use of alternative delivery methods as an alternative to or in supplement to SC administration, including formulations for intravenous (IV), oral/nasal, topical, parenteral, rectal, and/or intravaginal administration.

Formulations used to practice the invention can be presented in unit dosage form and may be prepared by any methods known in the art of pharmacy and for the manufacture of pharmaceuticals. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect.

Formulations used to practice the invention can comprise sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Formulations used to practice the invention can be aqueous suspensions, and can contain insulin or pramlintide in admixture with excipients suitable for the manufacture of aqueous suspensions. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

In practicing this invention, the pharmaceutical compounds can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In practicing this invention, the pharmaceutical compounds can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

### Dried and Lyophilized Formulations

The pharmaceutical compounds and formulations used to practice the methods of the invention can be lyophilized. The invention provides a stable lyophilized formulation comprising a composition of the invention, which can be made by lyophilizing a solution comprising a pharmaceutical of the invention and a bulking agent, e.g., mannitol, glycine, trehalose, raffinose, and sucrose or mixtures thereof.

Compositions used to practice this invention include liquid and reconstitutable dried formulations, and separate formulations of pramlintide and insulin. For example, in one embodiment, separate formulations can comprise commercial formulations, e.g., human insulin, lispro, aspart and/or glulisine, that can be: (1) mixed to yield a co-formulation at desired ratio, a ratio of pramlintide and insulin of the invention, or, (2) unmixed but co-delivered at a desired ratio, a ratio of pramlintide and insulin of the invention. In alternative embodiments, SYMLIN™ formulation(s) of pramlintide acetate are mixed with a human insulin formulation. Co-Formulations can be liquid or reconstitutable dried, and may have 1 day to 3 day or to 3-month in-use periods.

### Nanoparticles and Liposomes

The invention also provides nanoparticles and liposomal membranes comprising compounds used to practice the methods of the invention.

The invention also provides nanocells to allow the sequential delivery of two different therapeutic agents with different modes of action or different pharmacokinetics, at least one of which comprises a composition used to practice a method of the invention. A nanocell is formed by encapsulating a nanocore with a first agent inside a lipid vesicle containing a second agent; see, e.g., Sengupta, et al., U.S. Pat. Pub. No. 20050266067. The agent in the outer lipid compartment is released first and may exert its effect before the agent in the nanocore is released. The nanocell delivery system may be formulated in any pharmaceutical composition for delivery to patients suffering from a diseases or condition as described herein, e.g., a diabetes, a dementia, Alzheimer's disease, postprandial or reactive hypoglycemia or an insulin resistance, a PolyCystic Ovary Syndrome (PCOS), a ketoacidosis, a gestational diabetes, a hyperkalemia, a cancer or a cachexia, a beta blocker overdose, a jaundice, a cancer, septic shock, an infection, a fever, pain and related symptoms or conditions, and the like.

The invention also provides multilayered liposomes comprising compounds used to practice this invention, e.g., for transdermal absorption, e.g., as described in Park, et al., U.S. Pat. Pub. No. 20070082042. The multilayered liposomes can be prepared using a mixture of oil-phase components comprising squalane, sterols, ceramides, neutral lipids or oils, fatty acids and lecithins, to about 200 to 5000 nm in particle size, to entrap a composition of this invention.

A multilayered liposome used to practice the invention may further include an antiseptic, an antioxidant, a stabilizer, a thickener, and the like to improve stability. Synthetic and natural antiseptics can be used, e.g., in an amount of 0.01% to 20%. Antioxidants can be used, e.g., BHT, erysorbate, tocopherol, astaxanthin, vegetable flavonoid, and derivatives thereof, or a plant-derived antioxidizing substance. A stabilizer can be used to stabilize liposome structure, e.g., polyols and sugars. Exemplary polyols include butylene glycol, polyethylene glycol, propylene glycol, dipropylene glycol and ethyl carbitol; examples of sugars are trehalose, sucrose, mannitol, sorbitol and chitosan, or a monosaccharides or an oligosaccharides, or a high molecular weight starch. A thickener can be used for improving the dispersion stability of constructed liposomes in water, e.g., a natural thickener or an acrylamide, or a synthetic polymeric thickener. Exemplary thickeners include natural polymers, such as acacia gum, xanthan gum, gellan gum, locust bean gum and starch, cellulose derivatives, such as hydroxy ethylcellulose, hydroxypropyl cellulose and carboxymethyl cellulose, synthetic polymers, such as polyacrylic acid, poly-acrylamide or polyvinylpyrollidone and polyvinylalcohol, and copolymers thereof or cross-linked materials.

Liposomes can be made using any method, e.g., as described in Park, et al., U.S. Pat. Pub. No. 20070042031, including method of producing a liposome by encapsulating a therapeutic product comprising providing an aqueous solution in a first reservoir; providing an organic lipid solution in a second reservoir, wherein one of the aqueous solution and the organic lipid solution includes a therapeutic product; mixing the aqueous solution with said organic lipid solution in a first mixing region to produce a liposome solution, wherein the organic lipid solution mixes with said aqueous solution so as to substantially instantaneously produce a liposome encapsulating the therapeutic product; and immediately thereafter mixing the liposome solution with a buffer solution to produce a diluted liposome solution.

The compositions and formulations used to practice the invention can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587. For example, in one embodiment, compositions and formulations used to practice the invention are delivered by the use of liposomes having rigid lipids having head groups and hydrophobic tails, e.g., as using a polyethyleneglycol-linked lipid having a side chain matching at least a portion the lipid, as described e.g., in US Pat App Pub No. 20080089928. In another embodiment, compositions and formulations used to practice the invention are delivered by the use of amphoteric liposomes comprising a mixture of lipids, e.g., a mixture comprising a cationic amphiphile, an anionic amphiphile and/or neutral amphiphiles, as described e.g., in US Pat App Pub No. 20080088046, or 20080031937. In another embodiment, compositions and formulations used to practice the invention are delivered by the use of liposomes comprising a polyalkylene glycol moiety bonded through a thioether group and an antibody also bonded through a thioether group to the liposome, as described e.g., in US Pat App Pub No. 20080014255. In another embodiment, compositions and formulations used to practice the invention are delivered by the use of liposomes comprising glycerides, glycerophospholipides, glycerophosphinolipids, glycerophosphonolipids, sulfolipids, sphingolipids, phospholipids, isoprenolides, steroids, stearines, sterols and/or carbohydrate containing lipids, as described e.g., in US Pat App Pub No. 20070148220.

The invention also provides nanoparticles comprising compounds used to practice this invention to deliver a composition of the invention as a drug-containing nanoparticles (e.g., a secondary nanoparticle), as described, e.g., in U.S. Pat. Pub. No. 20070077286. In one embodiment, the invention provides nanoparticles comprising a fat-soluble drug of this invention or a fat-solubilized water-soluble drug to act with a bivalent or trivalent metal salt.

### Therapeutically Effective Amount and Doses

The amount of pharmaceutical composition adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration. The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate.

Single or multiple administrations of formulations can be given depending on the dosage and frequency as required and tolerated by the patient. The formulations should provide a sufficient quantity of active agent to effectively treat, prevent or ameliorate a conditions, diseases or symptoms as described herein. For example, an exemplary pharmaceutical formulation administration into the blood stream, into a body cavity or into a lumen of an organ, e.g., intravenously (IV), is 6 μgm or 1.52 nmoles pramlintide: 1 U (international unit) or 6.0 nmoles insulin, or about 0.25 mole pramlintide to 1 mole insulin (6 μgm:1 U), or 3.95 mole insulin to 1 mole pramlintide. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington's, supra.

The methods of the invention can further comprise co-administration with other drugs or pharmaceuticals, e.g., compositions drugs or pharmaceuticals for treating or ameliorating a diabetes, a dementia, Alzheimer's disease, postprandial or reactive hypoglycemia or an insulin resistance, a PolyCystic Ovary Syndrome (PCOS), a ketoacidosis, a gestational diabetes, a hyperkalemia, a cancer or a cachexia, a beta blocker overdose, a jaundice, a cancer, septic shock, an infection, a fever, pain and related symptoms or conditions, and the like. In alternative embodiments, the other provided drugs are those useful to treat or ameliorate or for overcoming or diminishing or preventing a prediabetes, a gestational diabetes, a Type 1 or a Type 2 diabetes, or an abnormality of blood glucose control, or inability to control blood glucose, or an elevation of fasting glucose or Impaired Fasting Glucose (IFG), or an abnormality of tolerance to a glucose load or Impaired Glucose Tolerance (IGT), or a hyperglycemia induced by an illness, a trauma, a medication administration or a form of metabolic, psychological or physical stress, or a hyperglycemia induced by steroids (steroid-induced diabetes), latent autoimmune diabetes in adults (LADA), or a postprandial or reactive hypoglycemia or an insulin resistance. For example, the methods and/or compositions and formulations of the invention can be co-formulated with and/or co-administered with antibiotics (e.g., antibacterial or bacteriostatic peptides or proteins), particularly those effective against gram negative bacteria, fluids, cytokines, immunoregulatory agents, anti-inflammatory agents, complement activating agents, such as peptides or proteins comprising collagen-like domains or fibrinogen-like domains (e.g., a ficolin), carbohydrate-binding domains, and the like and combinations thereof.

In alternative embodiments compositions of the invention, and methods of the invention, can replace any rapid-acting insulin formulation or use. In alternative embodiments, compounds, compositions, pharmaceutical compositions and formulations used to practice the invention can be administered for prophylactic and/or therapeutic treatments; for example, the invention provides compositions and methods for overcoming or diminishing or preventing a diabetes, a dementia, Alzheimer's disease, postprandial or reactive Hypoglycemia or an insulin resistance, a PolyCystic Ovary Syndrome (PCOS), a ketoacidosis, a hyperkalemia, a cancer or a cachexia, a beta blocker overdose, a jaundice, septic shock, an infection, a fever, pain and related symptoms or conditions, and the like. In alternative embodiments, the invention provides compositions and methods for overcoming or diminishing or preventing a prediabetes, a gestational diabetes, a Type 1 or a Type 2 diabetes, or an abnormality of blood glucose control, or inability to control blood glucose, or an elevation of fasting glucose or Impaired Fasting Glucose (IFG), or an abnormality of tolerance to a glucose load or Impaired Glucose Tolerance (IGT), or a hyperglycemia induced by an illness, a trauma, a medication administration or a form of metabolic, psychological or physical stress, or a hyperglycemia induced by steroids (steroid-induced diabetes), a latent autoimmune diabetes in adults (LADA), or a postprandial or reactive Hypoglycemia or an insulin resistance.

In therapeutic applications, compositions are administered to a subject already suffering from a condition, infection or disease in an amount sufficient to cure, alleviate or partially arrest the clinical manifestations of the condition, infection or disease (e.g., disease or condition associated with dysfunctional blood glucose control) and its complications (a “therapeutically effective amount”). In the methods of the invention, a pharmaceutical composition is administered in an amount sufficient to treat, slow or reverse the progress of or moderate the symptoms or side effects of (all covered by “ameliorate”) or prevent a disease or condition associated with dysfunctional blood glucose control. The amount of pharmaceutical composition adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

In alternative embodiments, the invention provides compositions and methods using, or for replacing, a rapid-acting insulin. Replacement can initially be based on an insulin unit for unit basis. For example, the units of human insulin, e.g. as HUMULIN™, normally taken or prescribed for the patient, are replaced by an amount of the composition of the invention (that can have an optimized ratio of pramlintide or pramlintide peptide and insulin or human insulin peptide (HIP)) that provides the same number of insulin units. In alternative embodiments, for pre-meal administration, e.g. for treating diabetes, the number of insulin units is typically calculated based on the amount of calculated carbohydrate intake of the patient. In alternative embodiments, for delivering a basal level of insulin activity, e.g. for treating diabetes, the number of units per day is typically calculated by the physician using algorithms known in the art.

For either continuous infusion, bolus, basal or bolus/basal delivery of the formulations of the invention, the amount provided or administered to the individual is that amount which provides the same number of units of insulin as would be taken if the pramlintide was not taken into account. In alternative embodiments, the amount of insulin units administered or prescribed are reduced by about 1%, 2%, 3%, 4%, 5%, 10%, 15%, 16%, 17%, 18%, 19%, 20%, 21%, 22%, 23%, 24%, 25%, 30%, 40% or 50% or more, or reduced between about 1% and 50%, or reduced between about 15% and 25%, from the amount of insulin units calculated for an insulin composition that does not contain pramlintide or pramlintide peptide. In other words, if the clinician or subject calculates the need for 10 units of insulin, the amount of a formulation of the invention (an optimized ratio of the invention) needed as replacement can be that amount that provides 10, 9.5, 9, 8.5, 8, 7.5, 7 or 5 or less insulin units, or 1%, 2%, 3%, 4%, 5%, 10%, 15%, 20%, 25%, 30%, 40% or 50% or more fewer insulin units. For example, in one exemplary embodiment, insulin or insulin boluses administered to an individual can be reduced by approximately 21% at a P/I ratio of about 9 μg/U (or 9 μg pramlintide or pramlintide peptide to 1 U human insulin or human insulin peptide (HIP)) to account for the in vivo effects of pramlintide and to avoid a postprandial hypoglycaemia.

Any formulation can be used to determine what the insulin alone concentration is appropriate for any particular individual, e.g., for patients who aren't on an insulin regimen at home, many experts offer these rules of thumb for estimating total daily dose: 0.3 units/kg/day for patients who are lean, on hemodialysis, frail and elderly, insulin-sensitive, or at risk for hypoglycemia; 0.4 units/kg/day for a patient at normal weight; 0.5 units/kg/day for overweight patients; and 0.6 or more units/kg/day or more for patients who are obese, on high-dose steroids or insulin-resistant. Individual insulin doses are also individually adjusted for the amount and type of food that is consumed, the amount of exercise and a whole host of other factors.

### Products of Manufacture, Insulin Pumps, Devices

In alternative embodiments, the invention provides insulin pumps, devices, subcutaneous insulin infusion therapy devices, continuous subcutaneous insulin infusion therapy devices, infusion therapy devices, reservoirs, ampoules, vials, syringes, cartridges, disposable pen or jet injectors, prefilled pens or syringes or cartridges, cartridge or disposable pen or jet injectors, two chambered or multi-chambered pumps, syringes, cartridges or pens or jet injectors, or an artificial pancreas, comprising a formulation having an insulin:pramlintide ratio of the invention.

In alternative embodiments, the invention comprises use of a two or multi-chambered device, e.g., a pen, compartment, vial, cartridge or syringe. In one embodiment, pramlintide and insulin are loaded or filled into the device in separate chambers, sections, vials and the like, and the device allows for mixing or reconstitution of a final P:I formulation at a set or preset ratio to be administered. In one embodiment, the device allows for changing the amount of insulin to be administered at any particular time, but keeping a preset, or an adjustable setting, of the P:I ratio. In one embodiment, the device can automatically, e.g., by a computer implemented method operably connected to the device, determine the amount of pramlintide needed to be added to a desired dosage of insulin to keep the final administered formulation at a set or preset P:I ratio. In one embodiment, the device can read a patient's blood sugar, then automatically determine the amount of pramlintide needed to be added to a desired dosage of insulin to keep the final administered formulation at a set or preset P:I ratio, and the amount of final formulation to be delivered to the patient.

In one embodiment, pramlintide and/or insulin are loaded during manufacturing into one chamber, vial, compartment, cartridge, pen or syringe chamber, e.g., in contact with a plunger or equivalent to mix and/or administer a final formulation to a patient. In alternative embodiments, a divider can be a rubber or another suitable material known in the art. In alternative embodiments, a divider isolates a solution of a peptide contained in the first chamber from another peptide a second chamber. For example, prior to administration, the needed amount of insulin can be measured (by individual or automated) into a second chamber. In alternative embodiments, the insulin is measured into the two or multi-chambered cartridge, pen, syringe and the like, immediately prior to administration to a subject. When both chambers are filled with the appropriate amount of pramlintide and insulin, the two chambers may be administered to a subject, together (mixed in the device) or in series (not mixed in the device). The invention provides devices that can be set to administer the desired P:I ratio, or can by computer implemented method determine the desired P:I ratio (e.g., depending on the amount of insulin needed at a particular time) and administer the desired P:I ratio, where the pramlintide and insulin are not mixed in the device by are injected (e.g., subcutaneously) separately. Alternatively, the device can mix the pramlintide and insulin solutions.

Another alternative embodiment, a device of the invention stores and formulates, or formulates, insulin and pramlintide at a desired P:I ratio for use with insulin pumps or similar devices. Formulations of pramlintide can be filled into cartridges or syringes or other devices that allow the user of an insulin pump to co-administer the peptide as needed.

In alternative embodiments, insulin pumps used to practice the invention are small devices, e.g., about the size of a small cell phone, that can be worn externally and can be discreetly clipped to a belt, slipped into a pocket, or hidden under clothes. It delivers precise doses of rapid-acting insulin to closely match individual needs. In alternative embodiments, the insulin pump holds a cartridge (reservoir) of insulin and/or pramlintide, or a pramlintide:insulin formulation at a ratio of the invention, that delivers the formulation through an infusion set. In alternative embodiments the infusion set comprises tubing that connects to a reservoir, and second tube, or the cannula; the cannula is inserted with a small needle that is removed once inserted. Before starting on the insulin pump, the device needs to be given instructions to deliver the proper amount of insulin and/or pramlintide, or a pramlintide:insulin formulation at a ratio of the invention; or in alternative embodiments, this can be determined by a computer and computer-implemented method of the invention.

In alternative embodiments, devices used to practice the invention, e.g., an insulin pump, comprise use of a Continuous Glucose Monitoring (CGM) to measure glucose values continuously over time through the sensor inserted under the skin. The glucose sensor can be an electrode inserted under the skin that measures glucose levels in the fluid within the skin; and it produces an electronic signal that is related to the amount of glucose present in the blood. The glucose sensor can be connected to a transmitter that sends the information to the data monitoring device using radio frequency. In alternative embodiments the monitor displays the glucose reading on its screen and notifies if it detects the individual, or a computer of this invention, that glucose is reaching a high or low limit. CGM systems can alert before reaching a glucose limit.

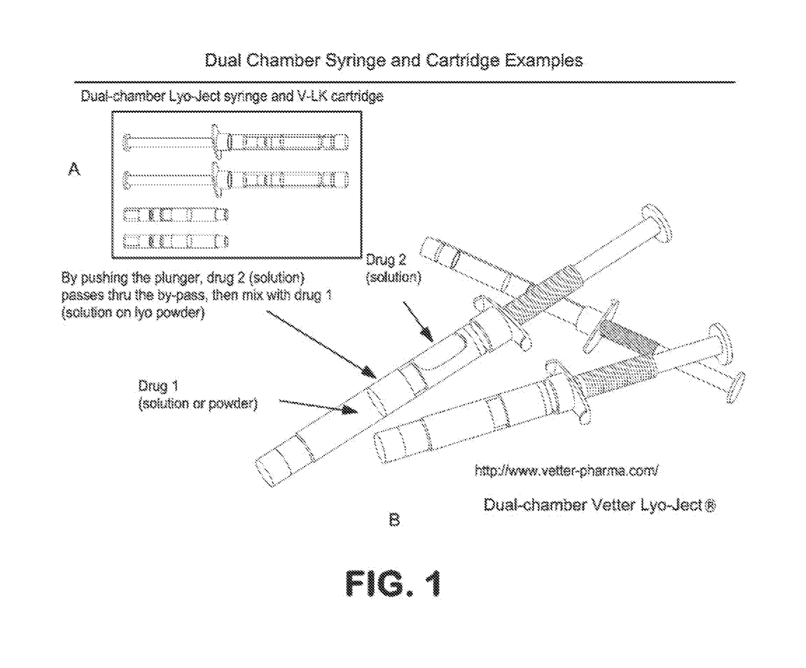

Any compatible device can be used to practice this invention, e.g., a pen as illustrated in FIG. 1. In alternative embodiments, an insulin pump having a continuous glucose monitoring capacity is used, e.g., a MINIMED PARADIGM® REAL-TIME REVEL™ SYSTEM™ (Medtronic), or a device as described in U.S. Pat. Nos. 6,551,276; 6,554,798; 6872200; 6936029; 6979326; 6997920; 7025743; 7109878; and 7819843.

Artificial Pancreas

In alternative embodiments, the invention provides an artificial pancreas comprising a formulation of the invention having an optimized P:I ratio, or that is capable of delivering an optimized P:I ratio of the invention, or is capable of mixing and delivering an optimized P:I ratio of the invention. In one embodiment, an artificial pancreas of the invention comprises an insulin pump under closed loop control using real-time data from a continuous blood glucose sensor, and optionally also can comprise an on-board computer or computer program product, as discussed below.

Automated and Computer Systems and Computer Implemented Methods

The desired ratio of pramlintide:insulin can be determined manually by the subject or automatically, e.g., by use of device of the invention (e.g., an insulin pump or an artificial pancreas) that is manufactured and configured to automatically mix and administer a desired P:I ratio formulation, e.g., via a device of the invention comprising a computer and a computer-implemented method of the invention able to mix or determine and mix a desired P:I ratio formulation, optionally taking into consideration basal insulin levels. In alternative embodiments, the desired ratio of pramlintide:insulin is injected or administered by bolus injection or administration of preset amounts of insulin and pramlintide in separate pens, syringes, containers, compartments and the like. In yet a further embodiment, a device of the invention is configured or manufactured to administer a desired P:I ratio formulation intermittently an amount sufficient to maintain a basal level of insulin activity throughout a day, and the pramlintide:insulin can be administered at a therapeutically effective amount as a bolus prior to a meal.

In alternative embodiments, the methods of the invention, in whole or in part, require implementation using a device, machine, computer system or equivalent, within which a set of instructions for causing the computer or machine to perform any one or more of the protocols or methodologies of the invention may be executed, e.g., mixing and/or administering a desired P:I ratio formulation.

In alternative embodiments, the machine may be connected (e.g., networked) to other machines, e.g., in a Local Area Network (LAN), an intranet, an extranet, or the Internet, or any equivalents thereof. The machine may operate in the capacity of a server or a client machine in a client-server network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. The term “machine” shall also be taken to include any collection of machines, computers or products of manufacture that individually or jointly execute a set (or multiple sets) of instructions to perform any one or more of the methodologies of the invention. For example, in alternative embodiments, a device of the invention measures blood sugar (e.g., using continuous glucose monitoring (CGM) or equivalent to measure glucose values continuously over time through the sensor inserted under the skin), and relays this information to a “onboard” computer or to a remote computing device capable of implementing a method of the invention, e.g., calculate the amount of pramlintide and insulin “premix” formulations to administer separately, or to mix and administer, to administer a final desired amount of P:I ratio to the patient.

In alternative embodiments, an exemplary computer system of the invention comprises a processing device (processor), a main memory (e.g., read-only memory (ROM), flash memory, dynamic random access memory (DRAM) such as synchronous DRAM (SDRAM) or Rambus DRAM (RDRAM), etc.), a static memory (e.g., flash memory, static random access memory (SRAM), etc.), and a data storage device, which communicate with each other via a bus.

In alternative embodiments, a processor represents one or more general-purpose processing devices such as a microprocessor, central processing unit, or the like. More particularly, the processor may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processor may also be one or more special-purpose processing devices such as an application specific integrated circuit (ASIC), a field programmable gate array (FPGA), a digital signal processor (DSP), network processor, or the like. In alternative embodiments the processor is configured to execute the instructions (e.g., processing logic) for performing the operations and steps discussed herein.

In alternative embodiments the computer system further comprises a network interface device. The computer system also may include a video display unit (e.g., a liquid crystal display (LCD) or a cathode ray tube (CRT)), an alphanumeric input device (e.g., a keyboard), a cursor control device (e.g., a mouse), and a signal generation device (e.g., a speaker).

In alternative embodiments, the data storage device (e.g., drive unit) comprises a computer-readable storage medium on which is stored one or more sets of instructions (e.g., software) embodying any one or more of the protocols, methodologies or functions of this invention. The instructions may also reside, completely or at least partially, within the main memory and/or within the processor during execution thereof by the computer system, the main memory and the processor also constituting machine-accessible storage media. The instructions may further be transmitted or received over a network via the network interface device. The computer-readable storage medium and data storage device are built within a device of the invention, or alternatively, are remotely located but capable of instructing the device, e.g., to mix and administer, or to administer separately, the correct amount of pramlintide and insulin such that the desired P:I ratio is administered to the patient.

In alternative embodiments the computer-readable storage medium is used to store data structure sets that define user identifying states and user preferences that define user profiles. Data structure sets and user profiles may also be stored in other sections of computer system, such as static memory.

In alternative embodiments, while the computer-readable storage medium in an exemplary embodiment is a single medium, the term “machine-accessible storage medium” can be taken to include a single medium or multiple media (e.g., a centralized or distributed database, and/or associated caches and servers) that store the one or more sets of instructions. In alternative embodiments the term “machine-accessible storage medium” can also be taken to include any medium that is capable of storing, encoding or carrying a set of instructions for execution by the machine and that cause the machine to perform any one or more of the methodologies of the present invention. In alternative embodiments the term “machine-accessible storage medium” shall accordingly be taken to include, but not be limited to, solid-state memories, and optical and magnetic media.

In alternative embodiments, information and signals are represented using any technology and/or technique known in the art. For example, data, instructions, commands, information, signals, bits, symbols, and chips used to practice the compositions (devices, computers) and methods of the invention can be represented by voltages, currents, electromagnetic waves, magnetic fields or particles, optical fields or particles, or any combination thereof.

### Exemplary Formulations of the Invention

In alternative embodiments, the invention provides compositions, devices, formulations, kits and the like comprising: (i) a pramlintide or a pramlintide peptide, or a physiologically acceptable salt thereof; and (ii) a human insulin or a human insulin peptide (HIP) or an analog thereof, or a physiologically acceptable salt thereof. In alternative embodiments of the compositions, devices, formulations, kits and the like:
the pramlintide or pramlintide peptide is in a liquid formulation and the human insulin or human insulin peptide (HIP) is formulated in a dried formulation, such that when the pramlintide or pramlintide peptide and insulin or insulin peptide are mixed the mixture comprises a liquid formulation of the invention; or,
the human insulin or human insulin peptide (HIP) is in a liquid formulation and the pramlintide or pramlintide peptide is formulated in a dried formulation, such that when the pramlintide or pramlintide peptide and human insulin or human insulin peptide (HIP) are mixed the mixture comprises a liquid formulation of the invention, or
the insulin or insulin peptide and the pramlintide or pramlintide peptide are both formulated in a dried formulation, such that when the pramlintide or pramlintide peptide and human insulin or human insulin peptide (HIP) are mixed the mixture comprises a liquid formulation of the invention.

In alternative embodiments, the liquid for reconstituting the dried formulation or formulations are contained in or stored in or within the device or product of manufacture, or, the device or product of manufacture is configured or manufactured to receive input of a liquid to reconstitute the dried formulation.

In alternative embodiments of the compositions, devices, formulations, kits and the like, the pharmaceutical composition or a formulation comprises a:

=>Liquid Co-Formulation.

In one embodiment, the final co-formulation is made by mixing of the individual two components by a patient, health practitioner, pharmacist, or a product of manufacture or a device, e.g., a pump, injector, pen and the like. In one embodiment, the final co-formulation has at least 1 day to about 1 month in-use stability. Any U/ml insulin concentration can be in a final formulation, but commonly used embodiments are 100 U/ml and 500 U/ml insulin, or between about 100 U/ml and 500 U/ml insulin.

However, where a commercially available, regulatory agency-approved, e.g. FDA or EMA, 100 U/ml human insulin or HIP product is used as one of the two individual liquid components prior to mixing to generate the liquid formulation of the invention, the U/ml insulin concentration of the final-co-formulation will be less than 100 U/ml, preferably from 20 to 90 U/ml, more preferably from 20 to 80 U/ml, even more preferably from 25 to 75 U/ml, and most preferably from 25 to 60 U/ml. Such approved 100 U/ml human insulin or HIP products include HUMALIN R™, NOVOLIN R™, HUMALOG™, NOVOLOG™, APIDRA™ and their equivalent generic or biosimilar drug products, i.e. that have an “AB” rating or are interchangeable with the reference drug product.

In one embodiment, a modified liquid pramlintide formulation is used as the liquid component prior to mixing, e.g., a modified SYMLIN™ is used (the commercially available SYMLIN™ has 0.6 mg/ml or 1.0 mg/ml pramlintide). In the modified liquid pramlintide formulation, the acetate buffer is modified such that after mixing of liquid pramlintide and insulin, the final acetate buffer concentration (or equivalent concentration for another buffer) is between about 15 to 100 mM, with the final buffer concentration being the equivalent of 15 mM to 100 mM acetate, and a common embodiment having between 17 to 80 mM acetate or equivalent, more alternatively at about 20 to 60 mM acetate or equivalent final buffer concentration. As noted herein, by equivalent concentration of other buffer is meant a concentration that provides the same buffer capacity as the stated concentration of acetate buffer at the stated pH of the final formulation as described herein, e.g. about pH 4.0 In alternative embodiments, the final formulation osmolarity is held at between about 250 to about 400 milliosmoles, with a common embodiment being at about 290 milliosmoles; optionally this can be achieved by using about 3.5% to 5% mannitol, or equivalent.

=>Dried Pramlintide Composition Reconstituted with Liquid Insulin

In one embodiment, a commercially available, regulatory-agency-approved human insulin or HIP drug product is used, e.g., insulin formulated as 100 U/ml Insulin or 500 U/ml, e.g., include HUMALIN R™, NOVOLIN R™, HUMALOG™, NOVOLOG™, APIDRA™ and their equivalent generic or biosimilar drug products, i.e. that have an “AB” rating or are interchangeable with the reference drug product. Alternative embodiments, can provide a final formulation of 100 U/ml of 500 U/ml insulin, since addition of pramlintide powder will not change volume of liquid.

In alternative embodiments, pramlintide powder comprises pramlintide peptide, buffer for desired pH and pH stability capacity (i.e. buffer capacity) in the final formulation, and a bulking agent to improve rapid dissolution, act as a cryoprotectant and/or contribute to final isotonicity, which optionally can be between 250 to 400 milliosmole (mOsm/kg). The buffer can be non-volatile and/or non-chelating, for example, a glutamic acid or glutamate buffer is used. The concentration of a glutamate buffer at the desired pH herein can be that amount that provides a buffer capacity equivalent to that of 15 to 100 mM acetate buffer, or about 8 mM to about 50 mM.

=>Dried Insulin Reconstituted with a Liquid Pramlintide Peptide or Commercially Available SYMLIN™.

In alternative embodiments, the SYMLIN™ is used at the commercially available 0.6 mg/ml or 1.0 mg/ml pramlintide formulations. In alternative embodiments, this reconstitution can provide a final formulation of about 100 U/ml or 500 U/ml, or between about 100 U/ml or 500 U/ml, since the insulin powder will not change volume of the pramlintide liquid.

In alternative embodiments, insulin powder will need human insulin or HIP peptide, sufficient zinc to provide human insulin hexamer or the desired form of the HIP, a bulking agent (for example, a polyol, a mannitol, and the like) to improve rapid dissolution and/or act as a cryoprotectant and/or contribute to final isotonicity, which optionally can be between about 250 to 400 mOsm. In one embodiment, the insulin solution is pH titrated to neutral (about pH 7) prior to lyophilizing to enable a soluble insulin before drying. In one embodiment, no glycerin is added.

In alternative embodiments, if SYMLIN® or an equivalent formulation is used, the final formulation will comprise the ingredients of SYMLIN®, which comprises an acetate salt form of pramlintide formulated as a clear, isotonic, sterile solution for subcutaneous (SC) administration, a metacresol as a preservative, D-mannitol as a tonicity modifier, and acetic acid and sodium acetate as pH modifiers. In alternative embodiments pramlintide acetate formulated at 1000 mcg/mL (ug/mL) or 600 mcg/mL is used; a disposable multidose SYMLINPEN® pen-injector contains 1000 mcg/mL of pramlintide (as acetate); and SYMLIN® vials contain 600 mcg/mL of pramlintide (as acetate). In alternative embodiments, the 1000 mcg/mL or 600 mcg/mL is mixed to get intermediary concentrations (e.g., between about 1000 mcg/mL and 600 mcg/mL).

=>Dried, Co-Formulated Pramlintide and Insulin Reconstituted with a Diluent.

In alternative embodiments, pramlintide peptide is mixed with insulin peptide, bulking agent, zinc at a minimum. In one embodiment, when reconstituted, the dried co-formulation is mixed or dissolved with a diluent comprising a water, a buffer and a preservative, and optionally an isotonic agent as needed.

In alternative embodiments, a dried pramlintide peptide is mixed with dried insulin peptide, bulking agent, zinc and a buffer, which optionally is non-volatile, such as a glutamate buffer. In one embodiment, when reconstituted, the dried co-formulation is mixed or dissolved with a diluent comprising a water and a preservative, and optionally an isotonicity agent. In one embodiment, the buffer is a non-chelating buffer. In one embodiment, the buffer is a non-volatile buffer, but this is only required if the buffer is in dry powder. Volatile buffers such as acetate can be in liquids or liquid diluents. A preservative also can be present in the diluent, e.g., a metacresol.

=>Liquid Commercial 500 U/Ml Insulin Plus Liquid Commercial SYMLIN® Plus a Diluent.

In one embodiment, mixing these three liquid “starting materials” results in a co-formulation of 100 U/ml insulin, or a lesser other U/ml concentration. In one embodiment, the diluent can have water and as needed: for example, the diluent can comprise a buffer, an isotonicity agent, a zinc, the amounts of which depend on the volume of SYMLIN® mixed with the volume of 500 U/ml insulin, which depends on P:I ratio desired. The final formulation will comprise the ingredients of SYMLIN®, which comprises an acetate salt form of pramlintide formulated as a clear, isotonic, sterile solution for subcutaneous (SC) administration, a metacresol as a preservative, D-mannitol as a tonicity modifier, and acetic acid and sodium acetate as pH modifiers. In alternative embodiments pramlintide acetate formulated at 1000 ug/mL or 600 ug/mL is used.

In one embodiment, a commercially insulin is used, e.g., insulin formulated as 100 U/ml Insulin, e.g., an aspart, a NOVOLOG™ or NOVOLIN R™ or a NOVORAPID™ (Novo Nordisk, Bagsværd, Denmark); a glulisine or an APIDRA™ (Sanofi S.A., Paris, France); a lispro, an insulin lispro protamine or a HUMALOG™ (Eli Lilly and Company, Indianapolis, Ind.); a HUMULIN R™, (Eli Lilly and Company, Indianapolis, Ind.). Alternative embodiments, can provide a final formulation of 100 U/ml of 500 U/ml insulin, since addition of pramlintide powder will not change volume of liquid.

In alternative embodiments, the invention provides devices, compositions of matter and the like, and methods for using them, having pramlintide and the insulin in separate liquid formulations. For example, in alternative embodiments, where the pramlintide (pram) and the insulin are in separate liquid formulations, with pram at about pH 4.0, pH 4.1 or pH 4.2, and with insulin either at about pH 7 or at about pH 4.0, pH 4.1 or pH 4.2, where mixture yields a final pH of about pH 4.0, pH 4.1 or pH 4.2:

| | |
|---|---|
| Two Individual | One vial for each drug solution |
| Vials | Prior to dose, draw desired dose of drug |
| | solution into syringe, then mix drug solutions well in |
| | the syringe |
| Three Individual | One vial for SYMLIN, one vial for HUMULIN |
| Vials | R or NOVLIN R, and one vial for buffer that provides |
| | additional buffer capacity and about pH 4.0, pH 4.1 or |
| | pH 4.2 |
| | The added buffer solution avoids insulin |
| | precipitation after mixing |
| | Prior to dose, draw desired dose of drug |
| | solutions into syringe, then mix drug solutions well in |
| | the syringe |
| Dual-chamber | One chamber or cartridge for each drug |
| Syringe or Dual | solution |
| Cartridge; | Push plunger to mix two drug solutions well |
| Autoinjector with | prior to dose |
| Dual-chamber | In-use stability needed for multiple doses |
| Cartridge | |
| Dual-chamber | One chamber for each drug solution |
| Insulin Pump | Mix two drug solutions thru “T” connector of |
| | tubing |
| | To achieve desired dose ratio by adjusting |
| | delivery rate of each chamber |

In alternative embodiments, where the pramlintide is a liquid and the insulin is dried, and mixture is at about pH 4.0, pH 4.1 or pH 4.2:

| | |
|---|---|
| Two Individual Vials | One vial for each drug |
| | Prior to dose, draw desired dose of |
| | drug solution into syringe, then mix drug |
| | solutions well in the syringe. |
| Dual-chamber Syringe or | One chamber for each drug |
| Dual Cartridge; | Push plunger to allow pramlintide |
| Autoinjector with Dual- | solution to mix with insulin lyo powder to |
| chamber Cartridge | form a homogeneous solution prior to dose |
| | In-use stability needed for multiple |
| | doses |
| Dual-chamber Insulin | One chamber for each drug |
| Pump | Push plunger to allow pramlintide |
| | solution to mix with insulin lyo powder to |
| | form a homogeneous solution prior to dose |

In alternative embodiments, the pramlintide acetate solution is formulated at pH 4; a higher buffer capacity may be needed to avoid insulin precipitation after mixing; Insulin formulated can be as a lyophilized (dried) powder (pH 7).

In alternative embodiments, where the pramlintide and Insulin are co-formulated as dried powder for reconstitution to a pH at about pH 4.0, pH 4.1 or pH 4.2:

| | |
|---|---|
| Two Vials | One vial contains pramlintide |
| | acetate and insulin co-lyo powders |
| | One vial contains pH 4 diluent |
| | solution |
| | Reconstitute co-lyo powders with |
| | diluent solution to form a homogeneous |
| | solution prior to dose |
| | In-use stability needed for |
| | multiple doses |
| Dual-chamber Vial, Pen | One chamber for diluent solution, |
| Syringe or Cartridge; | one chamber for co-lyo powders |
| Autoinjector with Dual- | Fix pramlintide/insulin dose ratio |
| chamber Syringe, Cartridge or | Push plunger to reconstitute co- |
| Pen | lyo powders to form a homogeneous |
| | solution prior to dose |
| | In-use stability needed for |
| | multiple doses |
| Dual-chamber Insulin Pump | One chamber for diluent solution, |
| | one chamber for co-lyo powders |
| | Fix pramlintide/insulin dose ratio |
| | Push plunger to reconstitute co- |
| | lyo powders to form a homogeneous |
| | solution prior to dose |
| | Need to evaluated drug solution |
| | compatibility & stability with tubing |

In an alternative embodiment, the invention also provides a dual chamber pen with dual barrels: 2 chambers, one filled with pramlintide, the other with insulin. Push the plunger and pramlintide and insulin come out of separate needles, that are attached (e.g., like some of the epoxy glues). Insulin and pramlintide are only mixed once they come out of the needle tip. In an alternative embodiment, the invention also provides a dual chamber pen as illustrated in FIG. 1.

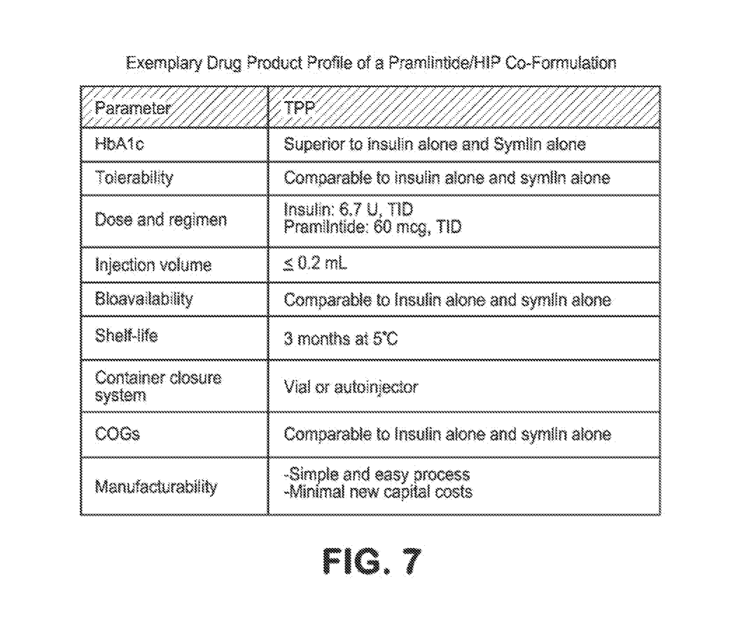

FIG. 7 described an exemplary formulation of the invention comprising Insulin: 6.7 U, TID and Pramlintide: 60 mcg, TID. As with any formulation of the invention, this exemplary formulation can be stored and delivered using a Vial with: Bolus S.C. injection; Pump infusion; Cartridge in an autoinjector, or Bolus S.C. injection.

Exemplary formulations of the invention also can have a pH of between about 3.0 and 5.5, about 3.5 to 4.5, about 3.7 to about 4.3, about 4.0, or a pH of about 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4 or 5.5; for example:

Formulation at pH 4
2 individual liquid formulations, mix before use and use for 1 month
pramlintide in one vial with higher buffer capacity than SYMLIN ™
Insulin in another vial and used “as-it-is” from one commercial insulin
product (e.g. HUMULIN R ™ or NOVOLIN R ™)
Lyophilized insulin + pramlintide as diluent
Lyophilized pramlintide + insulin as diluent
Lyophilized co-formulation

In alternative embodiments, the pH range is more than 3.5 to less than 4.4, and more typically 3.8 to less than 4.4. Alternative pramlintide peptide formulations and final co-formulation pH include about 3.8, 3.85, 3.9, 3.95, 4.0, 4.05, 4.1, 4.15, 4.2, 4.25, 4.3, and 4.35. In alternative embodiments, a pH range is each range that is selected from the group of ranges where the pH values 3.8, 3.85, 3.9, 3.95, 4.0, 4.05, 4.1, 4.15, 4.2, 4.25, 4.3, and 4.35 are selected as a lower and an upper end of the range, including for example 3.8 to 4.35, 3.85 to 4.35, 3.85 to 4.2, 3.85 to 4.15, 3.8 to 4.2, 3.9 to 4.1, 3.9 to 4.0, and 4.0 to 4.1. In alternative embodiments, the pH range is about pH 3.9, 4.0 and 4.1, and ranges 3.9 to 4.1, 3.9 to 4.0, and 4.0 to 4.1. It has been found that less acidic pH, generally 3.8 and above, will minimize acid catalyzed deamidation of insulin and/or pramlintide.

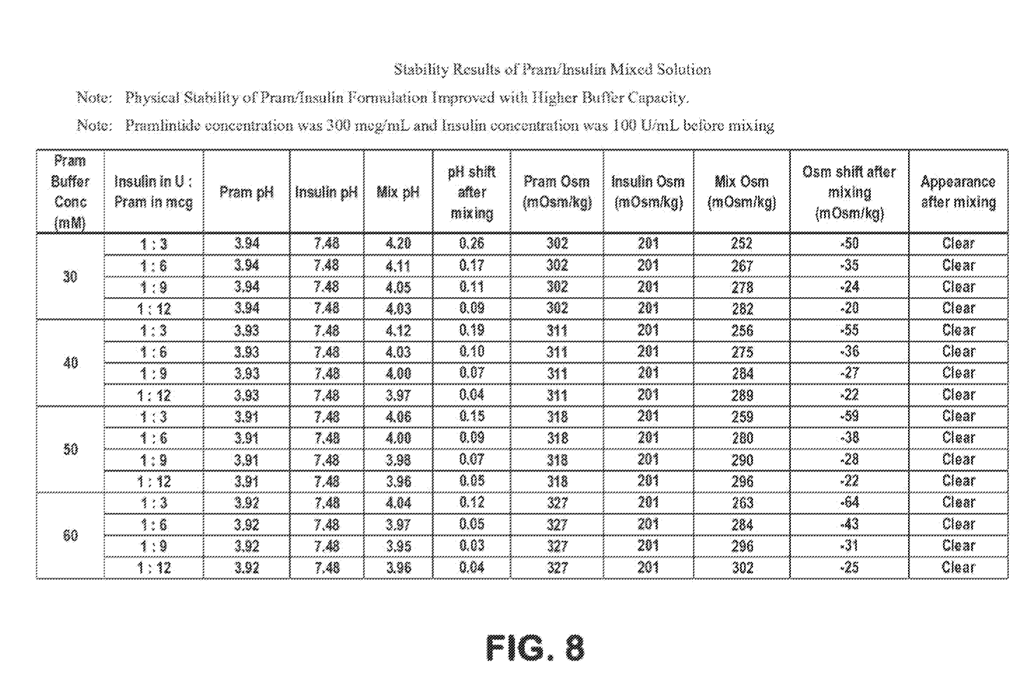

FIG. 8 describes stability results of exemplary pramlintide:insulin mixed solutions of the invention, and data showing that the physical stability of the exemplary pramlintide:insulin formulations improved with higher buffer capacity. The first column lists acetate buffer concentration in the pramlintide premix solution (the premix insulin solution is in pH-adjusted but not buffered water, as are most commercially available insulins used to practice the invention); the second column lists the final pramlintide:insulin ratio after mixing; the third and fourth columns list the pH's of the respective “premix” solutions, with the fifth column listing the final pH of the mixed co-formulation (the sixth column calculates and lists the pH shift after mixing the “premix” solutions); the seventh and eighth columns list the osmolarities of the respective “premix” solutions, with the ninth column listing the final osmolarity after mixing (the tenth column lists the osmolarity shift from premix to postmix solutions); and the eleventh or last column describes that all the solutions are “clear”, i.e., indicating that the pramlintide and the insulin are dissolved in solution. The samples of FIG. 8 were also examined for particle size using hydrodynamic light scattering (DLS). At t=0 all samples displayed acceptable Z-average diameter particles compared to placebo and to SYMLIN. In addition, visually all samples scored as less than 10 NTU, the lowest reference standard used, under Tyndall light. In FIG. 8, “U” means international unit; for example in the first row 1:3 means 1 U, or one international unit, of insulin: to 3 μgm, or 3 mcgm, pramlintide.

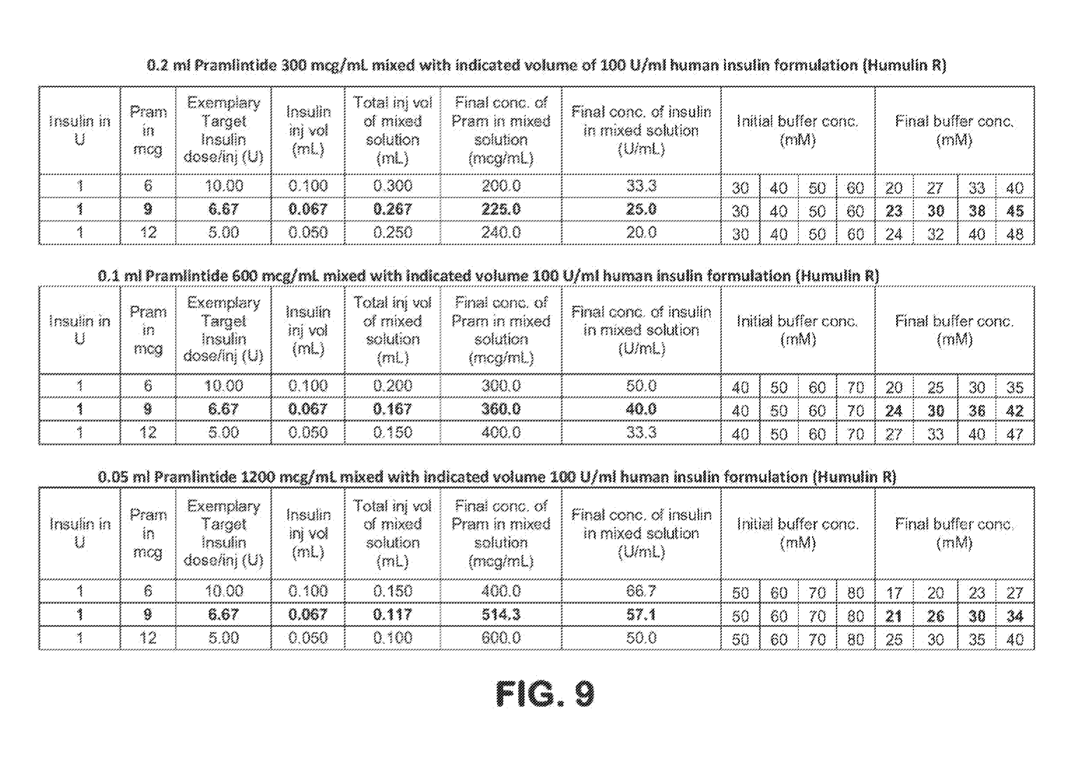

FIG. 9 also describes exemplary pramlintide:insulin co-formulations of the invention, using pramlintide at 300, 600 and 1200 mcg/mL (μg/mL) as “starting” or “premix” solutions. Type 1 diabetes patients use 10-15 U of insulin per meal, which is equivalent to 0.1 to 0.15 mL of 100 U/mL product. Note that in the alternative exemplary formulations of FIG. 9, the insulin concentrations of all the “starting” solutions are 100 U per ml, which is a common formulation for commercially available insulins that can be used to practice this invention. The final volume of the post-mix co-formulation solution, the amount of pramlintide and insulin actually injected (the amount in the post-mix co-formulation solution), and the final concentrations of pramlintide and insulin in the post-mix co-formulation solution, are listed. Shown are the initial acetate buffer concentrations in the pramlintide premix solutions (the initial, or “premix” insulin is formulated in water, which is typical for commercially available insulins which can be used to practice this invention), and the “final” buffer concentration (all diluted as compared to the premix concentration as the insulin is in water). In typical embodiments, a lower limit for the final “mixed” co-formulation solution is 17 mM acetate, or in alternative embodiments using other buffers, the equivalent of a 17 mM acetate buffer.

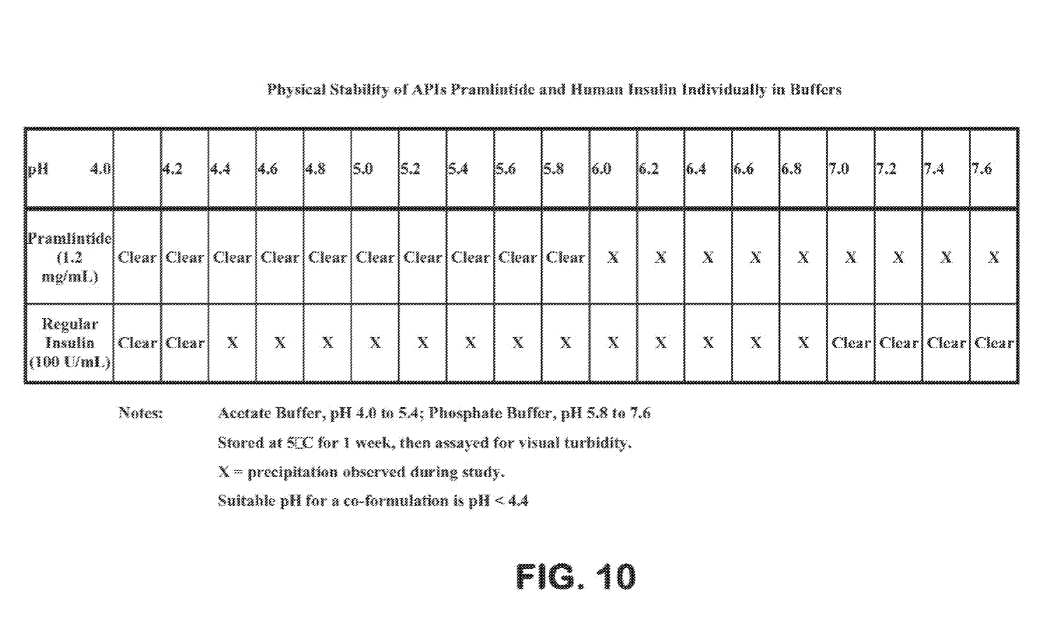

FIG. 10 depicts solubility versus pH for pramlintide and human insulin, individually formulated. Samples were prepared at the pH indicated; for insulin in HUMULIN R formulation adjusted to the indicated pH; for pramlintide in SMYLIN formulation adjusted to the indicated pH. Samples were stored at 5 degrees Centigrade and observed for visual turbidity each day. Samples marked “clear” were clear throughout the study. Samples marked “X” displayed precipitation during the study. This study supports the aspect that pramlintide peptide and human insulin or HIP can be co-formulated or can be mixed (e.g. mixed and injected as a bolus and/or a basal injection by an “insulin” pump) preferably at a pH less than pH 4.4, and will be a suitable for immediate use or for multi-day in-use period, e.g. up to 1 month.

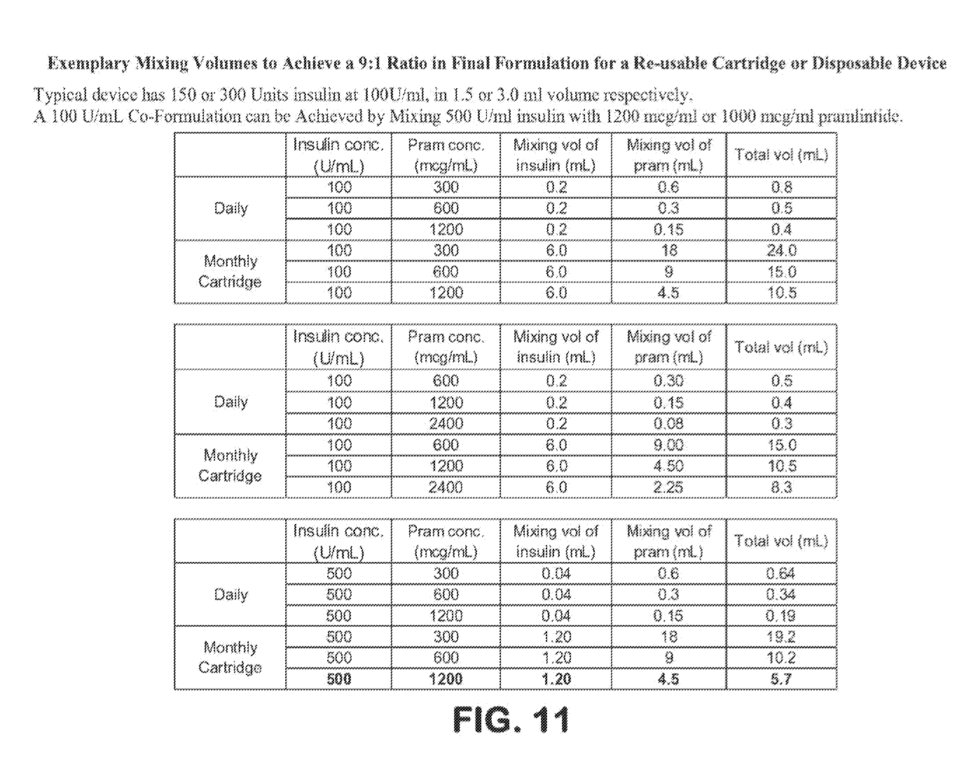

FIG. 11 describes alternative exemplary pramlintide/insulin formulations, including the insulin and the pramlintide “premix” solutions (first two columns), the mixing volume in mL of the respective “premix” pramlintide and insulin formulations, and the total volume of liquid in each cartridge; note that for all exemplary formulations the formulations are used. Alternative daily volume and monthly cartridges are listed (exemplary formulations include daily, weekly and monthly dosage units, e.g., in cartridges, pens, vials and the like). In alternative embodiments, a device of the invention comprises 150 or 300 units insulin at 100 U/ml, in 1.5 or 3.0 ml volume, respectively; and in one embodiment, a 100 U/ml co-formulation can be achieved by mixing 500 U/ml insulin with 1200 mcg/ml or 1000 mcg/ml of pramlintide.

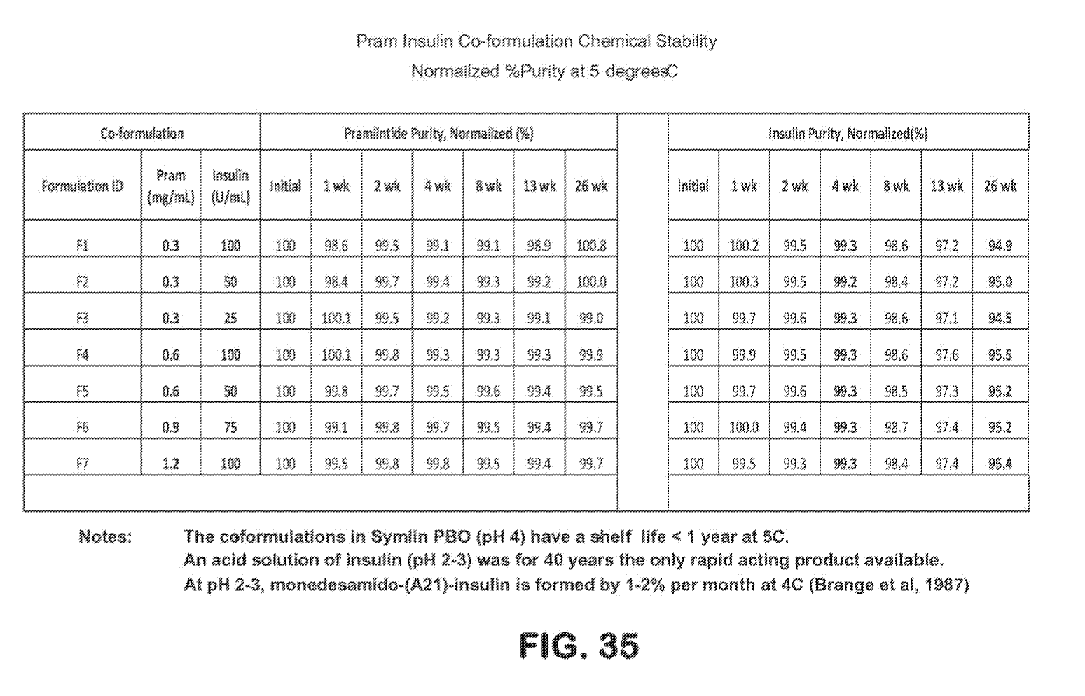

FIG. 35 depicts the stability of pramlintide and of human insulin co-formulated in SYMLIN™ placebo buffer, at pH 4.0, with no additional buffering capacity, and stored at 5 degrees Centigrade. Purity was determined via RP-HPLC and is normalized to time=0 purity. The co-formulations in SYMLIN placebo (PBO) (pH 4) have a shelf-life <1 year at 5 degrees Centigrade. However, at 25 degrees Centigrade, unacceptable degradation of the insulin occurred during the first week as shown in the subsequent figure. Note that an acid solution of insulin (pH 2-3) was for 40 years the only rapid acting product available, even though at pH 2-3, monodesamido-(A21)-insulin is formed by 1-2% per month at 4C (Brange et al, 1987).

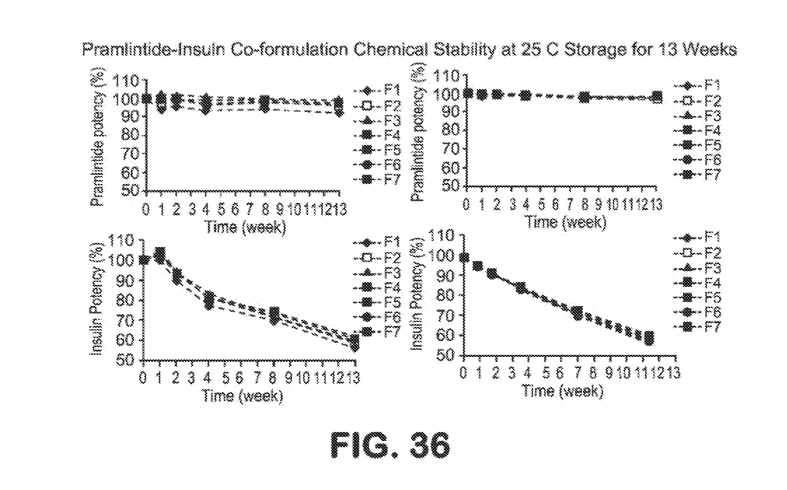

FIG. 36 depicts the stability of pramlintide and of human insulin co-formulated in SYMLIN™ placebo buffer, at pH 4.0, with no additional buffering capacity, and stored at 25 degrees Centigrade. Although pramlintide is stable over the 13 week study, human insulin rapidly degrades. At 25 degrees C, insulin in co-formulations showed significant potency and purity (40%) loss during storage. It has been discovered herein that the mixing of unmodified SYMLIN with commercially available human insulin drug product, e.g. HUMULIN R, results in an increase in pH (data not shown) that leads to the observed insulin instability. The inventors found that the stability of human insulin can be achieved by providing sufficient buffer capacity to maintain the target acidic pH, e.g. pH 4.0. Accordingly, in one aspect this can be achieved by modifying the buffer capacity of the SYMLIN formulation as described herein.

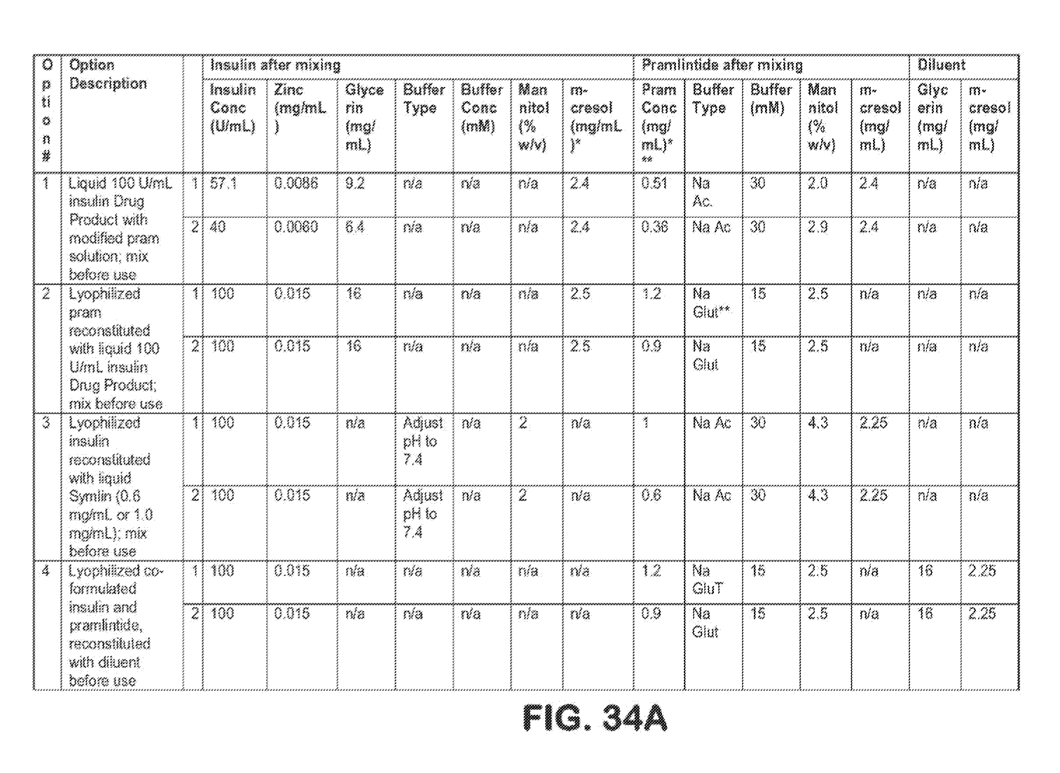

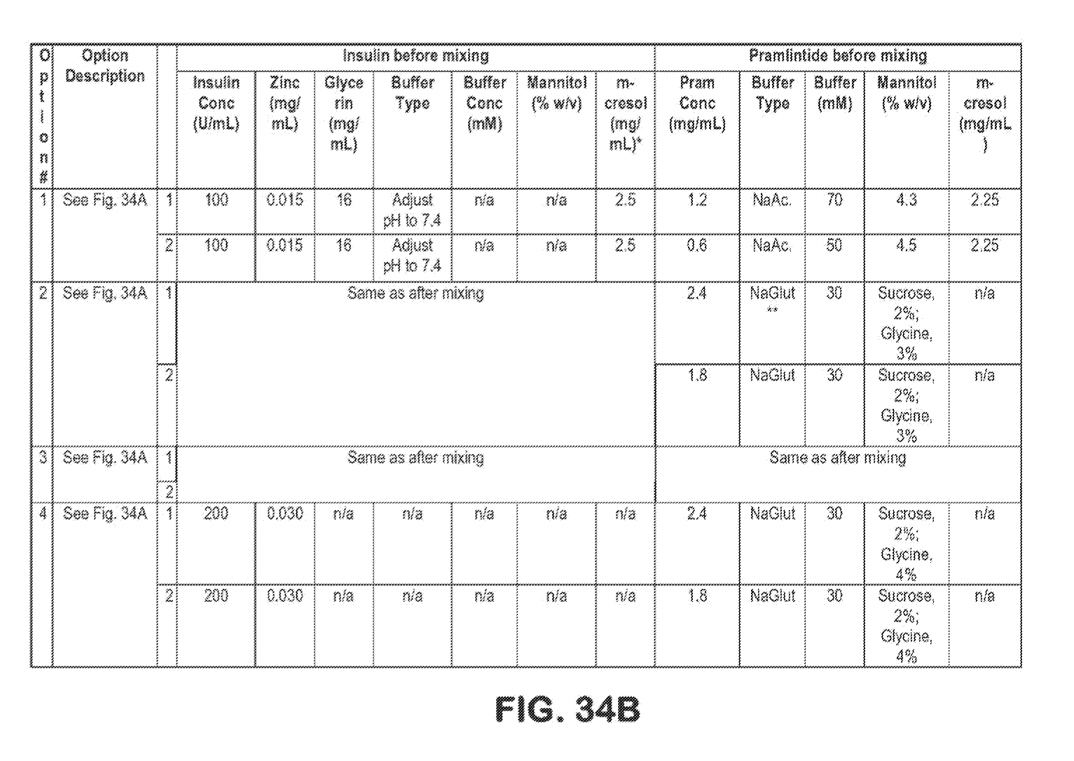

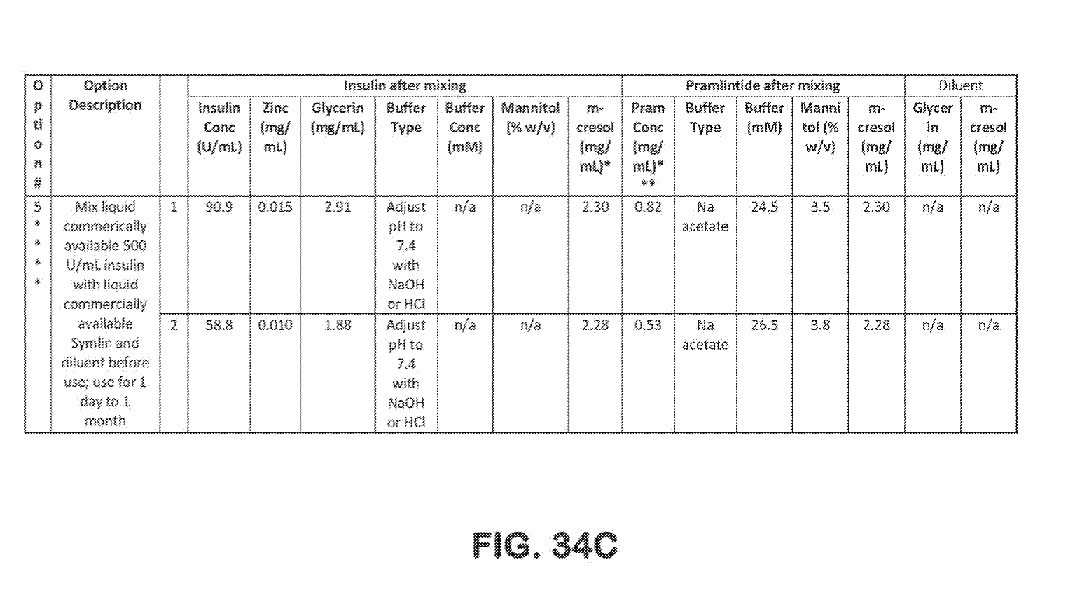

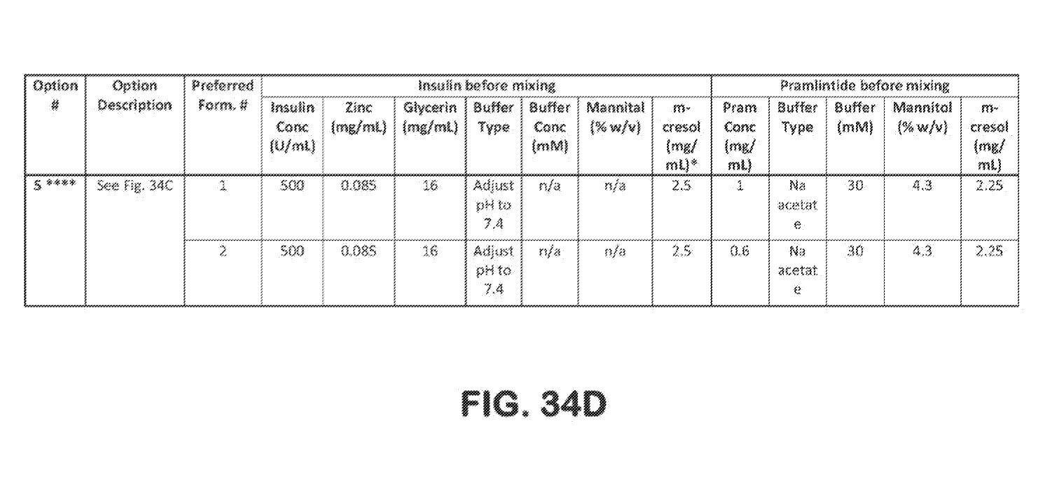

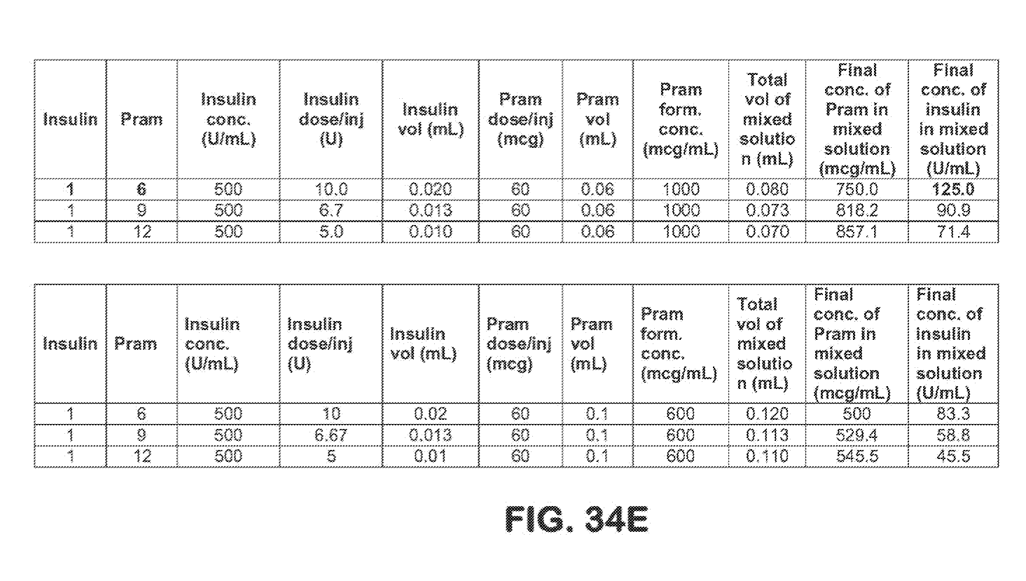

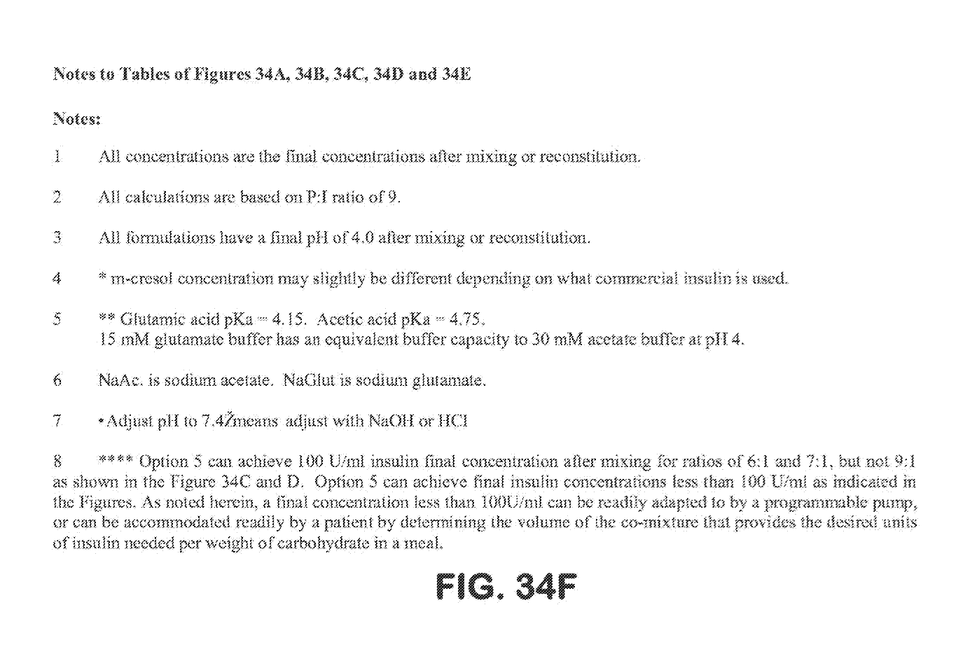

FIGS. 34A, 34B, 34C, 34D and 34E, also describe alternative exemplary pramlintide/insulin formulations, listed as “options” or embodiments 1 to 5. The insulin and the pramlintide “premix” solutions are described in the “option description” column.

For example, embodiment (or “option”) 1, comprises use of a “premix” solution comprising a “modified” pramlintide solution and a 100 U/ml insulin “drug product” solution, i.e., an insulin solution from a commercially available 100 U/ml insulin source (all commercially available insulins are in a water solution having only a preservative m-cresol, where the m-cresol concentration might differ depending on the commercial insulin source).

Embodiment (or “option”) 2, comprises use of a dried, or lyophilized pramlintide reconstituted with a commercially available 100 U/ml insulin source.

Embodiment (or “option”) 3, comprises use of a dried, or lyophilized insulin reconstituted with either a 0.6 mg/mL or a 1.0 mg/mL SYMLIN® (which is commercially available at either 1000 ug/mL or 600 ug/mL in a disposable multidose SYMLINPEN® pen-injector containing 1000 g/mL of pramlintide or 600 g/mL of pramlintide (both as acetate; and both formulations comprise: 2.25 mg/mL of metacresol as a preservative, D-mannitol as a tonicity modifier (at 4.3% (wt/vol), and acetic acid and sodium acetate as pH modifiers, 30 mM acetate at pH of approximately 4.0).

For options (embodiments) 1 to 4 (FIG. 34A, 34B): all listed concentrations are final (to be administered) concentrations of ingredients after mixing of liquids or reconstitution of dried formulation (powder); all four exemplary formulations have a final (to be administered) pramlintide:insulin (P:I) ratio of 9:1; all four exemplary formulations have a final (to be administered) pH of 4.0. See FIG. 34F for further notes to Table 34A; and, for comment (note) 1, in FIG. 34F, all concentrations are the final concentrations after mixing or reconstitution unless otherwise indicated, e.g. in the “before mixing” column.

Option (or embodiment) 5 is a mix of diluent, commercially available 500 U/ml insulin and liquid commercially available SYMLIN®; this exemplary formulation can achieve 100 U/ml insulin final concentration after mixing for ratios of 6:1 and 7:1, but not 9:1 as shown in the FIGS. 34C and D. Option 5 can achieve final insulin concentrations less than 100 U/ml as indicated in the Figures. As noted herein, a final concentration less than 100 U/ml can be readily adapted to by a programmable pump, or can be accommodated readily by a patient by determining the volume of the co-mixture that provides the desired units of insulin needed per weight of carbohydrate in a meal.

For all embodiments, the formulations can be used up to 1 month after mixing, in addition to immediately after mixing, of course.

FIG. 34E illustrates exemplary co-formulations of the invention using varying amounts of commercially available 500 U/mL insulin and liquid pramlintide at 1000 mcg/mL and 600 mcg/mL, both of these two concentrations are commercially available in alternative commercially available SYMLIN® products.

In the exemplary co-formulations of FIG. 34E, note that the final concentration of insulin (U/mL) in a co-formulation (a mixed solution) using a 1000 mcg/mL pramlintide “starting” premix solution is 125.0 (a 6:1 P:I ratio), 90.9 (a 9:1 P:I ratio) and 71.4 U/mL (a 12:1 P:I ratio); and 83.3 (a 6:1 P:I ratio), 58.8 (a 9:1 P:I ratio) and 45.5 (a 12:1 P:I ratio) using a 600 mcg/mL pramlintide “starting” premix solution. Note that the total volume of the final, mixed co-formulation solution also changes.

### Dried or Lyophilyzed Pramlintide and Insulin

In alternative embodiments, the invention provides compositions (e.g., products of manufacture, devices, and the like) comprising and methods using reconstitutable dried pharmaceutical compositions or formulations comprising pramlintide, insulin and/or both pramlintide and insulin, or formulations comprising optimal mixes of pramlintide and insulin at optimized P:I ratios of the invention made using reconstituted dried (e.g., lyophilized) pharmaceutical compositions or formulations comprising pramlintide, insulin and/or both pramlintide and insulin.

The dried pramlintide and insulin can be prepared by spray drying, rotary evaporation, freeze-drying or lyophilization, or any other equivalent methodology.

### Exemplary Protocols for Making Dried Pramlintide and Insulin are:

| Exemplary | | | | | |
|---|---|---|---|---|---|
| Lyophilization | | | | | |
| protocols for | | | | | |
| pramlintide, | | | | | |
| insulin, or | | Ramp | | | |
| pram + | Step | rate | Temp, | Pressure | Duration |
| insulin: Step | description | (C/hr) | C. | (mTor) | (hr) |
| 1 | Loading | | 5 | | 0.5 |
| 2 | Freezing | −15 | −40 | | 3.0 |
| 3 | Freezing | hold | −40 | | 2.0 |
| 4 | Freezing | 10 | −15 | | 2.5 |
| 5 | Freezing | hold | −15 | | 2.0 |
| 6 | Primary drying | hold | −15 | 80 | 1.0 |
| 7 | Primary drying | hold | −15 | 80 | 48.0 |
| 8 | Secondary drying | 10 | 30 | 80 | 4.0 |
| 9 | Secondary drying | hold | 30 | 80 | 12.0 |

### Kits and Instructions

The invention provides kits comprising devices, products of manufacture or compositions for practicing the methods of the invention, including instructions for use thereof. In alternative embodiments, the kits further comprise instructions for practicing a method of the invention.

For example, in one embodiment, the kit comprises premix solutions of pramlintide and insulin for mixing to make an optimized P:I ratio co-formulation of the invention, or for administering the appropriate amount of pramlintide and insulin, albeit separately, to achieve administration of an optimized P:I ratio co-formulation of the invention. The kit can comprise a device or product of manufacture of the invention, e.g., a continuous subcutaneous insulin infusion therapy device; an insulin pump device; an ampoule; a vial; a cartridge; a syringe, cartridge or disposable pen or jet injector; a needleless injector or a needle free injector; a prefilled pen or syringe or cartridge, or a disposable syringe or pen or jet injector; an AUTOPEN™; a two chambered syringe, cartridge or disposable pen or jet injector; a multi-chambered syringe, cartridge or disposable pen or jet injector, or an artificial pancreas.

The invention will be further described with reference to the following examples; however, it is to be understood that the invention is not limited to such examples.

### Example 1

### In Silico Modeling Determines the Efficacy of Pramlintide:Insulin Ratios of the Invention: A Ratio of 9:1 is Optimal

In alternative embodiments, the invention provides formulations, pharmaceutical compositions, devices and other products of manufacture comprising therapeutically effective mixtures of insulin and pramlintide at specific ratios (pramlintide:insulin, or P-I ratios). The in silico studies, as set forth below, determined the pramlintide:insulin ratio for co-administration in Type 1 diabetes, and concluded that a pramlintide dose should be adjusted in parallel with patients' carbohydrate ratio; average CR reduction of 20% appears warranted. These in silico studies show that a P-I ratio of 9 (i.e., 9:1) is likely to be optimal in terms of pramlintide efficacy and safety (e.g., hypoglycemia).

These in silico studies (experiment 1, below) also show that a P-I ratio of 3 is not better than insulin alone (placebo); it only delays the postprandial blood glucose peak, but does not attenuate its magnitude, and that without adjusting subjects individual CR for pramlintide use, P-I ratios of 6, 8, 9, 10 and 12 resulted in increased hypoglycemia, with 9%, 10%, 11%, 12% and 15% of subjects experiencing glucose levels <50 mg/dl.

These in silico studies (experiment 2, below) also show that a P-I ratio of 3 was no more efficient than placebo with 62% vs. 71% in A-zone; and P-I ratios of 6, 8, 9, 10 and 12 resulted in significant improvement of postprandial glucose control: 81%, 89%, 90%, 89% and 88% of subjects in Control Variability-Grid Analysis (CVGA) A-zone, respectively, and no hypoglycemia. The CVGA displays maximum blood glucose on the y axis with increasing values and the minimum blood glucose on the x-axis with decreasing values. Measurements in the A zone are desirable, because they exemplify low hyperglycemic values without hypoglycemia. On the other spectrum, values in grid E show high hyperglycemic values with many measurements in the hypoglycemic range.

On Jan. 18, 2008, FDA accepted as a substitute for animal trials in certain closed-loop control experiments, a computer simulator of the human metabolic system developed at UVA and the University of Padova, Italy. This set a precedent for fast and cost-effective in silico pre-clinical trials [1]. The preliminary data discussed below using in silico modeling on aggregated data suggests that the integration of pramlintide within a dual-hormone closed-loop control could increase time spent within therapeutic range up to 90% (based on aggregate estimates). It was concluded that the prospect for such integration needs to be explored. Additional evidence supports the feasibility of such an approach.

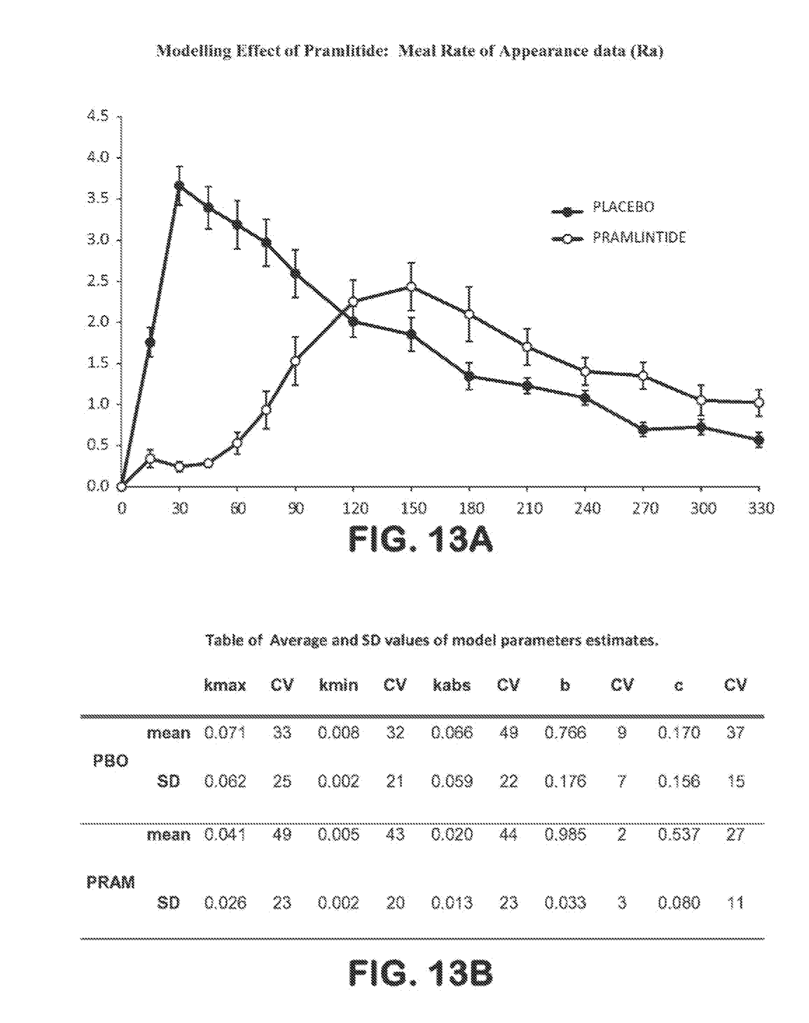

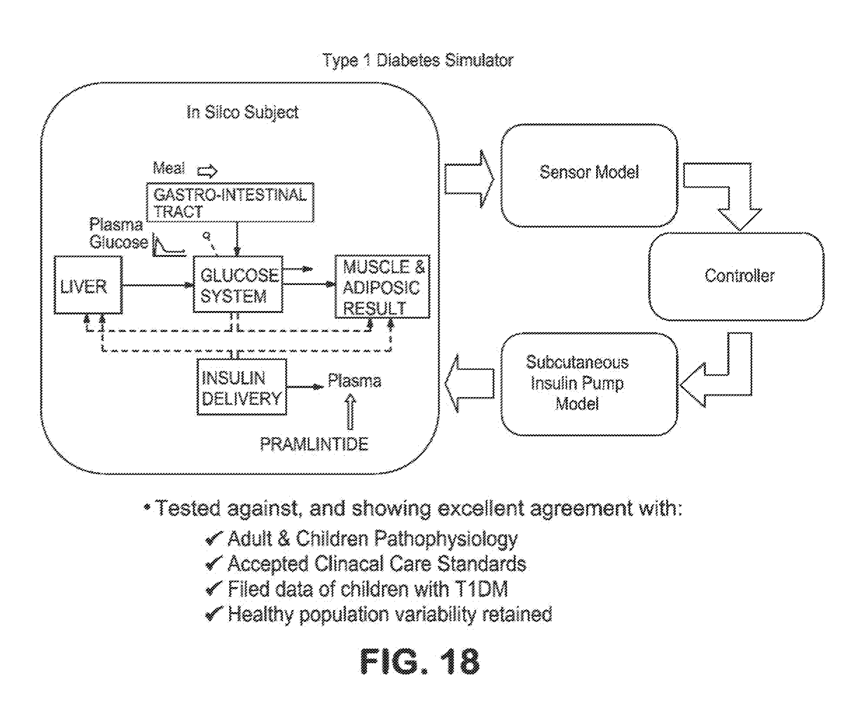

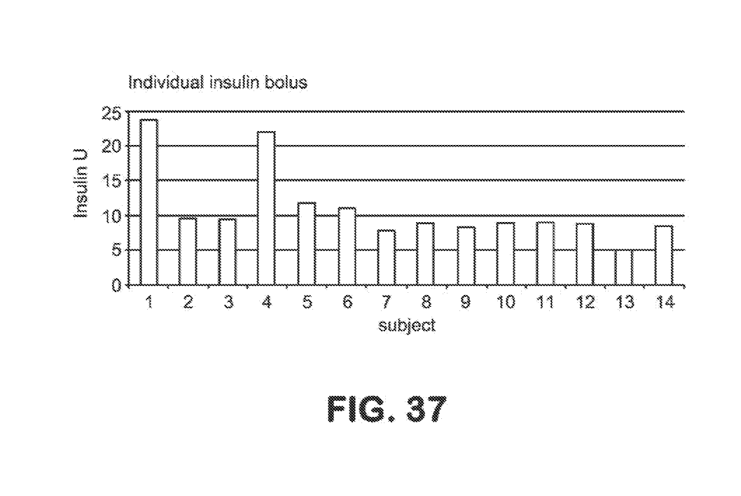

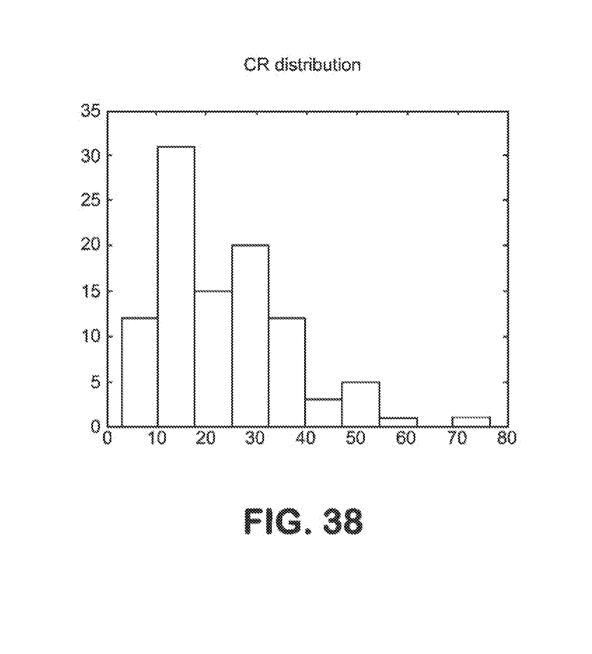

The data published in Woerle et al. (December, 2008) Diabetes Care 31(12): 2325-2331, where a dual tracer approach was used in healthy subjects to estimate the rate of appearance of glucose during the meal and hepatic glucose production during placebo/pramlintide were used in an aggregated fashion (individual data not available, FIG. 13A). We first demonstrated by using the glucose and C-peptide minimal models that pramlintide has no effect on insulin sensitivity and beta-cell function. Subsequently, the minimal model of gastrointestinal absorption (FIG. 37) was used to quantify the changes of the rate of appearance of ingested glucose due to pramlintide. We found that pramlintide decreases the parameter kmax (maximum gastric emptying by ⅔). This finding was incorporated into the Healthy State Simulator and predictions were successfully tested against Woerle et al data FIG. 38. We also successfully tested the prediction of 2 h postprandial glucose of the Type 1 Diabetes Simulator (illustrated in FIG. 18) (incorporating the new kmax) against the data by Kovatchev B P, et al., Pramlintide reduces the risks associated with glucose variability in type 1 diabetes. Diabetes Technol Ther. 2008 October; 10(5):391-6. The new model was finally incorporated into our model predictive control algorithm and in silico experiment on the Type 1 diabetes.

These in silico model development and experiments are an art-accepted model for pharmacokinetic/pharmacodynamic (PK/PD) modeling of pramlintide with associated inter-subject variability in the PK/PD parameters. This model was incorporated in the already existing type 1 diabetes simulator and virtual “patients”.

The in silico model was used to determine the efficacy and the safety (in terms of risk for hypoglycemia) of a broad range of fixed pramiintide:insulin ratios. The in silico trial identified three optimal ratios that should result in the best glucose (BG) regulation (maximum time spent within therapeutic range with minimum risk for hypoglycemia quantified by the Low BG Index and visualized by the Control Variability Grid Analysis). Using the simulator to incorporate PK/PD parameters of pramlintide, closed-loop model-predictive control strategies are run. Pre-meal conventional injections of pramlintide are simulated. The control algorithm is based on insulin-pramlintide action models that are suitably linearized and discretized for the purpose of control applications. See e.g., Kovatchev B. P, et al., “In Silico Preclinical Trials: A Proof of Concept in Closed-Loop Control of Type 1 Diabetes” Journal of Diabetes Science and Technology, Volume 3, Issue 1: Page 44-55, 2009; Woerle, et al., Importance of changes in gastric emptying for postprandial plasma glucose fluxes in healthy humans. Am. J. Physiol Endocrinol Metab. 2008 January; 294(1):E103-9(3); Cobelli C, et al., “Assessment of beta cell function in humans, simultaneously with insulin sensitivity and hepatic extraction, from intravenous and oral glucose test. Am J Physiol Endocrinol Metab, 293:E1-E15,2007; Dalla Man C., et al., “A system model of oral glucose absorption: validation on gold standard data” IEEE Trans Biomed Eng, 53:2472-8,2006; Kovatchev B P, et al., Pramlintide reduces the risks associated with glucose variability in type 1 diabetes. Diabetes Technol Ther. 2008 October; 10(5):391-6.

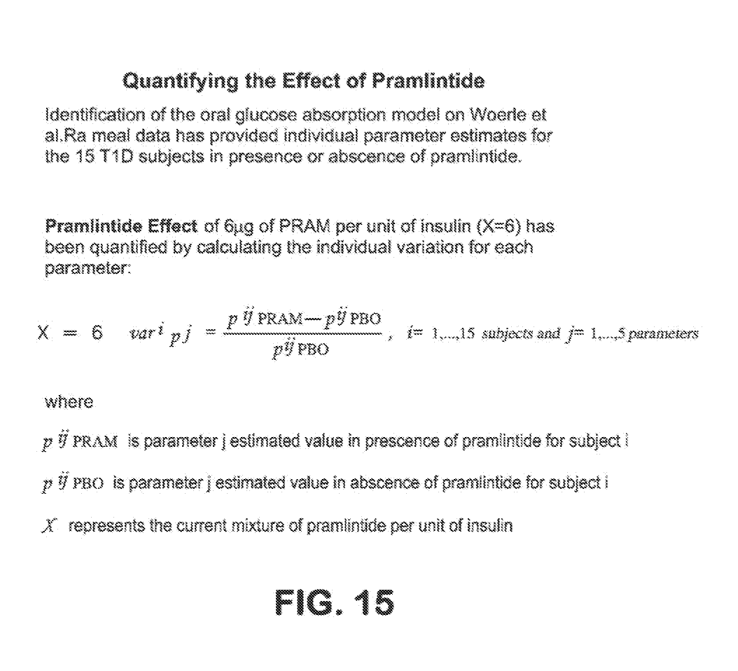

As illustrated in FIG. 15, the model of the gastro-intestinal tract on Rₐ meal data of the Woerle T1D database was used. The database was made up of 15 type 1 diabetic subjects (8 men and 7 women, 37±2 years of age, body weight 76±3 kg) (this is a database for each individual patient participating in the study published in Woerle et al. (December, 2008) Diabetes Care 31(12): 2325-2331, in which pramlintide was given to TI patients and labeled glucose appearance from meal to blood was measured; Woerle reported the effect of pramlintide on gastric emptying and thus glucose appearance in the blood after a meal in patients with type 1 diabetes). “Rₐ meal data” means the “glucose rate of appearance (Rₐ) of ingested glucose”; “Rₐ meal” refers to measurement in response to a meal (ingested), versus after say an intraduodenal injection of glucose.

Subjects were studied on 2 occasions: hyperglycemia with (PRAM) and without (PBO) 30 μg of pramlintide (Amylin Pharmaceuticals, San Diego, Calif.), injected subcutaneously in the lower abdominal wall with the standardized meal containing 50 g of glucose. Over the initial 90 min of the postprandial period, blood samples were taken at 15 min intervals and thereafter at 30-min intervals until completion of the experiment at 330 min.

The model has been numerically identified on placebo Ra meal data using nonlinear weighted least-squares estimator. Identification on pramlintide Ra meal data have required the use of Bayesian estimator in order to achieve a precise estimates of model parameters. Average and SD values of the model parameters estimates are reported in Table 1:

TABLE 1
Average and SD values of model parameters estimates.

| | | kmax | CV | kmin | CV | kabs | CV | b | CV | c | CV |
|---|---|---|---|---|---|---|---|---|---|---|---|
| PBO | mean | 0.071 | 33 | 0.008 | 32 | 0.066 | 49 | 0.766 | 9 | 0.170 | 37 |
| | SD | 0.062 | 25 | 0.002 | 21 | 0.059 | 22 | 0.176 | 7 | 0.156 | 15 |
| PRAM | mean | 0.041 | 49 | 0.005 | 43 | 0.020 | 44 | 0.985 | 2 | 0.537 | 27 |
| | SD | 0.026 | 23 | 0.002 | 20 | 0.013 | 23 | 0.033 | 3 | 0.080 | 11 |

Average model prediction against placebo (PBO) or pramlintide (PRAM) Ra meal data are shown in FIG. 13, where FIG. 13 graphically illustrates the average model prediction versus (vs) Ra meal data placebo (PBO) or pramlintide (PRAM).

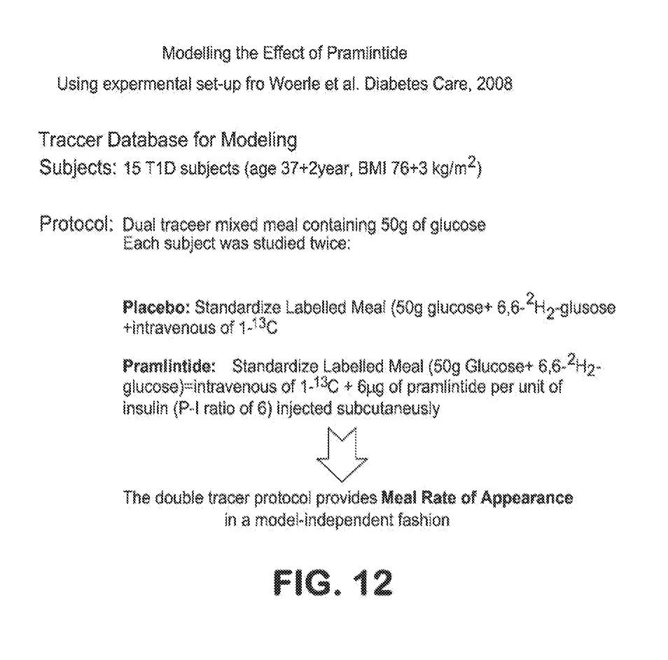

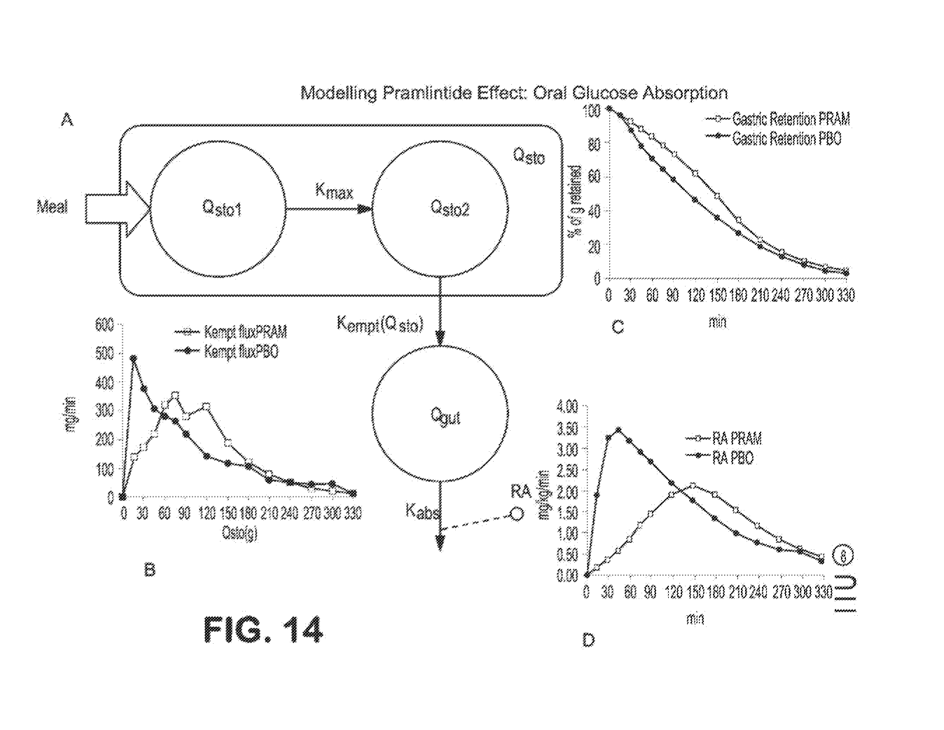

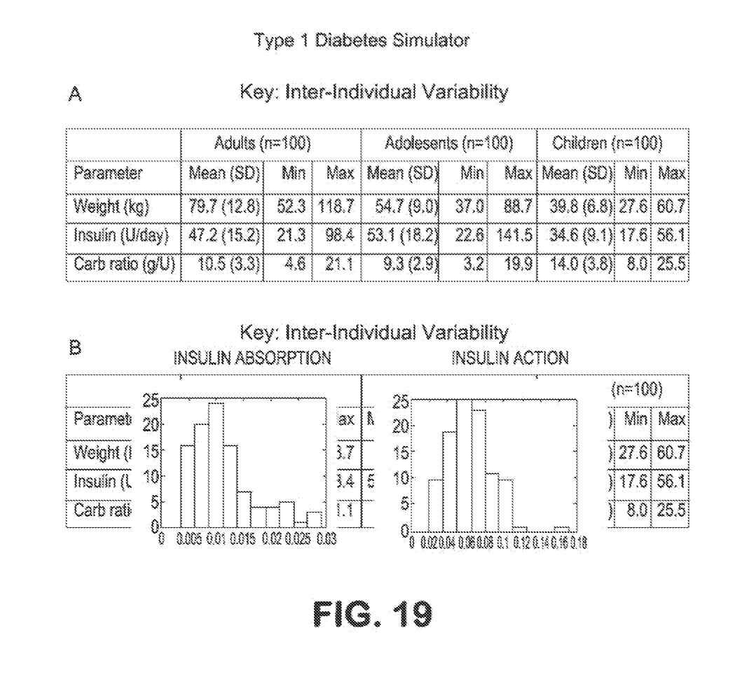

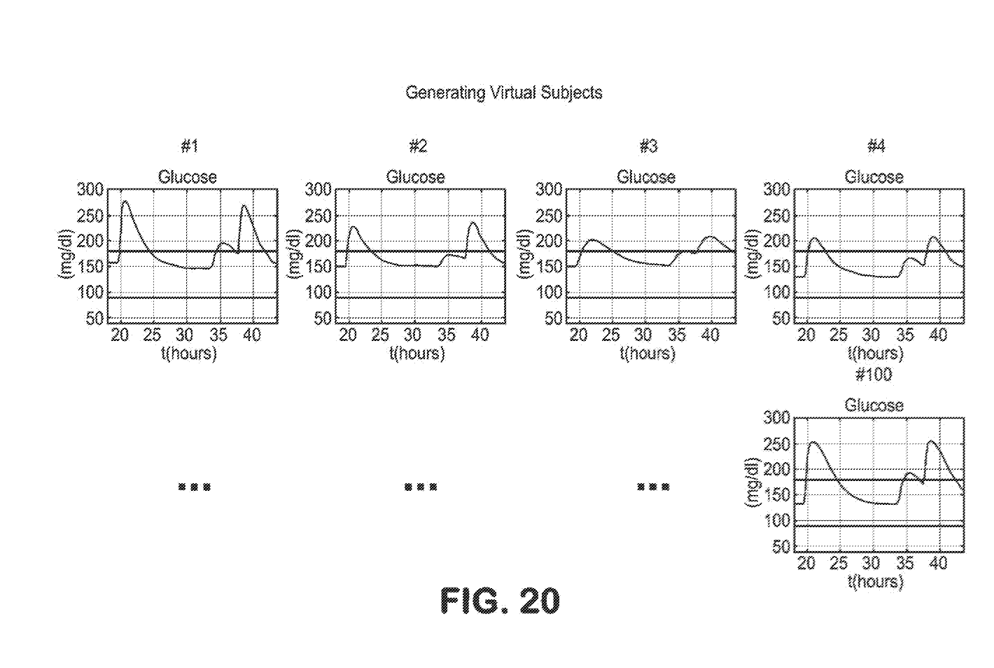

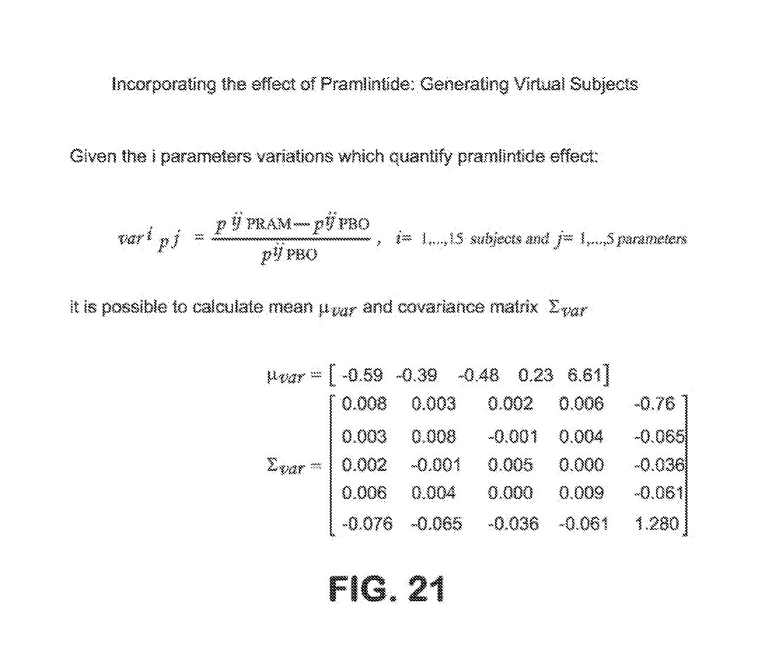

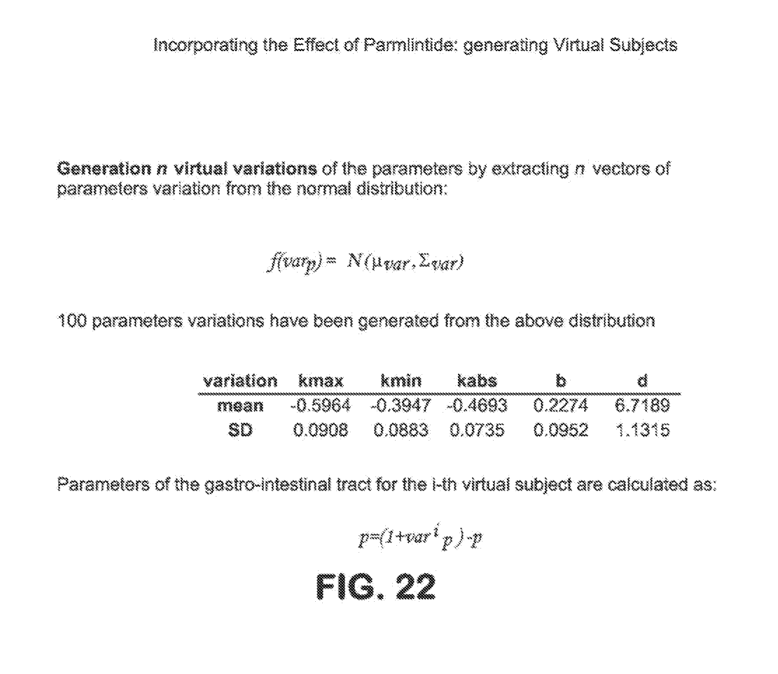

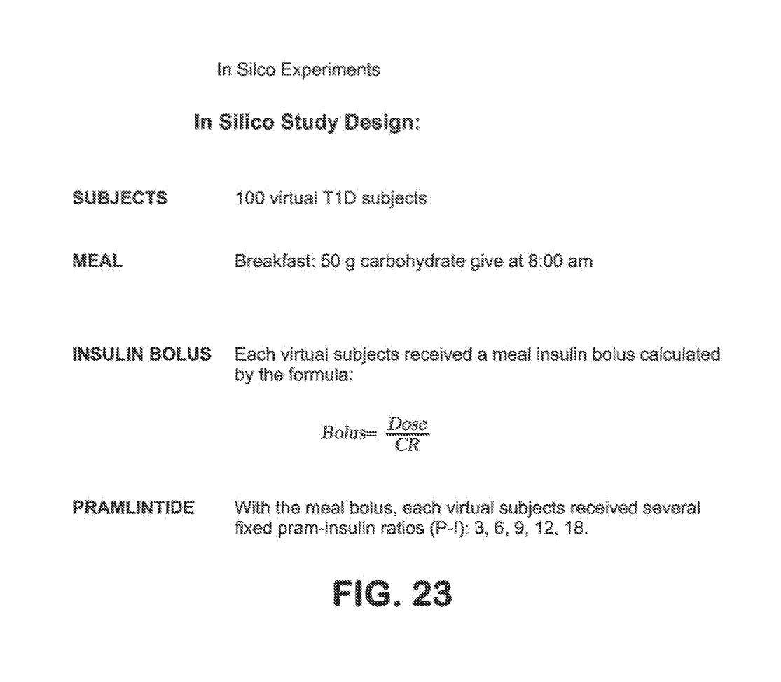

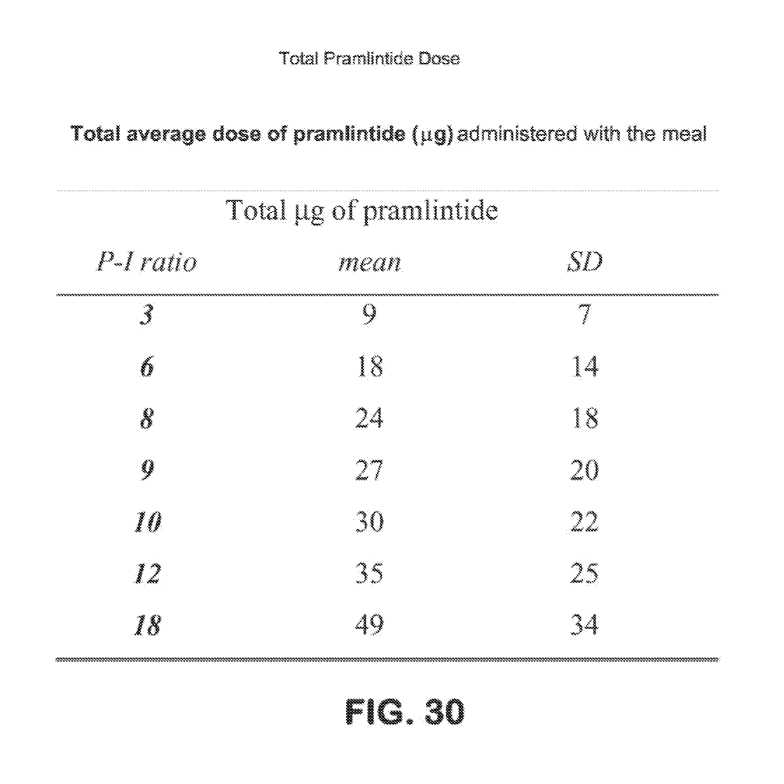

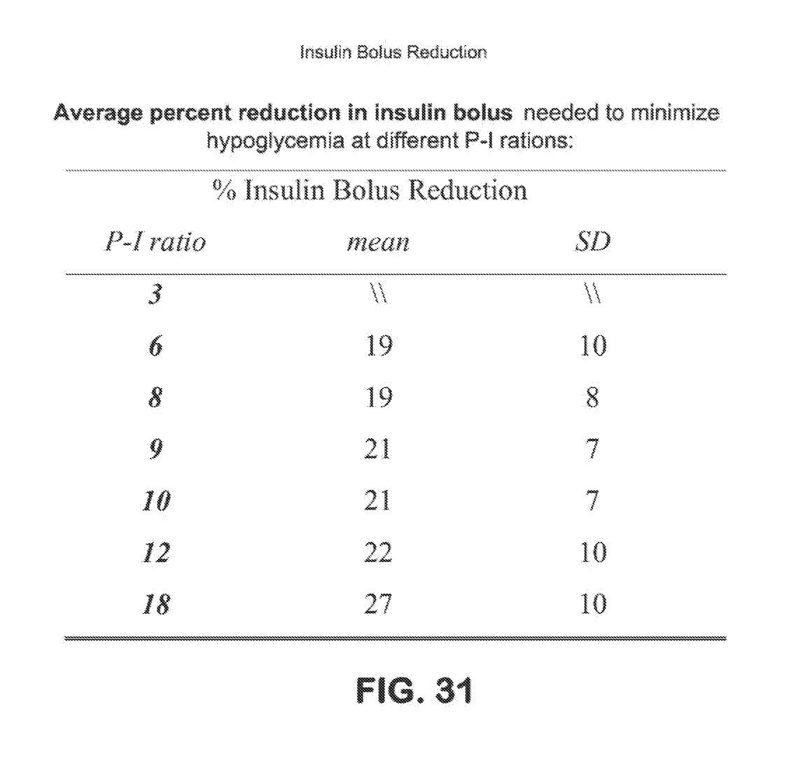

FIGS. 12 to 33 and 35 to 42 and FIG. 17, describe the in silico model, and the results of using that model, to determine the efficacy and the safety (in terms of risk for hypoglycemia) of a broad range of fixed pramlintide:insulin ratios of this invention. FIGS. 23 to 33 describe how the In Silico Experiments determined the optimal Pramlintide-Insulin Ratios of this invention, including the exemplary P:I ratio of 9:1.

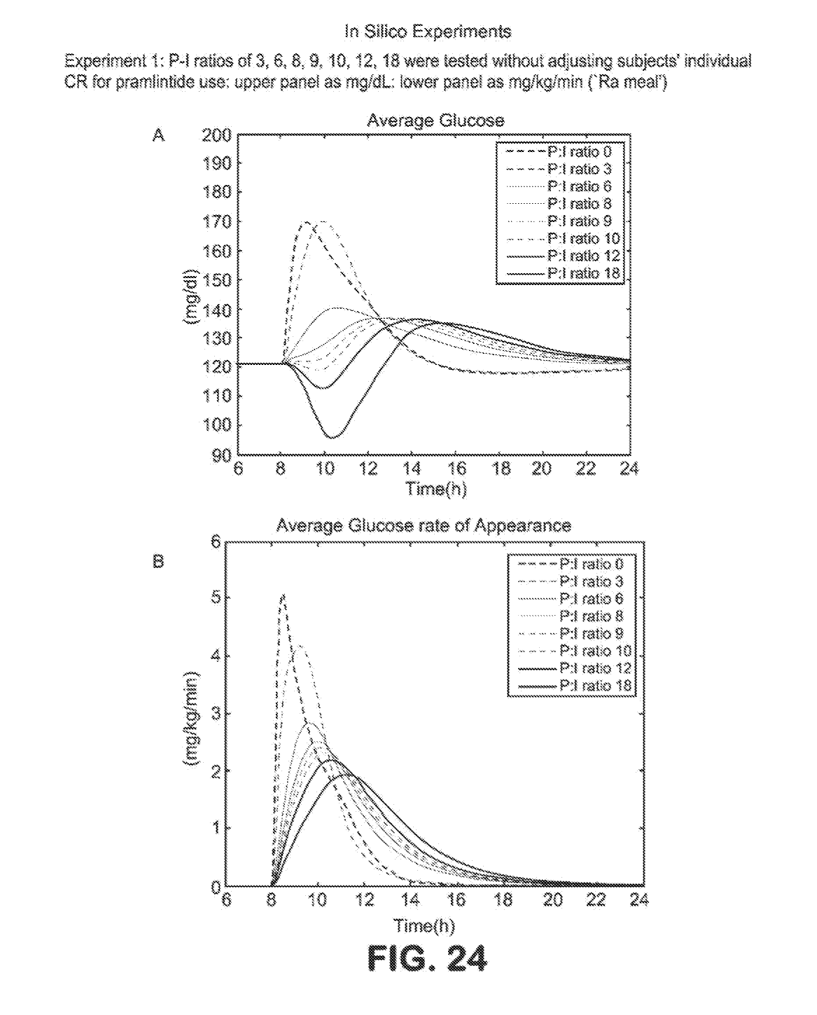

In Experiment 1, different P-I ratios' (illustrated in FIG. 24) efficacy in attenuating postprandial hyperglycemia and safety in terms of hypoglycemia were evaluated by Control Variability-Grid Analysis (CVGA). In FIG. 24, P:I ratios of 3, 6, 8, 9, 10, 12 and 18 were tested without adjusting the subjects' individual CR for pramlintide use, FIG. 24A, the upper panel, in mg/dL; FIG. 24B, the lower panel, as mg/kg/min (the “Ra meal”).

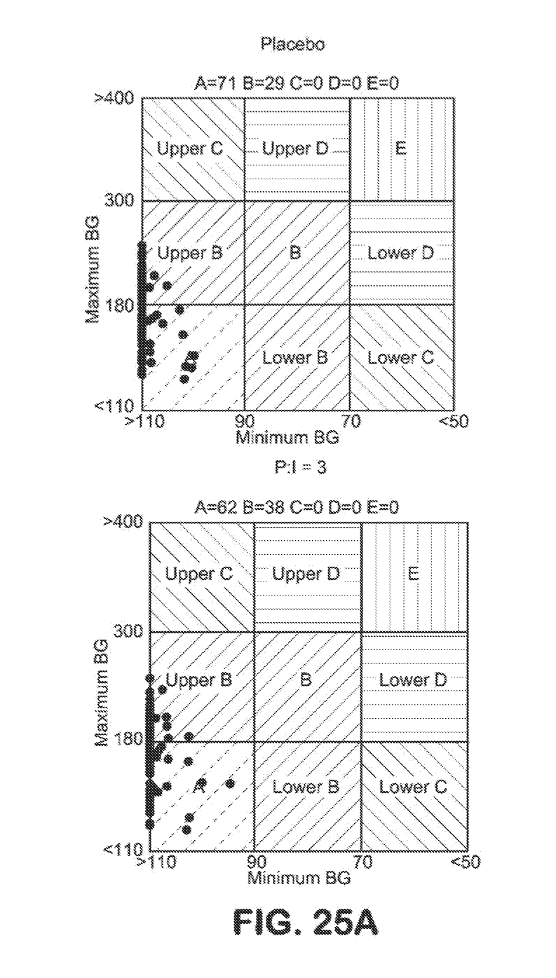

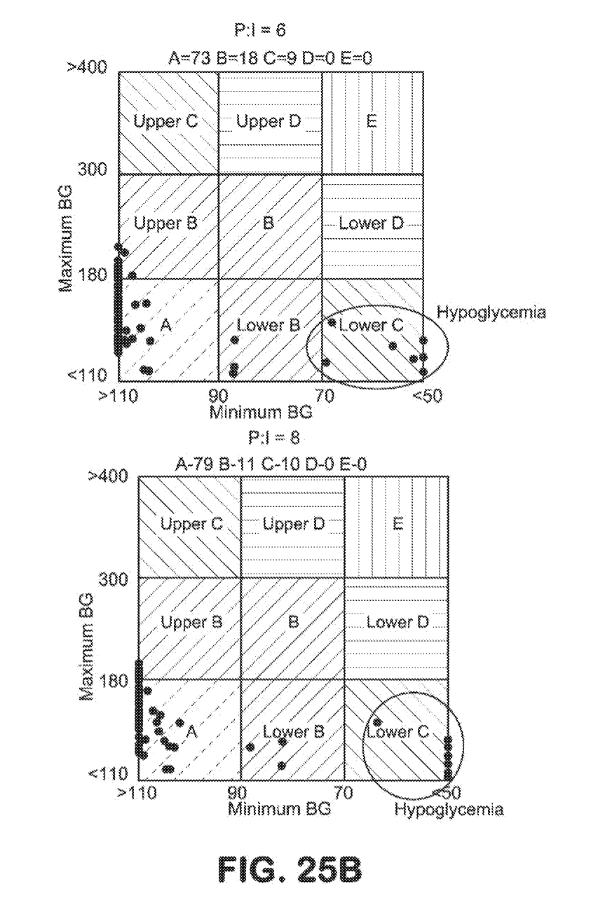

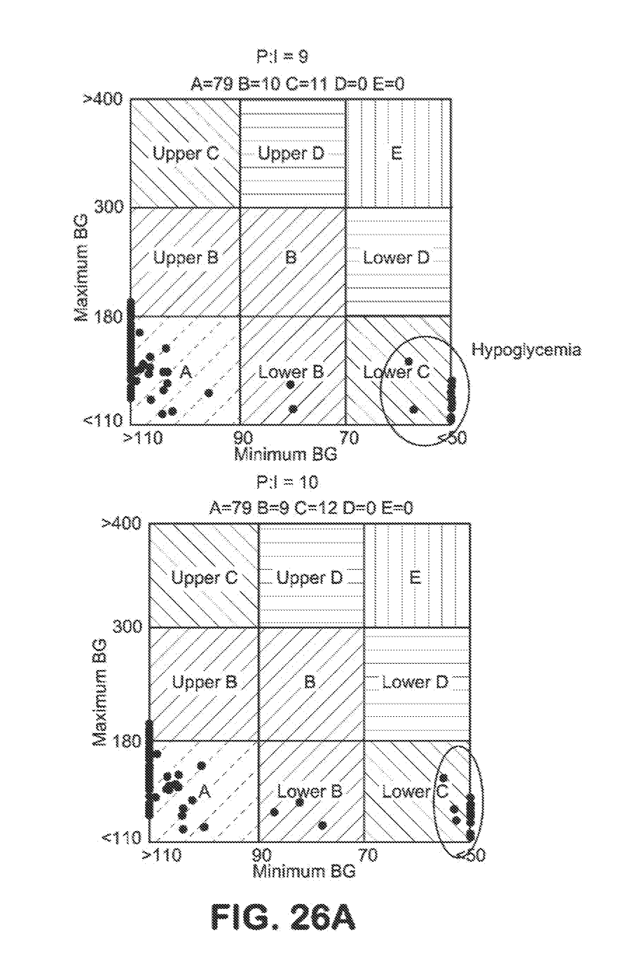

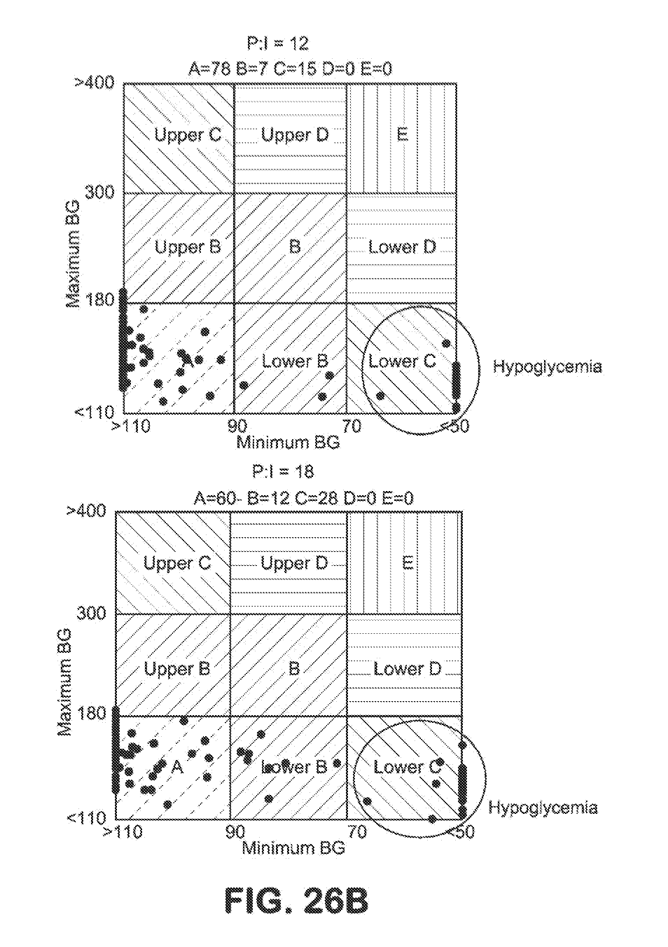

The conclusion of Experiment 1 (without adjusting the subject's individual carbohydrate ratio), with data is illustrated in FIGS. 25 and 26, are: P-I ratio of 3 is not better than insulin alone (Placebo); it only delays the postprandial blood glucose peak, but does not attenuate its magnitude. Without adjusting subjects individual CR for pramlintide use, P-I ratios of 6, 8, 9, 10, 12 and 18 resulted in increased hypoglycemia, with 9%, 10%, 11%, 12%, 15% and 28% of subjects experiencing glucose levels <50 mg/dl.

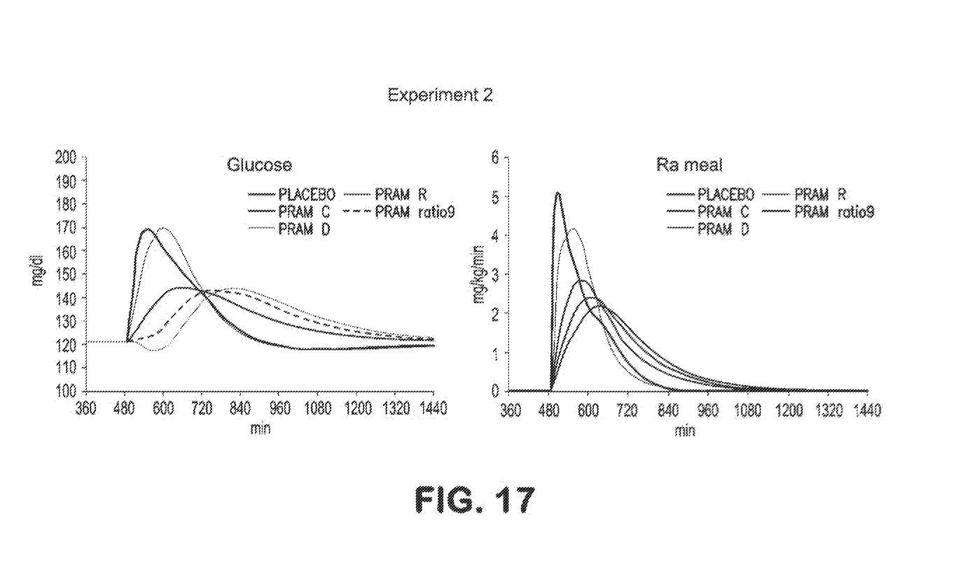

FIG. 26A graphically illustrates Control Variability-Grid Analysis (CVGA) for the 100 virtual subjects in case pramlintide/insulin bolus ratio 9:1. Hypoglycemic event occur in 11% (2 more than case C and 4 less than case D) of the subjects, hence a new insulin bolus has been calculated by increasing CR, until a new optimum CR has been found (average percent CR increase is less than 30%). Then the simulations have been performed again with adjusted CR. Results are shown in FIG. 17, which graphically illustrates the average and individual glucose plasma concentration and Ra meal for the 100 virtual subjects in case pramlintide/insulin bolus ratio 9 with adjusted CR.

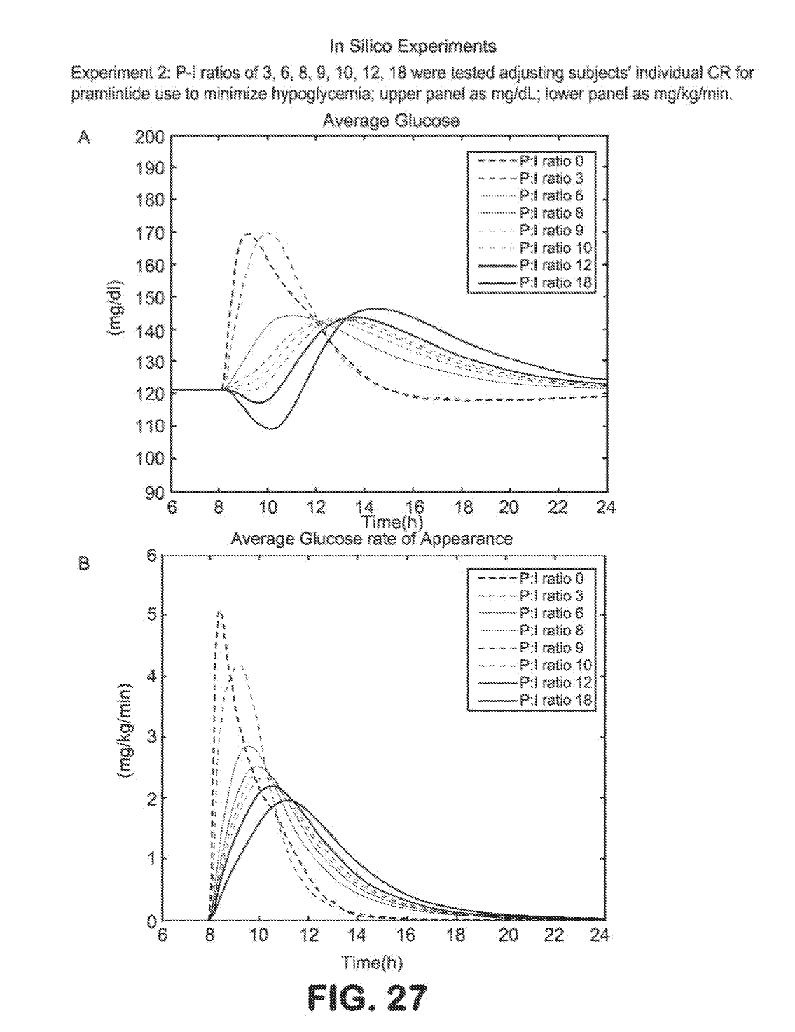

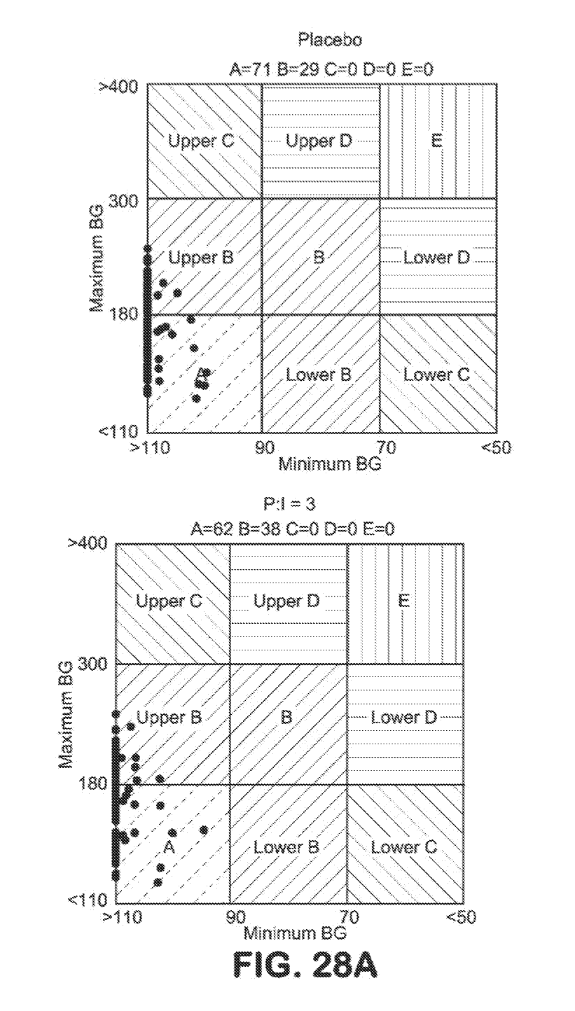

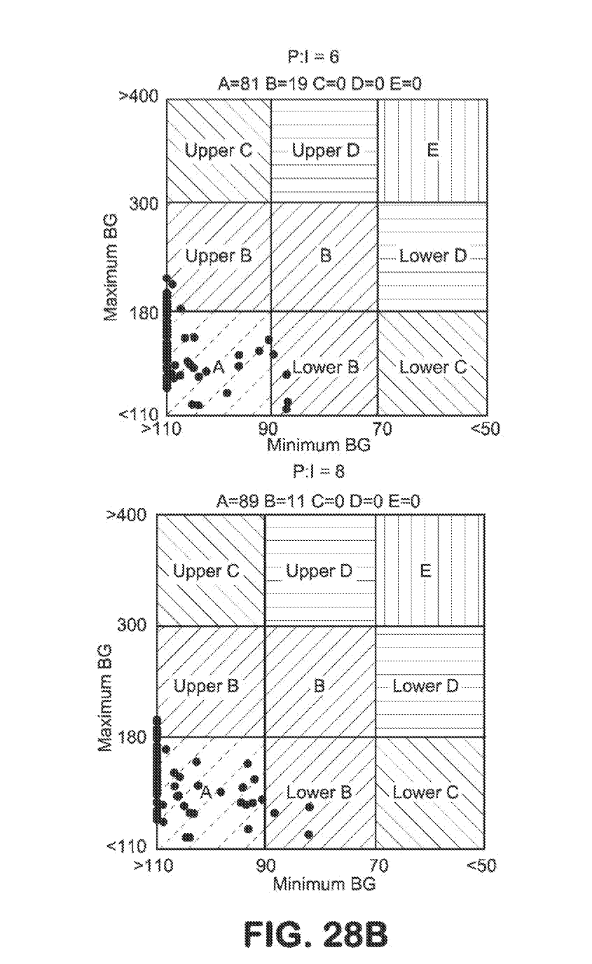

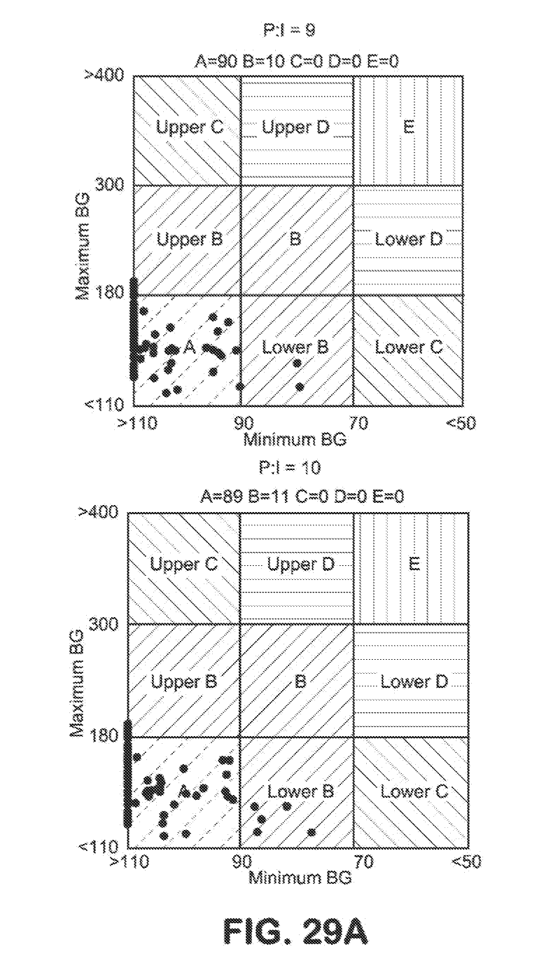

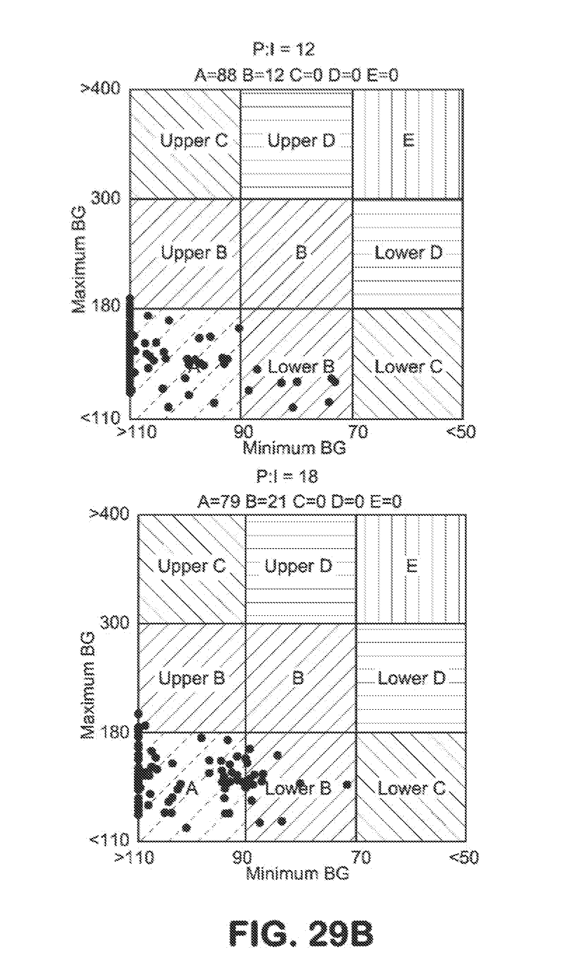

Experiment 2, adjusting for subject's individual carbohydrate ratio, is illustrated in FIG. 27, with data shown in FIGS. 27, 28 and 29 and FIGS. 41 A and 41B. In FIG. 27, P:I ratios of 3, 6, 8, 9, 10, 12 and 18 were tested adjusting the subjects' individual CR for pramlintide use to minimize hypoglycemia, FIG. 27A, the upper panel, in mg/dL; FIG. 27B, the lower panel, as mg/kg/min.

Results showed that a P-I ratio of 3 was no more efficient than placebo with 62% vs. 71% in A-zone; and P-I ratios of 6, 8, 9, 10, 12 and 18 resulting in significant improvement of postprandial glucose control: 81%, 89%, 90%, 89%, 88% and 79% of subjects in CVGAA-zone, respectively, and no hypoglycemia.

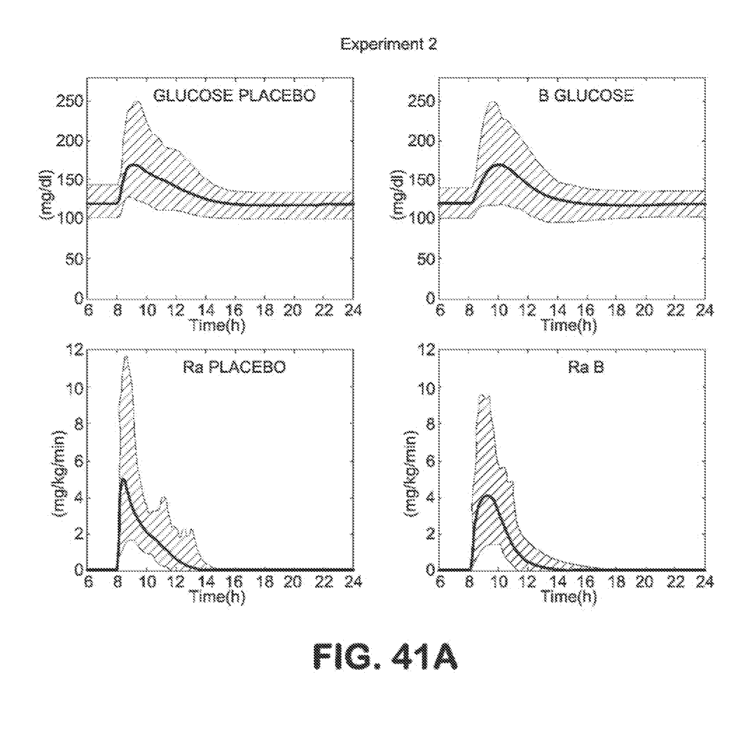

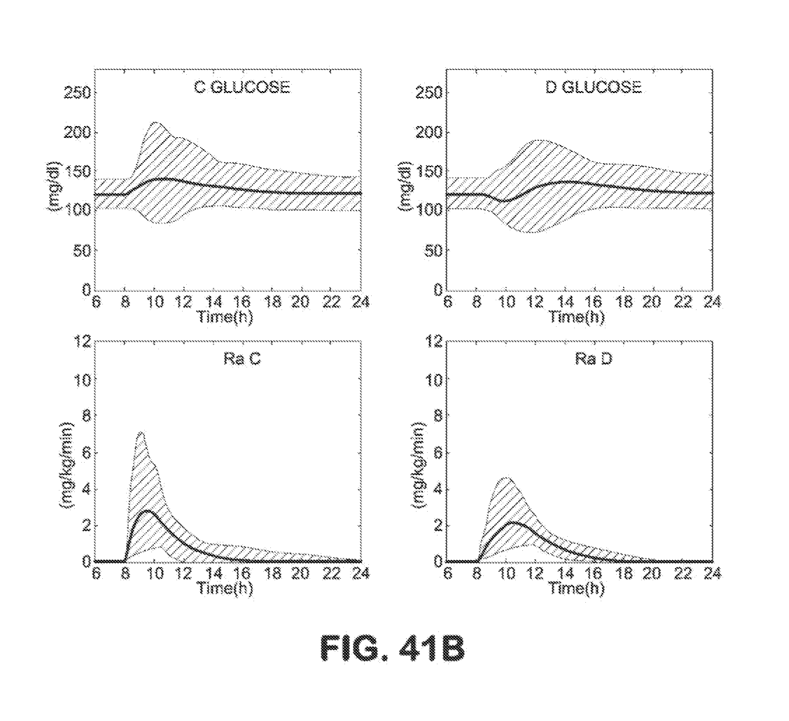

FIG. 27 illustrates data from: P-I ratios of 3, 6, 8, 9, 10, 12, 18 were tested adjusting subjects individual CR for pramlintide use to minimize hypoglycemia; upper panel as mg/dL; lower panel as mg/kg/min. FIGS. 28A and 28B, graphically illustrate Average and individual glucose plasma concentration and Ra meal for the 100 virtual subjects in case A, B, C and D with adjusted CR. FIGS. 28A and 28B graphically illustrates CVGA for the 100 virtual subjects in case A, B, C and D with adjusted CR. Where the Carbohydrate grams to Insulin Units ratio (CR) is adjusted based on a lower insulin need per carbohydrate when pram is present. FIG. 29A graphically illustrates CVGA for the 100 virtual subjects in case pramlintide/insulin bolus ratio 9 with adjusted CR. FIGS. 41 A and 41B graphically illustrate average and individual glucose plasma concentration and Ra meal for the 100 virtual subjects in case A, B, C and D with adjusted CR.

The data from these in silico studies allow the conclusion that in the clinic: Pramlintide dose should be adjusted in parallel with patients' carbohydrate ratio; average CR reduction of 20% appears warranted; and a P-I ratio of 9 was demonstrated to be optimal in terms of pramlintide efficacy and safety.

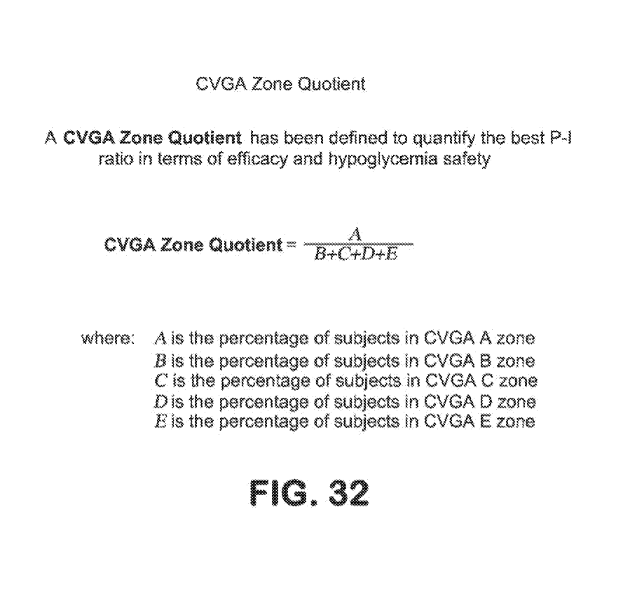

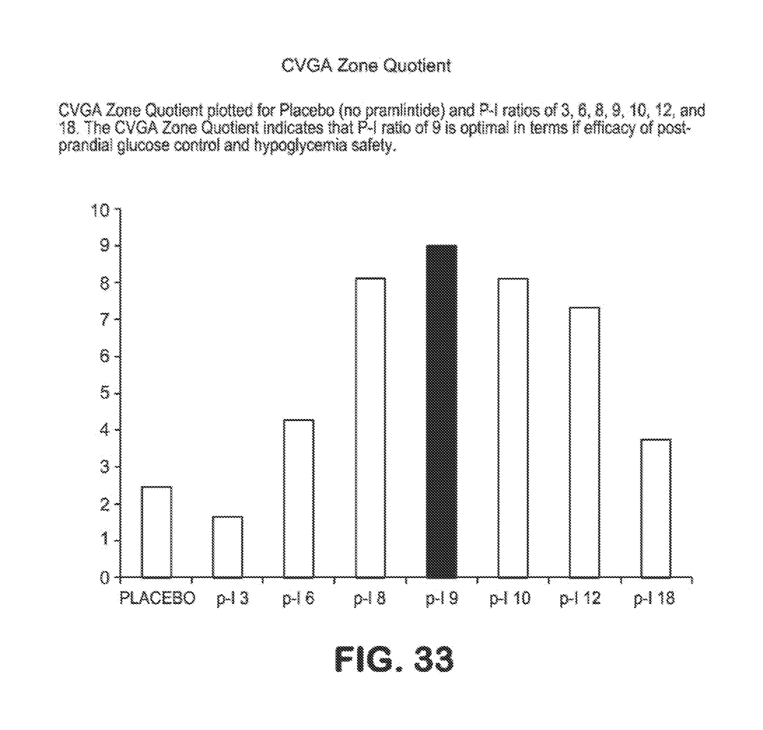

FIGS. 32 and 33 illustrate data from Control Variability-Grid Analysis (CVGA); different P-I ratios efficacy in attenuating postprandial hyperglycemia and safety in terms of hypoglycemia was evaluated by Control Variability-Grid Analysis (CVGA). Results showed: P-I ratio of 3 is not better than insulin alone (Placebo); it only delays the postprandial blood glucose peak, but does not attenuate its magnitude; without adjusting subjects individual CR for pramlintide use, P-I ratios of 6, 8, 9, 10, 12 and 18 resulted in increased hypoglycemia, with 9%, 10%, 11%, 12%, 15% and 28% of subjects experiencing glucose levels <50 mg/dl. In summary, in FIGS. 32 and 33, results were that P-I ratio of 3 were no more efficient than placebo with 62% vs. 71% in A-zone; and, P-I ratios of 6, 8, 9, 10, 12 and 18 resulting in significant improvement of postprandial glucose control: 81%, 89%, 90%, 89%, 88% and 79% of subjects in CVGAA-zone, respectively, and no hypoglycemia.

The data from these in silico studies allow the conclusion that in the clinic: Pramlintide dose should be adjusted in parallel with patients' carbohydrate ratio; average CR reduction of 20% appears warranted; and P-I ratio of 9:1 is likely to be optimal in terms of pramlintide efficacy and safety.

Single-Meal in Silico Scenario

A single-meal in silico scenario was also used, and it demonstrated:
Pramlintide/insulin bolus ratio of 3 is not better than insulin alone—it only delays the postprandial blood glucose peak, but does not attenuate its magnitude.
Pramlintide/insulin of 6 and 12 have visible effect, but require adjustment of carb ratio by 20% and 30% on average to avoid hypoglycemia.
After appropriate adjustment, both 6 and 12 ratios result in significant improvement of postprandial glucose control. A ratio of 12 is better than 6, but might increase hypoglycemia; the incremental improvement in terms of reduced hyperglycemia is not dramatic.
As a result, we can hypothesize that a ratio of 9 would be optimal (this can be of course tested directly in an additional simulation).

Insulin Bolus Reconstruction

In order to quantify pramlintide (Vg)/insulin bolus (U) ratio used in Woerle protocol, insulin rate of appearance into plasma has been reconstructed by deconvolution where average insulin plasma concentration data have been used as output and the system has been modeled with a single exponential whose parameters have been fixed to population values (Campioni et al. 2009).

Using data of infused insulin allowed direct measurement of insulin bolus by calculating area under the curve (AUC) of the infused insulin minus AUC of the basal infusion. Insulin infusion units reported were IE/h, which has been considered to be equivalent to U/h due to observed insulin values range. Individual calculated insulin bolus (U) is shown in FIG. 37. The average given insulin bolus is equal to 10.93 U, leading to a pramlintide/insulin bolus ratio equal to 2.73.

The average given bolus reconstructed is equal to 16.5 U, hence the P:I, or pramlintide/insulin, bolus ratio is equal to 1.82. We calculated insulin bolus using the formula:

$$Bolus = \frac{Dose}{CR} ,$$

with CR extracted from CR distribution shown in FIG. 38 and equal to 10 g/U.

Calculated bolus is then equal to 5 U, hence pramlintide/insulin bolus ratio is equal to 6. We concluded that pramlintide/insulin bolus ratio adopted in the Woerle protocol is equal to 6.

Generation of Virtual Parameter Variation

Identification of the gastro-intestinal tract model on Woerle Ra meal data has provided individual parameters estimates for the 15 T1D subjects in presence or absence of pramlintide. Then individual variation for each parameter has been calculated as:

$${var}_{{p}^{j}}^{i} = \frac{{p}_{PRAM}^{ij} - {p}_{PBO}^{ij}}{{p}_{PBO}^{ij}} , i = 1 , … , 15 subjects and$$

$$j = 1 , … , 5 parameters$$

where p^{ij}_{PRAM} is parameter j estimated value in presence of pramlintide and p^{ij}_{PBO} is parameter j estimated value in absence of pramlintide for subject i.

Then given the i variations it is possible to calculate mean and covariance matrix, μᵥₐᵣ and Σᵥₐᵣ respectively:

$${μ}_{va r} = [ \begin{matrix}- 0.59 & - 0.39 & - 0.48 & 0.23 & 6.61\end{matrix} ]$$

$${Σ}_{va r} = [ \begin{matrix}0.076 & 0.041 & 0.003 & 0.061 & - 0.673 \\ 0.041 & 0.099 & - 0.022 & 0.045 & - 0.690 \\ 0.003 & - 0.022 & 0.054 & - 0.016 & - 0.286 \\ 0.061 & 0.045 & - 0.016 & 0.094 & - 0.512 \\ - 0.673 & - 0.690 & - 0.286 & - 0.512 & 12.900\end{matrix} ]$$

Given μᵥₐᵣ and Σᵥₐᵣ is then possible to generate n virtual variations of the parameters by extracting n vectors of parameters variation from the normal distribution:

f(varₚ)=N(μᵥₐᵣ,Σᵥₐᵣ)

We reduced the standard deviations of the variation of the parameters to the 30% of their values due to the small number of subjects available:

$${Σ}_{va r}^{0.3} = [ \begin{matrix}0.008 & 0.003 & 0.002 & 0.006 & - 0.076 \\ 0.003 & 0.008 & - 0.001 & 0.004 & - 0.065 \\ 0.002 & - 0.001 & 0.005 & 0.000 & - 0.036 \\ 0.006 & 0.004 & 0.000 & 0.009 & - 0.061 \\ - 0.076 & - 0.065 & - 0.036 & - 0.061 & 1.280\end{matrix} ]$$

Then 100 virtual parameters variations were generated from the distribution:

f(varₚ)=N(μᵥₐᵣ,Σ^{0.3}ᵥₐᵣ)

| variation | kmax | kmin | kabs | b | d |
|---|---|---|---|---|---|
| mean | −0.5964 | −0.3947 | −0.4693 | 0.2274 | 6.7189 |
| SD | 0.0908 | 0.0883 | 0.0735 | 0.0952 | 1.1315 |

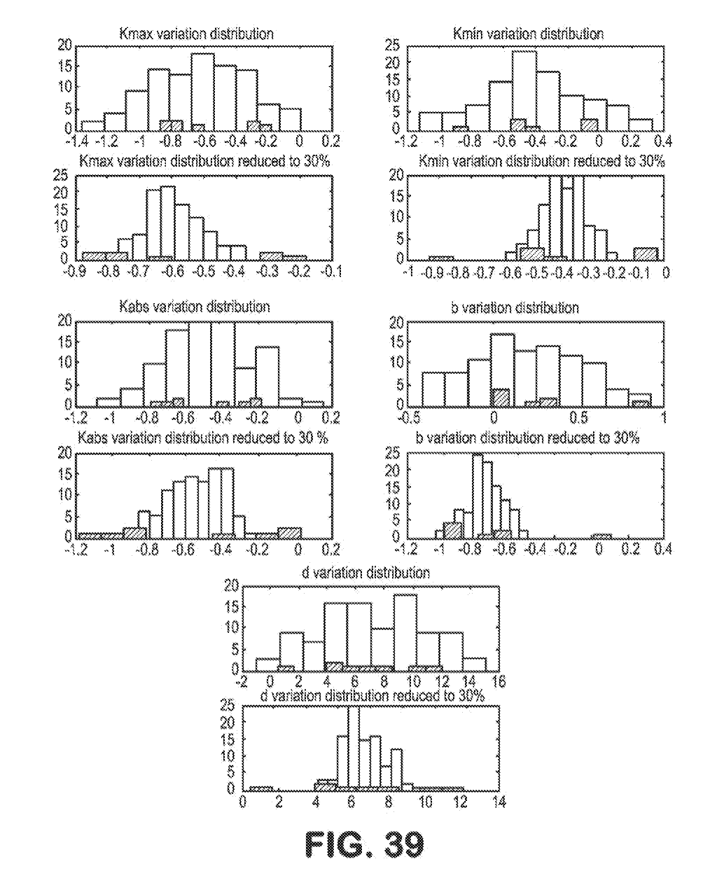

FIG. 39 illustrates several parameters variations distributions, wherein blue (the darker illustrated graphic) distribution are relative to parameters variations calculated from the database's estimated parameters; and the green (the lighter illustrated graphic) distribution are relative to virtual parameters variations.

Modeling Pramlintide Dose-Response

Effect of pramlintide was modeled as follows:

given X the current pramlintide/insulin bolus ratio if X=6 then the parameters variations of the i-th subject varⁱₚ are exactly those generated from the distribution f(varₚ)=N(μᵥₐᵣ,Σ^{0.3}ᵥₐᵣ), since we have the same conditions of Woerle protocol form which parameters variations have been calculated.

If X<6 then the parameters variations of the i-th subject are:

$${var}_{p}^{i} = \frac{{var}_{p}^{i}}{\frac{6}{X}}$$

e.g. with X=3 parameters variations is halved if compared to X=6.

If X>6 then the parameters variations of the i-th subject are:

$${var}_{p}^{i} = {var}_{p}^{i} max - \frac{{var}_{p}^{i} max - {var}_{p}^{i}}{\frac{X}{6}}$$

where varⁱₚmax are the maximum variations for the parameters, e.g. parameter b has a maximum value equal to 1, hence its

$${var}_{p}^{i} max = \frac{1 - b}{b} .$$

Then parameters p=[kmax kmin kabs b d] of the gastro-intestinal tract for the i-th subject are calculated as p=(1+varⁱₚ)·p

The dose-response curve has been calculated by spanning several pramlintide/insulin bolus ratio:

X=[0.01, 0.05, 0.1, 0.2, 0.4, 0.8, 0.9, 1, 2, 3, . . . , 24] on the average subject and then plotting on x axis the values of X and on y axis the percentage of glucose retained in the stomach at 120 min.

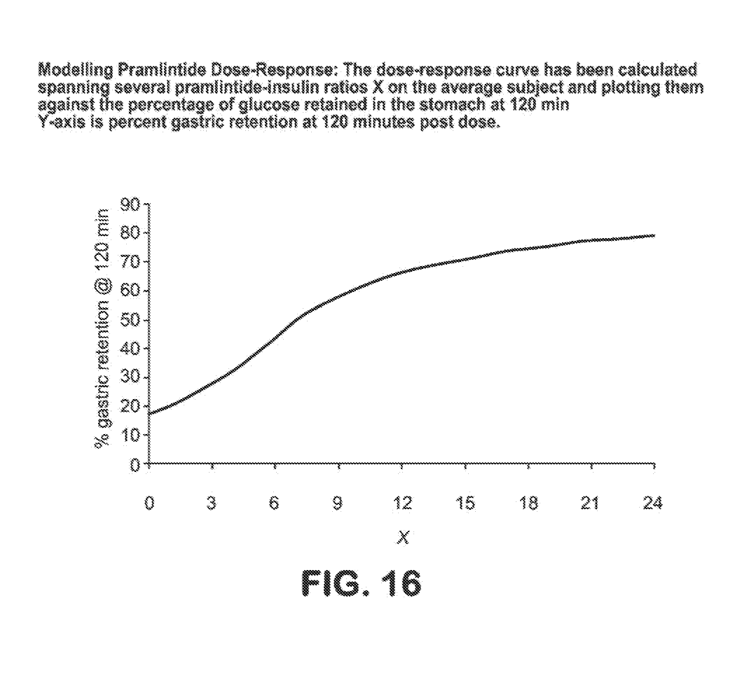

FIG. 16 graphically illustrates the Dose-response curve for the average subject.

Simulation in silico of Pramlintide Adaptive Clinical Trial

We in silico simulated the effect of 3 fixed pramlintide/insulin bolus ratio B, C, D: X=[3, 6, 12] on Ra meal and postprandial glucose in 100 virtual T1D subjects and compared with placebo. The Simulation scenario is a breakfast composed of 50 g of glucose given at 8.00 am. For placebo, case A, virtual subjects received together with the meal an insulin bolus calculated with the formula:

$$Bolus = \frac{Dose}{CR}$$

where CR is calculated for each subject using the following definition: optimal CR makes minimum G, reached after a 70 g meal, to be equal to Gb with a tolerance of 5% (i.e. 0.95Gb<Gmin<1.05Gb).

In case B, C and D subjects received together with the meal and the insulin bolus (equal to the one given in case A) the correct amount of Pramlintide in order to have the desired pramlintide/insulin bolus ratio, 3, 6, 12 respectively. Basal glucose has been lowered for each virtual subject of 20 mg/dl.

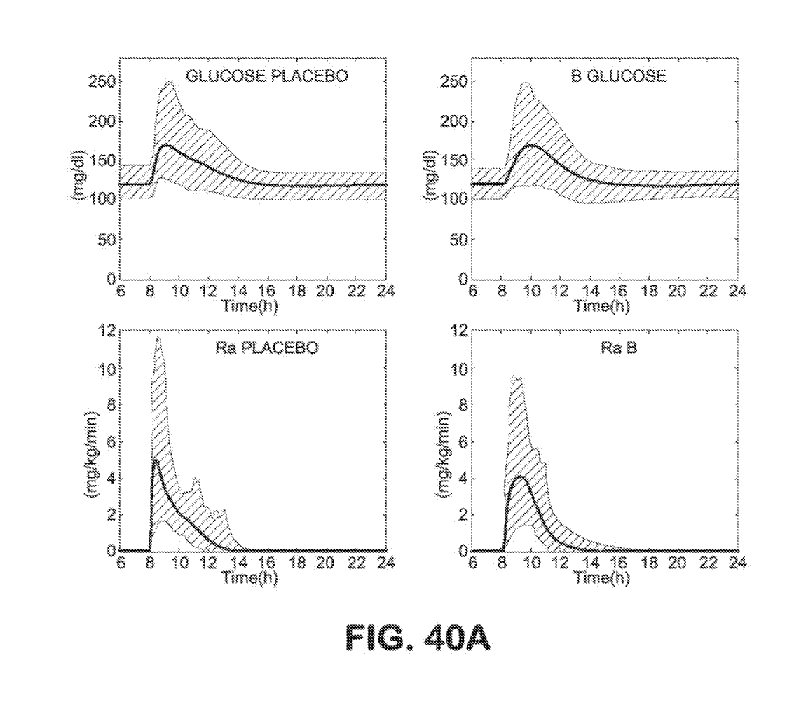

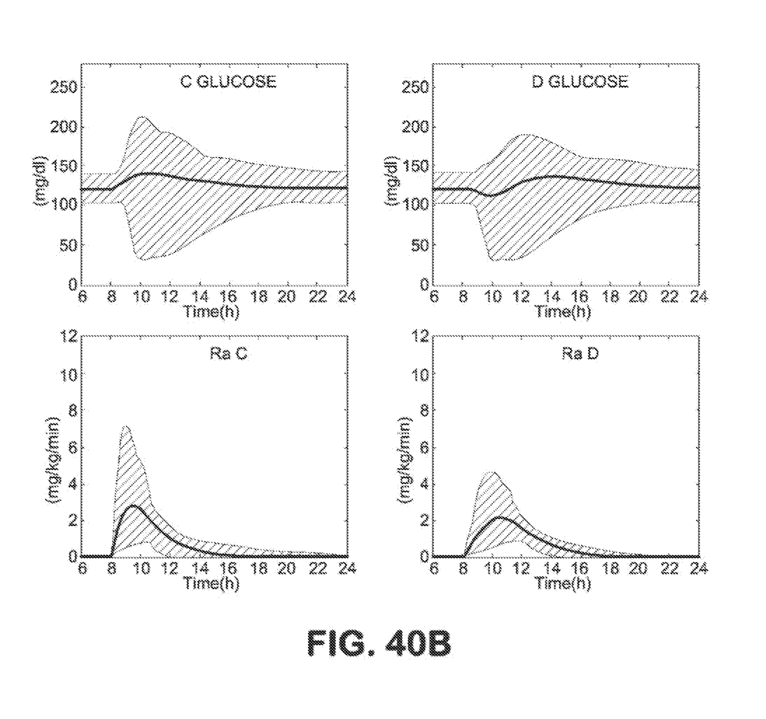

FIGS. 40A and 40B graphically illustrates the average and individual glucose plasma concentration and Ra meal for the 100 virtual subjects in case A, B, C and D; where A is placebo, B is a P:I of 3, C is a P:I of 6, and D is a P:I of 12. FIG. 24 (Experiment 1) graphically illustrates CVGA for the 100 virtual subjects in case A, B, C and D.

For those subjects which hypoglycemic event occur (9% in case C and 15% in case D), a new insulin bolus has been calculated by increasing CR, until a new optimum CR has been found (average percent CR increase in case C is 20% while in case D is about 30%).

In conclusion, for a single-meal scenario, these in silico studies demonstrate:
Pramlintide/insulin bolus ratio of 3 is not better than insulin alone—it only delays the postprandial blood glucose peak, but does not attenuate its magnitude.
Pramlintide/insulin of 6 and 12 have visible effect, but require adjustment of carb ratio by 20% and 30% on average to avoid hypoglycemia.
After appropriate adjustment, both 6 and 12 ratios result in significant improvement of postprandial glucose control. A ratio of 12 is better than 6, if it can be tolerated by the patient, but the incremental improvement is not dramatic.
As a result, we can hypothesize that a ratio of 9 would be optimal (this can be of course tested directly in an additional simulation).

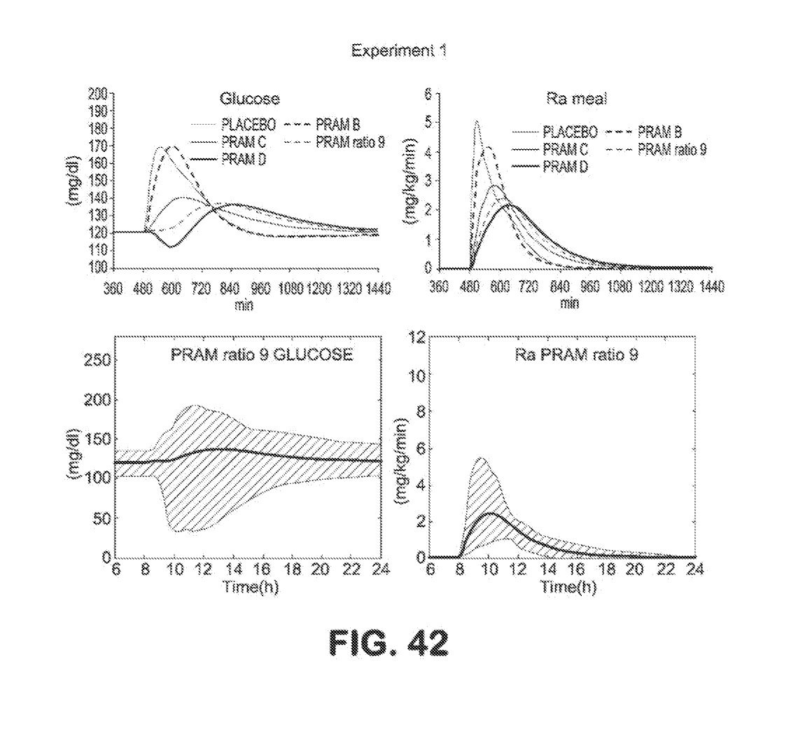

Simulations results obtained with the three different pramlintide/insulin ratio, X=[3, 6, 12], pointed out that a pramlintide/insulin ratio of 9 could be optimal, hence we have performed the simulations with X=[9]. Simulated Glucose plasma concentration and RA meal for X=[9] without CR adjustment are shown in FIG. 42 and marked in black, wherein FIG. 42 graphically illustrates the average and individual glucose plasma concentration and Ra meal for the 100 virtual subjects in case pramlintide/insulin bolus ratio 9.

A number of embodiments of the invention have been described. Nevertheless, it will be understood that various modifications may be made without departing from the spirit and scope of the invention. Accordingly, other embodiments are within the scope of the following claims.

1: A liquid pharmaceutical composition or a formulation, or a reconstitutable dried pharmaceutical composition or formulation, comprising:
(a) (i) a pramlintide or a pramlintide peptide, or a physiologically acceptable salt thereof; and
(ii) a human insulin or a human insulin peptide (HIP) or an analog thereof, or a physiologically acceptable salt thereof,
and optionally the human insulin, human insulin peptide (HIP), or analog thereof is or comprises: an aspart, a NOVOLOG™ or a NOVORAPID™ (Novo Nordisk, Bagsværd, Denmark); a glulisine or an APIDRA™ (Sanofi S.A., Paris, France); a lispro, an insulin lispro protamine or a HUMALOG™ (Eli Lilly and Company, Indianapolis, Ind.); a HUMULIN R™, a HUMULIN N™, a HUMULIN 70/30™ or a HUMULIN 70/30™ (Eli Lilly and Company, Indianapolis, Ind.);
or a regular (wild type) isolated or a recombinant human insulin, or a fast-acting human insulin analog or variant thereof,
and optionally the pramlintide peptide comprises or consists of a C-terminal amide form of a peptide
(SEQ ID NO: 1)
KCNTATCATQRLANFLVHSSNNFGPILPPTNVGSNTY;
wherein the ratio of the pramlintide or pramlintide peptide to the human insulin, human insulin peptide (HIP) in the liquid, reconstitutable dried pharmaceutical composition or formulation is:
4 μgm:1 U; or 5.92 mole insulin to 1 mole pramlintide;
4.5 μgm:1 U; or 5.26 mole insulin to 1 mole pramlintide;
5 μgm:1 U; or 4.74 mole insulin to 1 mole pramlintide;
5.5 μgm:1 U; or 4.31 mole insulin to 1 mole pramlintide;
6 μgm or 1.52 nmoles pramlintide:1 U (international unit) or 6.0 nmoles human insulin or equivalent nmoles of human insulin peptide providing 1 U of insulin activity, or 0.25 mole pramlintide to 1 mole human insulin (6 μgm:1 U) or moles of human insulin peptide providing the same unit of activity as 0.25 mole human insulin, or 3.98 mole human insulin or equivalent moles of human insulin peptide to 1 mole pramlintide;
6.5 μgm:1 U; or 3.63 mole insulin to 1 mole pramlintide;
7 μgm:1 U; or 3.38 mole insulin to 1 mole pramlintide;
8 μgm:1 U; or 2.96 mole insulin to 1 mole pramlintide;
8.5 μgm:1 U; or 2.79 mole insulin to 1 mole pramlintide;
9 μgm:1 U; or 2.63 mole insulin to 1 mole pramlintide;
9.5 μgm:1 U; or 2.49 mole insulin to 1 mole pramlintide;
10 μgm:1 U; or 2.37 mole insulin to 1 mole pramlintide;
11 μgm:1 U; or 2.15 mole insulin to 1 mole pramlintide;
12 μgm:1 U; or 1.97 mole insulin to 1 mole pramlintide;
13 μgm:1 U; or 1.82 mole insulin to 1 mole pramlintide;
14 μgm:1 U; or 1.69 mole insulin to 1 mole pramlintide;
15 μgm:1 U; or 1.58 mole insulin to 1 mole pramlintide;
16 μgm:1 U; or 1.48 mole insulin to 1 mole pramlintide;
17 μgm:1 U; or 1.39 mole insulin to 1 mole pramlintide;
18 μgm:1 U; or 1.31 mole insulin to 1 mole pramlintide;
19 μgm:1 U; or 1.25 mole insulin to 1 mole pramlintide;
20 μgm:1 U; or 1.18 mole insulin to 1 mole pramlintide;
21 μgm:1 U; or 1.13 mole insulin to 1 mole pramlintide;
22 μgm:1 U; or 1.08 mole insulin to 1 mole pramlintide;
23 μgm:1 U; or 1.03 mole insulin to 1 mole pramlintide;
24 μgm:1 U, or 0.99 mole insulin to 1 mole pramlintide; or
between about 4 or 5 μgm:1 U to about 24 μgm:1 U,
between about 5.5 μgm:1 U to about 16 μgm:1 U,
between about 6 μgm:1 U to about 12 μgm:1 U,
between about 7 μgm:1 U to about 24 μgm:1 U,
between about 7.5 μgm:1 U to about 16 μgm:1 U,
between about 8 μgm:1 U to about 9, 10, 11 or 12 μgm:1 U,
and the liquid pharmaceutical composition or formulation has a pH of between about 3.3 to 4.3, about 3.0 and 5.5, about 3.5 to 4.5, about 3.7 to about 4.35, about 4.0, or a pH of about 3.0, 3.1, 3.2, 3.3, 3.4, 3.5, 3.6, 3.7, 3.8, 3.9, 4.0, 4.05, 4.1, 4.15, 4.2, 4.25, 4.3, 4.35 or 4.4,
or optionally, when the reconstitutable dried pharmaceutical composition or formulation is reconstituted, it has a pH of between about 3.3 to 4.3, about 3.0 and 5.5, about 3.5 to 4.5, about 3.7 to about 4.35, about 4.0, or a pH of about 3.0, 3.1, 3.2, 3.3, 3.4, 3.5, 3.6, 3.7, 3.8, 3.9, 4.0, 4.05, 4.1, 4.15, 4.2, 4.25, 4.3, 4.35 or 4.4;
or optionally, the pH range is more than 3.5 to less than 4.4, or 3.8 to less than 4.4; or pramlintide peptide formulations and final co-formulation pH include about 3.8, 3.85, 3.9, 3.95, 4.0, 4.05, 4.1, 4.15, 4.2, 4.25, 4.3, and 4.35; or the pH range is each range that is selected from the group of ranges where the pH values 3.8, 3.85, 3.9, 3.95, 4.0, 4.05, 4.1, 4.15, 4.2, 4.25, 4.3, and 4.35 are selected as a lower and an upper end of the range, including for example 3.8 to 4.35, 3.85 to 4.35, 3.85 to 4.2, 3.85 to 4.15, 3.8 to 4.2, 3.9 to 4.1, 3.9 to 4.0, and 4.0 to 4.1; or the pH range is about pH 3.9, 4.0 and 4.1, and ranges 3.9 to 4.1, 3.9 to 4.0, and 4.0 to 4.1,
wherein when calculating the ratios, a weight of the pramlintide or pramlintide peptide is based on the weight of pramlintide acetate, and an International Unit (U) of human insulin is based on U of human insulin as formulated using a HUMULIN R™ at pH 7.4;
(b) the liquid or reconstitutable dried pharmaceutical composition or formulation of (a), wherein the pramlintide or pramlintide peptide is or comprises a salt form, and optionally the pramlintide or pramlintide peptide is an acetate salt, or a trifluoroacetate (TFA) salt, or a chloride salt, or a mixture thereof;
(c) the liquid or reconstitutable dried pharmaceutical composition or formulation of (a) or (b), wherein the human insulin or human insulin peptide (HIP) is complexed with a metal ion;
(d) the liquid or reconstitutable dried pharmaceutical composition or formulation of (c), wherein the human insulin or human insulin peptide (HIP) is complexed with a zinc or a Zn⁺²;
(e) the liquid or reconstitutable dried pharmaceutical composition or formulation of (d), wherein the human insulin or human insulin peptide (HIP) is complexed with the zinc in a ratio of molar ratio of at least 6:2;
(f) the liquid or reconstitutable dried pharmaceutical composition or formulation of (d) or (e), wherein the human insulin or human insulin peptide (HIP) is complexed with the zinc and is substantially hexameric;
(g) the liquid or reconstitutable dried pharmaceutical composition or formulation of (f), wherein the human insulin or human insulin peptide (HIP) is complexed with the zinc and the insulin is greater than about 95%, 96%, 97%, 98%, 99% or more hexameric, or is between about 90% and 100% hexameric;
(h) the liquid or reconstitutable dried pharmaceutical composition or formulation of any of (a) to (g), wherein the pramlintide or pramlintide peptide or the insulin or human insulin peptide (HIP) is a recombinant peptide,
and optionally the recombinant peptide is produced in a prokaryote or a eukaryote, and optionally the prokaryote is an E. coli, and optionally the eukaryote is a yeast; and optionally the yeast is a Saccharomyces or a Pichia,
and optionally where the human insulin or HIP are comprised of an A chain and a B chain, the A chain and B chain are separately synthesized or recombinantly produced, and optionally the recombinant A chain and B chain are synthesized in the same cell;
(i) the liquid pharmaceutical composition or formulation of any of (a) to (h), comprising: a liquid vehicle comprising a water, or an aqueous or an organic solvent mixture, or an substantially isotonic aqueous or organic solvent mixture;
(j) the liquid or reconstitutable dried pharmaceutical composition or formulation of any of claims (a) to (i), further comprising a pharmaceutically acceptable excipient;
(k) the liquid or reconstitutable dried pharmaceutical composition or formulation of any of (a) to (i), further comprising a buffer,
and optionally the buffer comprises an acetate, a phosphate, a citrate, a tartrate, or a glutamate buffer, or a mixture or a combination thereof,
and optionally the buffer is between about 0.02 to 0.5% (w/v) of an acetate, phosphate, citrate, tromethamine or glutamate buffer, or the buffer has an acetate concentration of between about 3.4 and 84.7 mM, a phosphate concentration of between about 2.1 and 52.6 mM, a citrate concentration of between about 1.1 and 26.4 mM, or a glutamate concentration of between about 1.4 and 34.2 mM;
(l) the liquid or reconstitutable dried pharmaceutical composition or formulation of (k), wherein the buffer is an acetate buffer, and optionally the acetate is formulated at between about 15 to 20 mM, 17 to 25 mM, 25 to 65 mM, or about 25 to 80 mM or is formulated at about 15 mM, 16 mM, 17 mM, 20 mM, 25 mM, 30 mM, 40 mM, 50 mM, 60 mM, 70 mM, 80 mM, 90 mM, 100 mM, 110 mM or 120 mM,
and optionally the buffer does not or substantially does not chelate a zinc, and optionally for the reconstitutable dried pharmaceutical composition or formulation, the buffer is a non-volatile buffer;
(m) the liquid or reconstitutable dried pharmaceutical composition or formulation of any of (k) to (l), wherein the buffer is present at a concentration providing a buffer capacity equivalent to the buffer capacity of sodium acetate buffer formulated at between about 15 to 20 mM, 17 to 25 mM, 25 to 65 mM, 25 to 80 mM, or at about 15 mM, 16 mM, 17 mM, 20 mM, 25 mM, 30 mM, 40 mM, 50 mM, 60 mM, 70 mM, 80 mM, 90 mM, 100 mM, 110 mM or 120 mM,
(n) the liquid or reconstitutable dried pharmaceutical composition or formulation of any of (a) to (m), further comprising an isotonicity agent or a bulking agent, wherein optionally the isotonicity agent or bulking agent is or comprises a sodium chloride, a carbohydrate, a polyol, or a polyhydric alcohol or a combination or mixture thereof;
(o) the liquid or reconstitutable dried pharmaceutical composition or formulation of (n), wherein the isotonicity agent carbohydrate or polyhydric alcohol or amino acid is formulated as a substantially isotonic formulation, optionally at about 1.0 to 10% (w/v) of the carbohydrate or the polyhydric alcohol or amino acid;
(p) the liquid or reconstitutable dried pharmaceutical composition or formulation of any of (n) to (o), wherein the polyhydric alcohol comprises a mannitol (D-mannitol), a sorbitol, an inositol, a glycerol, a xylitol, an ethylene glycol, a propylene/ethylene glycol copolymer, a PEG 8000, a PEG 400, a PEG 4000, a PEG 200, a PEG 1450 or a PEG 3350, or a combination thereof;
(q) the liquid or reconstitutable dried pharmaceutical composition or formulation of any of (n) to (p), wherein the carbohydrate comprises a mannitol, a mannose, a ribose, a trehalose, a maltose, a glycerol, a inositol, a lactose, a sucrose, a fructose, a galactose, or an arabinose, or a mixture or a combination thereof;
(r) the liquid or reconstitutable dried pharmaceutical composition or formulation of any of (a) to (q), wherein further comprising a glycerol, a glycerin, a mannitol, glycine or a mixture or a combination thereof,
and optionally the glycerol, when present, is between about 12 to 20 mg/ml, or about 16 mg/ml, and the mannitol, when present, is between about 3% to 6%, or about 4.3% (w/v);
(s) the liquid or reconstitutable dried pharmaceutical composition or formulation of any of (a) to (r), further comprising an isotonicity agent comprising a mixture of a glycerol and a mannitol;
(t) the liquid or reconstitutable dried pharmaceutical composition or formulation of any of (a) to (s), further comprising a preservative, wherein optionally the buffer is a meta-cresol (or m-cresol, m-methylphenol, or m-methylphenylol) or a phenol,
and optionally the m-cresol is formulated at between about 2 and 4 mg/mL, or at about 3 mg/mL, 2.25 mg/mL, 2.5 mg/mL or 2.0 mg/mL, wherein optionally the m-cresol is formulated at one-half of between about 2 and 4 mg/mL, or at about 3 mg/mL, 2.25 mg/mL, 2.5 mg/mL or 2.0 mg/mL, due to dilution;
(u) the liquid or reconstitutable dried pharmaceutical composition or formulation of any of (a) to (t), further comprising a metal ion, and optionally the metal ion is or comprises: a salt of a metal ion, a zinc or a Zn⁺²,
wherein optionally the metal salt is a zinc chloride, a zinc acetate, a zinc oxide and optionally the zinc chloride is formulated at about 7 micrograms/mL (mgm/mL), and optionally the Zn⁺² is formulated at an amount equivalent to a zinc in a zinc chloride at about 7 mcg/mL, wherein optionally the zinc is formulated at about 0.015 mg/100 units;
(v) the liquid or reconstitutable dried pharmaceutical composition or formulation of any of (a) to (u), further comprising a surfactant,
and optionally the surfactant comprises a polyoxyethylene (20) sorbitan monolaurate, a polyoxyethylene (20) sorbitan monooleate, a 3-[(3-cholamidopropyl) dimethylammonio]1-propanol sulfonate, a polyoxyethylene (23) lauryl ether, a poloxamer or a non-ionic surfactant or a mixture or combination thereof;
(w) the liquid or reconstitutable dried pharmaceutical composition or formulation of any of (a) to (v), wherein the dried pharmaceutical composition or formulation is prepared by spray drying, rotary evaporation, freeze-drying or lyophilisation;
(x) the liquid or reconstitutable dried pharmaceutical composition or formulation of any of (a) to (w), comprising or formulated as: an aqueous solution, an injectable solution, an aqueous or an organic solvent mixture, a suspension, a lozenge, a capsule, a gel, a geltab, a nanosuspension, a nanoparticle, a microgel and/or a spray or an aerosol;
(y) the liquid or reconstitutable dried pharmaceutical composition or formulation of any of (a) to (x), comprising or packaged in: a continuous subcutaneous insulin infusion therapy device; an insulin pump device; an ampoule; a vial; a cartridge; a syringe, cartridge or disposable pen or jet injector; a needleless injector or a needle free injector; a prefilled pen or syringe or cartridge, or a disposable syringe or pen or jet injector; an AUTOPEN™; a two chambered syringe, cartridge or disposable pen or jet injector; a multi-chambered syringe, cartridge or disposable pen or jet injector;
(z) the liquid or reconstitutable dried pharmaceutical composition or formulation of any of (a) to (y), further comprising instructions for using the liquid pharmaceutical composition or formulation to treat a patient, wherein optionally the patient is being treated for, and the instructions are for use of the liquid or reconstitutable dried pharmaceutical composition or formulation for treating:
a diabetes mellitus (diabetes), wherein optionally the diabetes mellitus is Type 1 diabetes or Type 2 diabetes, or a prediabetic condition (prediabetes),
a dementia or Alzheimer's disease,
an abnormality of blood glucose control, or inability to control blood glucose
an elevation of fasting glucose or Impaired Fasting Glucose (IFG),
an abnormality of tolerance to a glucose load or Impaired Glucose Tolerance (IGT),
a hyperglycemia induced by an illness, a trauma, a medication administration or a form of metabolic, psychological or physical stress, or a hyperglycemia induced by steroids (steroid-induced diabetes),
a latent autoimmune diabetes in adults (LADA),
a postprandial or reactive Hypoglycemia or an insulin resistance,
a PolyCystic Ovary Syndrome (PCOS),
a ketoacidosis,
a gestational diabetes,
a hyperkalemia,
a cancer or cachexia,
a beta blocker overdose, or
a jaundice,
and optionally the patient is being treated with a basal insulin, or the insulin is administered to maintain a basal insulin level,
and optionally the patient is treated with an oral or injectable anti-diabetic medicine, or one or more other medications, or the patients can be those naïve to insulin other anti-diabetes medicines, and whether naïve or not, the formulations of the invention can be the patients only anti-diabetes medication;
(aa) the liquid or reconstitutable dried pharmaceutical composition or formulation of any of (a) to (z), wherein the liquid insulin concentration is at about 100 Units/mL, 200 Units/mL, 300 Units/mL, 400 Units/mL, 500 Units/mL or 600 Units/mL, or is between about 100 Units/mL to about 600 Units/mL,
or the reconstitutable dried pharmaceutical composition or formulation is formulated such that upon reconstitution the liquid insulin concentration will be at about 100 Units/mL, 200 Units/mL, 300 Units/mL, 400 Units/mL, 500 Units/mL or 600 Units/mL, or will be between about 100 Units/mL to about 600 Units/mL,
and optionally the reconstitutable dried pharmaceutical composition or formulation is reconstituted by a health practitioner or by a pharmacist, or is reconstituted by a patient;
(bb) the liquid or reconstitutable dried pharmaceutical composition or formulation of any of (a) to (aa), wherein the liquid formulation or the reconstituted pharmaceutical composition or formulation is usable by a patient for 1 day to 1 month, or for 1 day to 7 days, or for 1 day to 3 days, or for 1 day, 3 days, 1 week, 2 weeks or 1 month; or
(cc) the liquid or reconstitutable dried pharmaceutical composition or formulation of any of (a) to (bb), comprising or consisting of:
a formulation as set forth in FIGS. 8 to 11, 34A, 34B, 34C, 34D or 34E, or FIG. 35; or
an insulin 100 U/mL, pramlintide 600 microgram/mL, in 30 mM acetate buffer pH 4, 0.225% metra-cresol, 4.3% mannitol; or
an insulin 100 U/mL, pramlintide 900 microgram/mL, in 30 mM acetate buffer pH 4, 0.225% metra-cresol, 4.3% mannitol; or
a lyophilized insulin 1000 U powder with bulking agent+10 mL of Pramlintide solution where Pramlintide 600 microgram/mL in 30 mM acetate buffer pH 4, 0.225% metra-cresol, 4.3% mannitol; or
a lyophilized insulin 1000 U powder with bulking agent+10 mL of Pramlintide solution where Pramlintide 900 microgram/mL in 30 mM acetate buffer pH 4, 0.225% metra-cresol, 4.3% mannitol.

2: A device or product of manufacture, a subcutaneous insulin infusion therapy device; a continuous subcutaneous insulin infusion therapy device; an insulin pump device; an ampoule; a vial; a cartridge; a syringe, cartridge or disposable pen or jet injector; a needleless injector or a needle free injector; a prefilled syringe or pen or cartridge, or a disposable pen or syringe or jet injector; a two chambered syringe, cartridge or disposable pen or jet injector; a multi-chambered syringe, cartridge or disposable pen or jet injector; or a kit, comprising:
(a) the liquid or reconstitutable dried pharmaceutical composition or formulation of claim 1,
wherein optionally the human insulin or human insulin peptide (HIP) and the pramlintide or pramlintide peptide are co-formulated together as a liquid, or
optionally the human insulin or human insulin peptide (HIP) and the pramlintide or pramlintide peptide are co-formulated together as a reconstitutable dried pharmaceutical composition or formulation, or
optionally the human insulin or human insulin peptide (HIP) and the pramlintide or pramlintide peptide are separately formulated and are stored or self-contained separately before mixing to comprise a liquid pharmaceutical composition or formulation of claim 1;
(b) the device or product of manufacture of (a) or (b), further comprising an actuator, a valve, a shunt, a directional channel or equivalent thereof, or an apparatus capable of delivering or administrating to a patient or an individual an therapeutically effective dosage equivalent to a dosage of the liquid or reconstitutable dried pharmaceutical composition or formulation of claim 1;
(c) the device or product of manufacture of (a) or (b), wherein the human insulin or human insulin peptide (HIP) and the pramlintide or pramlintide peptide are separately formulated and are stored separately,
and optionally the human insulin or human insulin peptide (HIP) and the pramlintide or pramlintide peptide are stored separately in separate, different or multicompartment ampoules, capsules, compartments, vials, sections, cartridges, or equivalents thereof, or in separate sections or areas of a multi-compartment cartridge, ampoule, vial or capsule or equivalents thereof,
and optionally the device or product of manufacture can deliver, or is configured to deliver, the pramlintide or pramlintide peptide and the human insulin or human insulin peptide (HIP) at a ratio as set forth in claim 1,
and optionally the actuator, a valve, a shunt, a directional channel or equivalent thereof are manufactured or configured to deliver, the pramlintide or pramlintide peptide and the human insulin or human insulin peptide (HIP) at a ratio as set forth in claim 1,
and optionally the actuator, a valve, a shunt, a directional channel or equivalent thereof are operably linked to a computer system, a non-transitory memory medium, a computer-readable storage medium or a computer program storage device, or an equivalent thereof, to deliver the pramlintide or pramlintide peptide and the human insulin or human insulin peptide (HIP) at a ratio as set forth in claim 1;
and optionally computer system, non-transitory memory medium, computer-readable storage medium or computer program storage device, or equivalent thereof are operably linked to or are built into or are part of the device or product of manufacture;
(d) the device or product of manufacture of any of (a) to (c), wherein:
the pramlintide or pramlintide peptide is in a liquid formulation and the human insulin or human insulin peptide (HIP) is formulated in a dried formulation, such that when the pramlintide or pramlintide peptide and insulin or insulin peptide are mixed the mixture comprises a liquid formulation as set forth in claim 1,
the human insulin or human insulin peptide (HIP) is in a liquid formulation and the pramlintide or pramlintide peptide is formulated in a dried formulation, such that when the pramlintide or pramlintide peptide and human insulin or human insulin peptide (HIP) are mixed the mixture comprises a liquid formulation as set forth in claim 1, or
the insulin or insulin peptide and the pramlintide or pramlintide peptide are both formulated in a dried formulation, such that when the pramlintide or pramlintide peptide and human insulin or human insulin peptide (HIP) are mixed the mixture comprises a liquid formulation as set forth in claim 1,
and optionally the liquid for reconstituting the dried formulation or formulations are contained in or stored in or within the device or product of manufacture, or, the device or product of manufacture is configured or manufactured to receive input of a liquid to reconstitute the dried formulation;
(e) the device or product of manufacture of any of (a) to (d), wherein the human insulin or human insulin peptide (HIP) in formulated as a liquid formulation and the insulin is a HUMULIN R™ or a NOVOLIN R™ formulation and the HIP is a HUMALOG™, NOVALOG™, or APIDRA™ formulation;
(f) the device or product of manufacture of any of (a) to (e), wherein the pramlintide or pramlintide peptide is formulated as a liquid formulation having a buffer capacity equivalent to that of at least 30 mM sodium acetate, or that of at least greater than 30 mM acetate, or at least greater than 30 mM to 80 mM sodium acetate;
(g) the device or product of manufacture of any of (a) to (f), wherein the pramlintide or pramlintide peptide is formulated in a liquid SYMLIN™ formulation;
(h) the device or product of manufacture of any of (a) to (g), wherein the pramlintide or pramlintide peptide is formulated in a liquid SYMLIN™ formulation that optionally further comprises a buffer capacity equivalent to that of at least 30 mM sodium acetate, or that of at least greater than 30 mM acetate, or at least greater than 30 mM to 80 mM sodium acetate; or
optionally the buffer comprises an acetate, a phosphate, a citrate, a tartrate, or a glutamate buffer, or a mixture or a combination thereof,
and optionally the buffer is between about 0.02 to 0.5% (w/v) of an acetate, phosphate, citrate or glutamate buffer, or the buffer has an acetate concentration of between about 3.4 and 84.7 mM, a phosphate concentration of between about 2.1 and 52.6 mM, a citrate concentration of between about 1.1 and 26.4 mM, or a glutamate concentration of between about 1.4 and 34.2 mM;
and optionally the buffer is an acetate buffer, and optionally the acetate is formulated at between about 15 to 20 mM, 17 to 25 mM, 25 to 65 mM, or about 25 to 80 mM or is formulated at about 15 mM, 16 mM, 17 mM, 20 mM, 25 mM, 30 mM, 40 mM, 50 mM, 60 mM, 70 mM, 80 mM, 90 mM, 100 mM, 110 mM or 120 mM, and optionally the buffer is present at a concentration providing a buffer capacity equivalent to the buffer capacity of sodium acetate buffer formulated at between about 15 to 20 mM, 17 to 25 mM, 25 to 65 mM, 25 to 80 mM, or at about 15 mM, 16 mM, 17 mM, 20 mM, 25 mM, 30 mM, 40 mM, 50 mM, 60 mM, 70 mM, 80 mM, 90 mM, 100 mM, 110 mM or 120 mM,
and optionally the buffer does not or substantially does not chelate a zinc, and optionally for the reconstitutable dried pharmaceutical composition or formulation, the buffer is a non-volatile buffer;
(i) the device of product wherein the liquid or reconstitutable formulation of claim 1 is delivered as a bolus prior to a meal to reduce the meal-associated blood glucose rise, and optionally the human insulin or HIP or the liquid or reconstitutable formulation of claim 1 is delivered in sufficient amounts at sufficient time intervals to maintain a basal level of insulin activity, and optionally wherein the basal level is different during waking versus sleeping.

3: A method for treating or ameliorating:
a diabetes mellitus (diabetes), wherein optionally the diabetes mellitus is Type 1 diabetes or Type 2 diabetes, or a prediabetic condition (prediabetes);
a dementia or Alzheimer's disease;
an abnormality of blood glucose control, or inability to control blood glucose,
an elevation of fasting glucose or Impaired Fasting Glucose (IFG),
an abnormality of tolerance to a glucose load or Impaired Glucose Tolerance (IGT),
a hyperglycemia induced by an illness, a trauma, a medication administration or a form of metabolic, psychological or physical stress, or a hyperglycemia induced by steroids (steroid-induced diabetes),
a latent autoimmune diabetes in adults (LADA),
a postprandial or reactive Hypoglycemia or an insulin resistance,
a PolyCystic Ovary Syndrome (PCOS),
a ketoacidosis,
a gestational diabetes,
a hyperkalemia,
a cancer or cachexia,
a beta blocker overdose, or
a jaundice,
in an individual or a patient in need of such treatment comprising:
administering a therapeutically effective amount of the liquid, reconstitutable dried pharmaceutical composition or formulation of claim 1 to the individual or patient in need of such treatment, or
delivering to the individual or patient in need of such treatment a pramlintide or pramlintide peptide and human insulin or human insulin peptide (HIP) formulation at a ratio as set forth in claim 1 using a device, a product of manufacture, an insulin pump; a subcutaneous insulin infusion therapy device, a continuous subcutaneous insulin infusion therapy device; an insulin pump device; an ampoule; a vial; a cartridge; a syringe, cartridge or disposable pen or jet injector; a needleless injector or a needle free injector; a prefilled syringe, cartridge or disposable pen or jet injector; a two chambered syringe, cartridge or disposable pen or jet injector; a multi-chambered syringe, cartridge or disposable pen or jet injector; as set forth in claim 2,
and optionally a reconstitutable dried pharmaceutical composition or formulation is reconstituted by a health practitioner or by a pharmacist, or is reconstituted by a patient.
and optionally the diabetes is a Type 1 or a Type 2 diabetes; and optionally the patient is taking an additional basal insulin supplement,
and optionally the patient is treated with one or more oral or injectable anti-diabetic medicine, or one or more other medications,
and optionally an insulin or insulin bolus administered to an individual can be reduced by approximately 21% at a P/I ratio of about 9 μg/U (or 9 μg pramlintide or pramlintide peptide to 1 U human insulin or human insulin peptide (HIP)) to account for the in vivo effects of pramlintide and to avoid a postprandial hypoglycaemia.

4: A method for treating or ameliorating:
a diabetes mellitus (diabetes), wherein optionally the diabetes mellitus is Type 1 diabetes or Type 2 diabetes, or a prediabetic condition (prediabetes);
a dementia or Alzheimer's disease;
an abnormality of blood glucose control, or inability to control blood glucose,
an elevation of fasting glucose or Impaired Fasting Glucose (IFG),
an abnormality of tolerance to a glucose load or Impaired Glucose Tolerance (IGT),
a hyperglycemia induced by an illness, a trauma, a medication administration or a form of metabolic, psychological or physical stress, or a hyperglycemia induced by steroids (steroid-induced diabetes),
a latent autoimmune diabetes in adults (LADA),
a postprandial or reactive Hypoglycemia or an insulin resistance,
a PolyCystic Ovary Syndrome (PCOS),
a ketoacidosis,
a gestational diabetes,
a hyperkalemia,
a cancer or cachexia,
a beta blocker overdose, or
a jaundice,
in an individual or a patient in need of such treatment comprising administering a therapeutically effective amount of:
(a) (i) a pramlintide or pramlintide peptide, or a physiologically acceptable salt thereof; and
(ii) a human insulin or a human insulin peptide (HIP) or an analog thereof, or a physiologically acceptable salt thereof,
wherein the ratio of the pramlintide or pramlintide peptide to the human insulin or human insulin peptide (HIP) is administered to the individual or patient is:
4 μgm:1 U; or 5.92 mole insulin to 1 mole pramlintide;
4.5 μgm:1 U; or 5.26 mole insulin to 1 mole pramlintide;
5 μgm:1 U; or 4.74 mole insulin to 1 mole pramlintide;
5.5 μgm:1 U; or 4.31 mole insulin to 1 mole pramlintide;
6 μgm or 1.52 nmoles pramlintide:1 U (international unit) or 6.0 nmoles human insulin or equivalent nmoles of human insulin peptide providing 1 U of insulin activity, or 0.25 mole pramlintide to 1 mole human insulin (6 μgm:1 U) or moles of human insulin peptide providing the same unit of activity as 0.25 mole human insulin, or 3.98 mole human insulin or equivalent moles of human insulin peptide to 1 mole pramlintide;
6.5 μgm:1 U; or 3.63 mole insulin to 1 mole pramlintide;
7 μgm:1 U; or 3.38 mole insulin to 1 mole pramlintide;
8 μgm:1 U; or 2.96 mole insulin to 1 mole pramlintide;
8.5 μgm:1 U; or 2.79 mole insulin to 1 mole pramlintide;
9 μgm:1 U; or 2.63 mole insulin to 1 mole pramlintide;
9.5 μgm:1 U; or 2.49 mole insulin to 1 mole pramlintide;
10 μgm:1 U; or 2.37 mole insulin to 1 mole pramlintide;
11 μgm:1 U; or 2.15 mole insulin to 1 mole pramlintide;
12 μgm:1 U; or 1.97 mole insulin to 1 mole pramlintide;
13 μgm:1 U; or 1.82 mole insulin to 1 mole pramlintide;
14 μgm:1 U; or 1.69 mole insulin to 1 mole pramlintide;
15 μgm:1 U; or 1.58 mole insulin to 1 mole pramlintide;
16 μgm:1 U; or 1.48 mole insulin to 1 mole pramlintide;
17 μgm:1 U; or 1.39 mole insulin to 1 mole pramlintide;
18 μgm:1 U; or 1.31 mole insulin to 1 mole pramlintide;
19 μgm:1 U; or 1.25 mole insulin to 1 mole pramlintide;
20 μgm:1 U; or 1.18 mole insulin to 1 mole pramlintide;
21 μgm:1 U; or 1.13 mole insulin to 1 mole pramlintide;
22 μgm:1 U; or 1.08 mole insulin to 1 mole pramlintide;
23 μgm:1 U; or 1.03 mole insulin to 1 mole pramlintide;
24 μgm:1 U, or 0.99 mole insulin to 1 mole pramlintide; or
between about 4 μgm:1 U to about 24 μgm:1 U,
between about 5 μgm:1 U to about 24 μgm:1 U,
between about 5.5 μgm:1 U to about 16 μgm:1 U,
between about 6 μgm:1 U to about 12 μgm:1 U,
between about 7 μgm:1 U to about 24 μgm:1 U,
between about 7.5 μgm:1 U to about 16 μgm:1 U, or
between about 8 μgm:1 U to about 9, 10, 11 or 12 μgm:1 U,
wherein when calculating the ratios, a weight of the pramlintide or pramlintide peptide is based on the weight of pramlintide acetate, and an International Unit (U) of human insulin is based on U of human insulin as formulated using a HUMULIN R™ at pH 7.4;
(b) the method of (a), wherein the pramlintide or pramlintide peptide is or comprises a SYMLIN™;
(c) the method of (a) or (b), wherein the human insulin or human insulin peptide (HIP) is or comprises a recombinant peptide, a NOVOLIN R™, or a HUMULIN® R U-100™;
(d) the method of any of (a) to (c), wherein the human insulin or human insulin peptide (HIP) is admixed with the pramlintide or pramlintide peptide prior to administration, or simultaneously at delivery (administration) to the individual or patient, or, the admixing step is simultaneous, or concerted and sequential with administration;
(e) the method of any of (a) to (c), wherein a reconstitutable dried pharmaceutical composition or formulation is reconstituted by a health practitioner or by a pharmacist, or is reconstituted by a patient, or
(f) the method of any of (a) to (e), wherein the amount of an insulin or insulin bolus administered to an individual can be reduced by approximately 21% at a P/I ratio of about 9 μg/U (or 9 μg pramlintide or pramlintide peptide to 1 U human insulin or human insulin peptide (HIP)) to account for the in vivo effects of pramlintide and to avoid a postprandial hypoglycaemia.

5: The method of claim 4, wherein before admixing the human insulin or human insulin peptide (HIP) and the pramlintide or pramlintide peptide are stored separately, or stored separately in separate or different: insulin pumps; devices, subcutaneous insulin infusion therapy devices, continuous subcutaneous insulin infusion therapy devices, infusion therapy devices, reservoirs, ampoules, vials, syringes, cartridges, disposable pen or jet injectors, needleless injectors, needle free injectors, prefilled pens or syringes or cartridges, cartridge or disposable pen or jet injectors; or are stored in separate reservoirs or chambers in a subcutaneous insulin infusion therapy device, a continuous subcutaneous insulin infusion therapy device, a two chambered or multi-chambered pump, syringe, cartridge or pen or jet injector,
wherein optionally the separate or different insulin pumps; devices, subcutaneous insulin infusion therapy devices, continuous subcutaneous insulin infusion therapy devices, infusion therapy devices, reservoirs, ampoules, vials, cartridges, syringes, cartridges, disposable pen or jet injectors, prefilled pens or syringes or cartridges, or disposable pen or jet injectors, or needleless injectors or needle free injectors, comprise separate or at least two or more pramlintide or pramlintide peptides and insulin formulations and deliver a final pramlintide or pramlintide peptide and human insulin or human insulin peptide (HIP) formulation dosage, or a pramlintide and human insulin or human insulin peptide (HIP) effective dosage, at a ratio as set forth in claim 1;
an optionally the pramlintide or pramlintide peptide and human insulin or human insulin peptide (HIP) are delivered or administered a pre-meal bolus, and optionally where the human insulin or HIP or the pramlintide or pramlintide peptide and human insulin or human insulin peptide (HIP) are delivered or administered to provide a basal level of insulin activity,
wherein the insulin and the pramlintide or pramlintide peptide are delivered to the patient or individual using a device or product of manufacture.

6. (canceled)

7: A liquid or a reconstitutable dried pharmaceutical composition or formulation for use in treating or ameliorating:
a diabetes mellitus (diabetes), wherein optionally the diabetes mellitus is Type 1 diabetes or Type 2 diabetes, or a prediabetic condition (prediabetes);
a dementia or Alzheimer's disease;
an abnormality of blood glucose control, or inability to control blood glucose,
an elevation of fasting glucose or Impaired Fasting Glucose (IFG),
an abnormality of tolerance to a glucose load or Impaired Glucose Tolerance (IGT),
a hyperglycemia induced by an illness, a trauma, a medication administration or a form of metabolic, psychological or physical stress, or a hyperglycemia induced by steroids (steroid-induced diabetes),
a latent autoimmune diabetes in adults (LADA),
a postprandial or reactive Hypoglycemia or an insulin resistance,
a PolyCystic Ovary Syndrome (PCOS),
a ketoacidosis,
a gestational diabetes,
a hyperkalemia,
a cancer or cachexia,
a beta blocker overdose, or
a jaundice,
comprising the liquid or reconstitutable dried pharmaceutical composition or formulation of claim 1.

8-11. (canceled)

12: A therapeutic combination of drugs comprising or consisting of a combination of at least two compounds: wherein the at least two compounds comprise or consist of:
(a) (i) a pramlintide or pramlintide peptide or a physiologically acceptable salt thereof; and
(ii) a human insulin, or a Human Insulin Peptide (HIP), or an analog thereof, or a physiologically acceptable salt thereof,
and optionally the human insulin peptide (HIP) or analog thereof is or comprises: an aspart, a NOVOLOG™ or a NOVORAPID™ (Novo Nordisk, Bagsværd, Denmark); a glulisine or an APIDRA™ (Sanofi S.A., Paris, France); a lispro, an insulin lispro protamine or a HUMALOG™ (Eli Lilly and Company, Indianapolis, Ind.); a HUMULIN R™, a HUMULIN N™, a HUMULIN 70/30™ or a HUMULIN 70/30™ (Eli Lilly and Company, Indianapolis, Ind.);
or a regular (wild type) isolated or a recombinant human insulin, or a fast-acting human insulin analog or variant thereof,
and optionally the pramlintide peptide comprises or consists of a C-terminal amide form of KCNTATCATQRLANFLVHSSNNFGPILPPTNVGSNTY (SEQ ID NO:1);
wherein the ratio of the pramlintide or pramlintide peptide to the insulin or insulin peptide administered to an individual or patient is a ratio as set forth in claim 1;
(b) the therapeutic combination of drugs of (a), wherein the pramlintide or pramlintide peptide is or comprises pramlintide acetate; or
(c) the therapeutic combination of drugs of (a) or (b), wherein the insulin or the pramlintide or pramlintide peptide is or comprises a recombinant peptide.

13: A combination for ameliorating, diminishing, treating, blocking or preventing:
a diabetes mellitus (diabetes), wherein optionally the diabetes mellitus is Type 1 diabetes or Type 2 diabetes, or a prediabetic condition (prediabetes);
a dementia or Alzheimer's disease;
an abnormality of blood glucose control, or inability to control blood glucose,
an elevation of fasting glucose or Impaired Fasting Glucose (IFG),
an abnormality of tolerance to a glucose load or Impaired Glucose Tolerance (IGT),
a hyperglycemia induced by an illness, a trauma, a medication administration or a form of metabolic, psychological or physical stress, or a hyperglycemia induced by steroids (steroid-induced diabetes),
a latent autoimmune diabetes in adults (LADA),
a postprandial or reactive Hypoglycemia or an insulin resistance,
a PolyCystic Ovary Syndrome (PCOS),
a ketoacidosis,
a gestational diabetes,
a hyperkalemia,
a cancer or cachexia,
a beta blocker overdose, or
a jaundice,
comprising:
(a) (i) a pramlintide or pramlintide peptide or a physiologically acceptable salt thereof; and
(ii) a human insulin, or a Human Insulin Peptide (HIP), or an analog thereof, or a physiologically acceptable salt thereof,
and optionally the human insulin peptide (HIP) or analog thereof is or comprises: an aspart, a NOVOLOG™ or a NOVORAPID™ (Novo Nordisk, Bagsværd, Denmark); a glulisine or an APIDRA™ (Sanofi S.A., Paris, France); a lispro, an insulin lispro protamine or a HUMALOG™ (Eli Lilly and Company, Indianapolis, Ind.); a HUMULIN R™, a HUMULIN N™, a HUMULIN 70/30™ or a HUMULIN 70/30™ (Eli Lilly and Company, Indianapolis, Ind.),
or a regular (wild type) isolated or a recombinant human insulin, or a fast-acting human insulin analog or variant thereof,
and optionally the pramlintide peptide comprises or consists of a C-terminal amide form of KCNTATCATQRLANFLVHSSNNFGPILPPTNVGSNTY (SEQ ID NO:1);
wherein the ratio of the pramlintide or pramlintide peptide to the insulin or insulin peptide administered to an individual or patient is a ratio as set forth in claim 1;
(b) the combination of (a), wherein the pramlintide or pramlintide peptide is or comprises a pramlintide acetate; or
(c) the combination of (a) or (b), wherein the insulin or the pramlintide or pramlintide peptide is or comprises a recombinant peptide.

14: A computer-implemented method capable of calculating a ratio of the amount of:
(a) a pramlintide or pramlintide peptide or a physiologically acceptable salt thereof; and
(b) a human insulin, or a Human Insulin Peptide (HIP), or an analog thereof, or a physiologically acceptable salt thereof,
to be delivered to a patient or an individual in need thereof, wherein the ratio of the pramlintide or pramlintide peptide to the insulin or insulin peptide administered to the individual or patient is a ratio as set forth in claim 1.

15: A computer-implemented method of processing data, wherein the method calculates a ratio of the amount of:
(a) a pramlintide or pramlintide peptide or a physiologically acceptable salt thereof; and
(b) a human insulin, or a Human Insulin Peptide (HIP), or an analog thereof, or a physiologically acceptable salt thereof,
to be delivered to a patient or individual in need thereof, comprising:
receiving data comprising the insulin level, or basal insulin level, in the patient or individual;
storing the data elements in a memory; and
calculating the ratio of the pramlintide or pramlintide peptide to the insulin or insulin peptide to be administered to the individual or patient, wherein the ratio is as set forth in claim 1,
and optionally the patient or individual in need thereof is under therapeutic or preventative treatment for:
a diabetes mellitus (diabetes), wherein optionally the diabetes mellitus is Type 1 diabetes or Type 2 diabetes, or a prediabetic condition (prediabetes);
a dementia or Alzheimer's disease;
an abnormality of blood glucose control, or inability to control blood glucose,
an elevation of fasting glucose or Impaired Fasting Glucose (IFG),
an abnormality of tolerance to a glucose load or Impaired Glucose Tolerance (IGT),
a hyperglycemia induced by an illness, a trauma, a medication administration or a form of metabolic, psychological or physical stress, or a hyperglycemia induced by steroids (steroid-induced diabetes),
a latent autoimmune diabetes in adults (LADA),
a postprandial or reactive Hypoglycemia or an insulin resistance,
a PolyCystic Ovary Syndrome (PCOS),
a ketoacidosis,
a gestational diabetes,
a hyperkalemia,
a cancer or cachexia,
a beta blocker overdose, or
a jaundice.

16: The computer-implemented method of claim 15, further comprising being operably connected to and communicating to one or separate or different: devices, insulin pumps, subcutaneous insulin infusion therapy devices, continuous subcutaneous insulin infusion therapy devices, infusion therapy devices, reservoirs, ampoules, vials, cartridges, syringes, cartridges, disposable pen or jet injectors, prefilled pens or syringes or cartridges, or disposable pen or jet injectors, or needleless injectors or needle free injectors, wherein the pramlintide or pramlintide peptide and insulin are stored in separate reservoirs or chambers therein, and actuating or causing the insulin to be admixed with the pramlintide or pramlintide peptide prior to administration, or actuating or causing a simultaneously delivery (administration) to the individual or patient, or, actuating or causing an admixing step that is simultaneous, or concerted and sequential with administration.

17: A computer program product for processing data, or a Graphical User Interface (GUI) computer program product, the computer program product comprising the computer-implemented method of processing data of claim 15.

18: A computer system comprising a processor and a data storage device wherein said data storage device has stored thereon a computer program product for processing data, or a Graphical User Interface (GUI) computer program product, of claim 17.

19: A non-transitory memory medium comprising program instructions for running, processing and/or implementing a computer program product for processing data, or a Graphical User Interface (GUI) computer program product, of claim 17.

20: A computer-readable storage medium comprising a set of or a plurality of computer-readable instructions that, when executed by a processor of a computing device, cause the computing device to run, process and/or implement: a computer program product comprising the computer-implemented method of processing data of claim 15.

21: A computer program storage device, embodied on a tangible computer readable medium, comprising: a computer program product of claim 17.

22: A computer or equivalent electronic system, comprising: a memory; and a processor operatively coupled to the memory, the processor adapted to execute program code stored in the memory to: run, process and/or implement: a computer program product of claim 17.

23: A system, comprising: a memory configured to: store values associated with a plurality of data points and/or a plurality of data elements, and a processor adapted to execute program code stored in the memory to: run, process and/or implement: a computer program product of claim 17.

24: A computer-implemented system for providing an application access to an external data source or an external server process via a connection server, and providing the ability to store values associated with the plurality of data points and/or the plurality of data elements, and an application for running, processing and/or implementing: a computer program product of claim 17.

25: A subcutaneous insulin infusion therapy device; a continuous subcutaneous insulin infusion therapy device; an insulin pump device, multi-chambered syringe, cartridge or disposable pen or jet injector, comprising: a computer-implemented method that determines or calculates and activates the delivering or administrating an effective dosage to a patient or individual equivalent
and an actuator or apparatus capable of delivering or administrating an effective dosage to a patient or individual equivalent to a dosage of the liquid, reconstitutable dried or lyophilized pharmaceutical composition or formulation of claim 1,
wherein the computer-implemented system determines or calculates and activates the delivering or administrating an effective dosage to a patient or individual equivalent,
and optionally the insulin pump device, subcutaneous insulin infusion therapy device, continuous subcutaneous insulin infusion therapy device, insulin pump, infusion therapy device, or multi-chambered syringe, cartridge or disposable pen or jet injector, or needleless injector or needle free injector, comprises separate formulations and delivers a pramlintide or pramlintide peptide and insulin formulation at a ratio as set forth in claim 1.


SEQUENCE LISTING (6 sequences)

SEQ ID NO: 1 - protein, 37 aa, artificial sequence
  synthetic polypeptide
KCNTATCATQRLANFLVHSSNNFGPILPPTNVGSNTY

SEQ ID NO: 2 - protein, 21 aa, Homo sapiens
GIVEQCCTSICSLYQLENYCN

SEQ ID NO: 3 - protein, 30 aa, Homo sapiens
FVNQHLCGSHLVEALYLVCGERGFFYTPKT

SEQ ID NO: 4 - protein, 30 aa, artificial sequence
  synthetic polypeptide
FVNQHLCGSHLVEALYLVCGERGFFYTKPT

SEQ ID NO: 5 - protein, 30 aa, artificial sequence
  synthetic polypeptide
FVNQHLCGSHLVEALYLVCGERGFFYTDKT

SEQ ID NO: 6 - protein, 30 aa, artificial sequence
  synthetic polypeptide
FVKQHLCGSHLVEALYLVCGERGFFYTPET